(19) United States
(12) Patent Application Publication
(10) Pub. No.: US 2009/0280493 A1
(43) Pub. Date: Nov. 12, 2009

(54) Methods and Compositions for the Prediction of Response to Trastuzumab Containing Chemotherapy Regimen in Malignant Neoplasia

(75) Inventors: Ralph Markus Wirtz, Koln (DE); Marc Munnes, Erkrath (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Healthcare Diagnostics Inc., Tarrytown, NY (US)

(21) Appl. No.: 12/440,490

(22) PCT Filed: Sep. 4, 2007
(86) PCT No.: PCT/EP07/59283

§ 371 Date: Mar. 9, 2009

(30) Foreign Application Priority Data
Sep. 8, 2006 (EP) 06018836.4

(51) Int. Cl. C12Q 1/68

(52) U.S. Cl. 435/6

(57) ABSTRACT

The invention relates to methods and compositions for the prediction, diagnosis, prognosis, prevention and treatment of neoplastic disease. Neoplastic disease is often caused by chromosomal rearrangements which lead to over- or underexpression of the rearranged genes. The invention discloses genes which are overexpressed in neoplastic tissue and are useful as diagnostic markers and targets for treatment. Methods are disclosed for predicting, diagnosing and prognosing as well as preventing and treating neoplastic disease.

### TECHNICAL FIELD OF THE INVENTION

The invention relates to methods and compositions for the prediction, diagnosis, prognosis, prevention and treatment of neoplastic disease. Neoplastic disease is often caused by chromosomal rearrangements which lead to over- or underexpression of the rearranged genes. The invention discloses genes which are overexpressed in neoplastic tissue and are useful as diagnostic markers and targets for treatment. Methods are disclosed for predicting, diagnosing and prognosing as well as preventing and treating neoplastic disease.

### BACKGROUND OF THE INVENTION

Chromosomal aberrations (amplifications, deletions, inversions, insertions, translocations and/or viral integrations) are of importance for the development of cancer and neoplastic lesions, as they account for deregulations of the respective regions. Amplifications of genomic regions have been described, in which genes of importance for growth characteristics, differentiation, invasiveness or resistance to therapeutic intervention are located. One of those regions with chromosomal aberrations is the region carrying the HER-2/NEU gene which is amplified in breast cancer patients. In approximately 25% of breast cancer patients the HER-2/NEU gene is overexpressed due to gene amplification. HER-2/NEU overexpression correlates with a poor prognosis (relapse, overall survival, sensitivity to therapeutics). The importance of HER-2/NEU for the prognosis of the disease progression has been described [Gusterson et al., 1992, (1)]. Gene specific antibodies raised against HER-2/NEU (Herceptin™) have been generated to treat the respective cancer patients. However, only about 50% of the patients benefit from the antibody treatment with Herceptin™, which is most often combined with chemotherapeutic regimen.

The discrepancy of HER-2/NEU positive tumors (overexpressing HER-2/NEU to similar extent) with regard to responsiveness to therapeutic intervention suggest, that there might be additional factors or genes being involved in growth and apoptotic characteristics of the respective tumor tissues. There seems to be no monocausal relationship between overexpression of the growth factor receptor HER-2/NEU and therapy outcome.

Meanwhile trastuzumab is also approved in early-stage HER-2/NEU-positive breast cancer in Europe and the US. Cardiotoxicity and high cost demand careful selection of patients who may have a benefit. Thusfore an efficacy test of trastuzumab containing therapy is needed.

Measurement of commonly used tumor markers such as estrogen receptor, progesterone receptor, p53 and Ki-67 do provide only very limited information on clinical outcome of specific therapeutic decisions. Therefore there is a great need for a more detailed diagnostic and prognostic classification of tumors to enable improved therapy decisions and prediction of survival of the patients. HER-2/NEU and other markers for neoplastic disease are commonly assayed with diagnostic methods such as immunohistochemistry (IHC) (e.g. HercepTest™ from DAKO Inc.) and Fluorescence-In-Situ-Hybridization (FISH) (e.g. quantitative measurement of the HER-2/NEU and Topoisomerase II alpha with a fluorescence-in-situ-Hybridization kit from VYSIS). Additionally HER-2/NEU can be assayed by detecting HER-2/NEU fragments in serum with an ELISA test (BAYER Corp.) or a with a quantitative PCR kit which compares the amount of HER-2/NEU gene with the amount of a non-amplified control gene in order to detect HER-2/NEU gene amplifications (ROCHE). These methods, however, exhibit multiple disadvantages with regard to sensitivity, specificity, technical and personnel efforts, costs, time consumption, inter-lab reproducibility. These methods are also restricted with regard to measurement of multiple parameters within one patient sample (“multiplexing”). Usually only about 3 to 4 parameters (e.g. genes or gene products) can be detected per tissue slide. Therefore, there is a need to develop a fast and simple test to measure simultaneously multiple parameters in one sample. The present invention addresses the need for additional markers by providing genes, which expression is deregulated in tumors and correlates with clinical outcome. One focus is the deregulation of genes present in specific chromosomal regions and their interaction in disease development and drug responsiveness. Most importantly the detection of genomic alterations at the 17q12, 8q24 and 11q13 genome regions by FISH technology do not address the RNA expression of defined genes within this region and it is generally assumed, that HER-2/NEU, Topoisomerase II alpha, c-myc and CCND1 are the critical genes within this region and that the amplification of the region is equivalent to the presence or overexpression of these genes in breast cancer. However, there are reports of bad correlation of the amplification status and protein expression for some of these markers. Still it is not clear whether the alteration of particular these genes is relevant for prognosis and therapy response. Moreover most studies in this regard focussed on the analysis of surgical resecatates before treatment in the neoadjuvant or adjuvant situation and correlated the results with tumor recurrence at distinct sites or survival due to non-analyzed metastatic lesions.

Apparently, there is a great need for a more detailed diagnostic and prognostic classification of tumors to enable improved therapy decisions and prediction of survival of the patients. The present invention addresses the need for additional markers by providing genes, which expression is deregulated in tumors and correlates with clinical outcome. One focus is the deregulation of genes present in specific chromosomal regions and their interaction in disease development and drug responsiveness.

The present invention addresses these open issues by analyzing pretreatment biopsies with clinical and pathological response of the very same tumor. Moreover, the present invention addresses the need for a fast and simple high-resolution method for determining altered genes associated with cancer status on DNA and RNA level, that is able to detect multiple genes within the 17q12,8q24 and 11q13 regions simultaneously. In addition, it is part of the invention to detect genomic alterations of candidate genes on DNA and RNA level from the very same extract of tiny amounts of tissue, which gives a new diagnostic information on gene content (DNA amount) and correlating gene expression (RNA amount). These assays performed on routine core needle biopsies displaying largely different tumor cell contents ranging from 1% to 90% tumor cell content with or without tissue dissection in an automated and/or manual fashion.

At the San Antonio Breast Cancer Symposium 2005 researchers from the NSABP Operations and Biostatistical Center presented data regarding trastuzumab sensitivity being dependent on coamplification of c-Myc and HER-2/NEU (Kim et al., SABCS Abstract #46). In an effort to identify amplified genes in breast cancer that correlate with poor prognosis in patients treated with standard adjuvant chemotherapy, they have screened for the presence of gene amplification at 27 gene loci (amplicons that are associated with increased mRNA expression) in 1900 cases of node positive breast cancer enrolled in NSABP trial B-28, using fluorescence in situ hybridization (FISH). In multivariate analysis, 3 amplicons (Her-2/neu, cMYC, HTPAP) were associated with poor prognosis independent of other known prognosticators. While co-amplification of Her-2/neu and HTPAP was rare, a significant number of cases had co-amplification of Her-2/neu and cMYC as detected by FISH technology with worse outcome than when each one was amplified alone. This has prompted them to examine the significance of cMYC amplification in Her-2/neu amplified breast cancer treated with trastuzumab. Their a priori hypothesis was that patients with cMYC amplified tumors would derive less benefit from trastuzumab due to independent signaling through cMYC.

In NSABP B-31, 1736 patients with follow-up were randomized to receive adjuvant chemotherapy of 4 cycles of doxorubicin plus cyclophosphamide followed by 4 cycles of paclitaxel with or without trastuzumab, which was given for total of one year beginning with the first cycle of paclitaxel. cMYC FISH results were available from 1549 cases. cMYC was amplified in 432 cases (30%). They examined Recurrence Free Survival as a primary clinical end point. Numbers of events and hazard ratios (HR) of their analysis for recurrence and death are shown below (C=chemotherapy, C+T=chemotherapy and trastuzumab):

| | cMyc not amplified | cMyc amplified | Interaction |
|---|---|---|---|
| | (N = 1078) | (N = 471) | p-value |
| Recurrences | 82/540 (C) vs. | 51/234 (C) vs. | |
| | 55/538 (C + T) | 13/237 (C + T) | |
| HR for Recurrence | 0.63 | 0.24 | 0.007 |
| Deaths | 28/540 (C) vs. | 23/234 (C) vs. | |
| | 29/538 (C + T) | 8/237 (C + T) | |
| HR for death | 0.99 | 0.36 | 0.037 |

The authors concluded, that patients with co-amplification of cMYC and Her-2/neu had worse outcome when treated with chemotherapy alone, addition of trastuzumab reversed this trend, achieving 4 year recurrence free survival of over 90%. Although these data contradict their a priori hypothesis, they discussed, that they were consistent with pre-clinical models that suggested that the pro-apoptotic function of dysregulated cMYC needs to be counterbalanced by an anti-apoptotic signal by another activated oncogene in order for such cells to develop into cancer. They claimed, that amplified Her-2/neu may provide such anti-apoptotic signaling that is reduced by treatment with trastuzumab, resulting in triggering of apoptosis. The hormone receptor status is not addressed in these analysis.

However, in contrast we have found that this assumption focussing on the interaction of c-Myc and Her-2/neu activities itself is not correct and not sufficient for determining sensitivity of breast tumors towards trastuzumab.

### SUMMARY OF THE INVENTION

The present invention is based on discovery that chromosomal alterations in cancer tissues can lead to changes in the expression of genes that are encoded by the altered chromosomal regions 17q12, 8q24 and 11q13. The altered RNA expression of genes within this regions was found to be predictive for response of tumors to chemotherapy, antibody-treatment and (anti)-hormonal treatment. Moreover these genes were of prognostic value in untreated tumor patient cohorts.

By analyzing fresh and fixed tumor tissues from >1000 patients, we found that amplification of genomic DNA frequently occurred as an overexpression of neighboring genes. We therefore named these genomic regions ARCHEONs (“Amplified Regions of Chromosomal Expression Observed in Neoplasia”). Here we have performed high resolution analysis of the genomic regions harboring the Her-2/neu (Chr 17q12), c-Myc (Chr 8q24) and CCND1 (11q13) oncogenes and did RNA expression analysis in FFPE tissues to identify the clinically relevant genes in this regions in order to be able to identify a subgroup of patients exhibiting a response to treatment with trastuzumab in combination with anthracyclin/taxol based chemotherapy.

Moreover, several of these genes can also be detected in body fluids such as nipple aspirates and blood samples (whole blood, serum, plasma). In particular, determination of serum levels of TG, T3, T4, TSH, PRL and autoantibodies raised against TG and TPO in combination with sHer-2/neu and CRP were useful for prognosis and prediction of therapeutic success.

The 17q12, 8q24 and 11q13 loci were investigated as model systems, harboring the Her-2/neu, c-Myc and CCND1 gene. By establishing a high-resolution assay to detect amplification events in neighboring genes, genes that are commonly co-amplified in breast cancer cell lines and patient samples were identified. Exemplary 48 human genes have been identified that are co-amplified on the 17q12 locus (including MLLT6, PCGF2, PSMB3, PIP5K2B, CCDC49, RPL23, LASP1, FBX047, PLXDC1, CACNB1, RPL19, STAC2, FBXL20, PPARBP, CRKRS, NEUROD2, PPP1R1B, STARD3, TCAP, PNMT, PERLD1, ERBB2, Herstatin, C17orf37, GRB7, ZNFN1A3, ZPBP2, ORMDL3, GSDM1, PSMD3, CSF3, THRAP4, THRA, NR1D1, CASC3, RAPGEFL1, WIRE, CDC6, RARA, AK123052 TOP2A, IGFBP4, AF370421, AF417488, CCR7, SMARCE1, BC032815 and MGC45562) in neoplastic lesions from breast cancer tissue resulting in altered expression of several of these genes. Exemplary XX human genes have been identified that are co-amplified on the 8q24 locus in neoplastic lesions from breast cancer tissue (including ZHX2, ZHX1, DERL1, ATAD2, ANXA13, RNF139, FBX032, MTSS1, TRIB1, NSE2, c-Myc, MLZE, FAM49B, DDEF1, ADCY8, KIAA0143, WISP1, TG, SLA, NDRG1, ST3GAL1) resulting in altered expression of several of these genes. Exemplary 9 human genes have been identified that are co-amplified on the 11q13 locus in neoplastic lesions from breast cancer tissue (including MYEOV, CCND1, ORAOV1, FGF19, FGF4, FGF3, TMEM16A, FADD and PPFIA1) resulting in altered expression of several of these genes.

The 43 genes on 17q12 are differentially expressed in breast cancer states, relative to their expression in normal, or non-breast cancer states. By gene array technologies and immunological methods their co-overexpression in tumor samples was demonstrated. Surprisingly, by clustering tissue samples with Her-2/neu positive Tumor samples, it was found that the expression pattern of this larger genomic region (consisting of 43 genes) is very similar to control brain tissue. Her-2/neu negative breast tumor tissue did not show a similar expression pattern. Indeed, some of the genes within these cluster are important for neural development (Her-2/neu, THRA) in mouse model systems or are described to be expressed in neural cells (NeuroD2). Moreover, by searching similar gene combinations in the human and rodent genome additional homologous chromosomal regions on chromosome 3p21 and 12q13 harboring several isoforms of the respective genes (see below) were found. There was a strong evidence for multiple interactions between the 43 candidate genes, as being part of identical pathways (HER-2, neu, GRB7, CrkRS, CDC6), influencing the expression of each other (Her-2/neu, THRA, RARA), interacting with each other (PPARGBP, THRA, RARA, NR1D1 or Her-2/neu, GRB7) or expressed in defined tissues (CACNB1, PPARGBP, etc.). Interestingly, the genomic regions of the ARCHEONs that were identified are amplified in acquired Tamoxifen resistance of Her-2/neu negative cells (MCF7), which are normally sensitive to Tamoxifen treatment [Achuthan et al., 2001, (2)].

According to the observations described above the following examples of genes at 3q21-26 are offered by way of illustration, not by way of limitation.
[symbol] WNT5A, CACNA1D, THRB, RARB, TOP2B, RAB5B, SMARCC1 (BAF155), RAF, WNT7A

The following examples of genes at 12q13 are offered by way of illustration, not by way of limitation.
[symbol] CACNB3, Keratins, NR4A1, RAB5/13, RARgamma, STAT6, WNT10B, (GCN5), (SAS: Sarcoma Amplified Sequence), SMARCC2 (BAF170), SMARCD1 (BAF60A), (GAS41: Glioma Amplified Sequence), (CHOP), Her3, KRTHB, HOX C, IGFBP6, WNT5B

There is cross-talk between the amplified ARCHEONs described above and some other highly amplified genomic regions locate approximately at 7p12, 8q24 and 11q13. The above mentioned chromosomal regions are described by way of illustration not by way of limitation, as the amplified regions often span larger and/or overlapping positions at these chromosomal positions.

Another aspect of the present invention is based on the observation that neighboring genes within defined genomic regions functionally interact and influence each others function directly or indirectly. A genomic region encoding functionally interacting genes that are co-amplified and co-expressed in neoplastic lesions has been defined as an “ARCHEON”. (ARCHEON=Altered Region of Changed Chromosomal Expression Observed in Neoplasms). Chromosomal alterations often affect more than one gene. This is true for amplifications, duplications, insertions, integrations, inversions, translocations, and deletions. These changes can have influence on the expression level of single or multiple genes. Most commonly in the field of cancer diagnostics and treatment the changes of expression levels have been investigated for single, putative relevant target genes such as MLVI2 (5p14), NRASL3 (6p12), EGFR (7p12), c-myc (8q24), Cyclin D1 (11q13), IGF1R (15q25), Her-2/neu (17q12), PCNA (20q12). However, the altered expression level and interaction of multiple (i.e. more than two) genes within one or more genomic regions with each other has not been addressed. Moreover, the interaction of multiple genes of these genomic regions and their functional interplay with regard to prediction of response to treatment and outcome of therapy has not been analyzed. We have found that this is particularly informative with regard to response to chemotherapy and endocrine therapy. In addition we have found that the response to targeted therapy greatly depends on the constitutive expression of genes due to chromosomal alterations. The overexpression of genes by genomic amplification is frequent in early cancer development of multiple cancers and enables to stably acquire biological characteristics, that are of advantage for tumor growth including self sufficiency in growth signals, insensitivity to induction of apoptosis, limitless replicative potentials, tissue invasion and metastasis, sustained angiogenesis. However, as these molecular changes are stable, the cells become dependent on these characteristics and cannot turn the activities off in case of disadvantages due to targeted therapy approaches. Even more important, as these genomic changes can harbor biological characteristics being advantageous for tumor spread, they are often being maintained and present not only in the primary tumor but also in the metastatic leisons. By solely analyzing the mRNA or protein expression of target genes being present in such regions, one cannot determine the genomic status of the tumor, as these genes are often expressed without underlying genomic changes. However, we have found that tumors expressing these genes without underlying genomic changes can compensate for disadvantages by modulating the target gene expression and thereby escaping the toxic effect. Being even more important researchers have focussed on singular, well known gene members in such regions.

As an example, also depicted in the background of the invention, researchers from the NSABP Operations and Biostatistical Center focussed on c-myc and Her-2/neu itself, when interpreting FISH data pinpointing to a prominent role of the 8q24 chromosomal amplification for Her-2/neu positive tumors (as depicted by FISH analysis of 17q12), when being treated with trastuzumab. However, these analysis was done by determining DNA amplification status. The RNA expression levels of c-Myc and Her-2/neu have not been addressed, as it was not possible for them to analyze the RNA expression level in formalin fixed paraffin embedded tissues, which was the only tumor sample source. We have developed a methodology which enables such analysis even in tissues of low tumor content and low tissue amount. By analyzing the 17q12 and 8q24 in trastuzumab treated patients, we could prove, that the stable overexpression of the Her-2/neu receptor from chromosome 17q12 and the TRIB1 downstream target of the Her-2/neu/MAPK pathway from chromosome 8q24 is critical for the tumor to respond. This interaction of two genes within two different ARCHEONs, which are coamplified relatively frequently, proves our concept how to use ARCHEON gene analysis for prediction and prognosis of cancer.

Genes of an ARCHEON form gene clusters with tissue specific expression patterns. The mode of interaction of individual genes within such a gene cluster suspected to represent an ARCHEON can be either protein-protein or protein-nucleic acid interaction, which may be illustrated but not limited by the following examples: ARCHEON gene interaction may be in the same signal transduction pathway, may be receptor to ligand binding, receptor kinase and SH2 or SH3 binding, transcription factor to promoter binding, nuclear hormone receptor to transcription factor binding, phosphogroup donation (e.g. kinases) and acceptance (e.g. phosphoprotein), mRNA stabilizing protein binding and transcriptional processes. The individual activity and specificity of a pair genes and or the proteins encoded thereby or of a group of such in a higher order, may be readily deduced from literature, published or deposited within public databases by the skilled person. However in the context of an ARCHEON the interaction of members being part of an ARCHEON will potentiate, exaggerate or reduce their singular functions. This interaction is of importance in defined normal tissues in which they are normally co-expressed. Therefore, these clusters have been commonly conserved during evolution. The aberrant expression of members of these ARCHEON in neoplastic lesions, however, (especially within tissues in which they are normally not expressed) has influence on tumor characteristics such as growth, invasiveness and drug responsiveness. Due to the interaction of these neighboring genes it is of importance to determine the members of the ARCHEON which are involved in the deregulation events. In this regard amplification and deletion events in neoplastic lesions are of special interest.

In one embodiment the presence or absence of alterations of genes within distinct genomic regions are correlated with each other, as exemplified for breast cancer cell lines. This confers to the discovery of the present invention, that multiple interactions of said gene products of defined chromosomal localizations happen, that according to their respective alterations in abnormal tissue have predictive, diagnostic, prognostic and/or preventive and therapeutic value. These interactions are mediated directly or indirectly, due to the fact that the respective genes are part of interconnected or independent signaling networks or regulate cellular behavior (differentiation status, proliferative and/or apoptotic capacity, invasiveness, drug responsiveness, immune modulatory activities) in a synergistic, antagonistic or independent fashion.

There is cross-talk between the amplified ARCHEONs described above and some other highly amplified genomic regions locate approximately at 1p13, 1q32, 2p16, 2q21, 3p12, 5p13, 6p12, 7p12, 7q21, 8q23, 11q13, 13q12, 19q13, 20q13 and 21q11. The above mentioned chromosomal regions are described by way of illustration not by way of limitation, as the amplified regions often span larger and/or overlapping positions at these chromosomal positions. Genetic interactions within ARCHEONs

Genes involved in genomic alterations (amplifications, insertions, translocations, deletions, etc.) exhibit changes in their expression pattern. Of particular interest are gene amplifications, which account for gene copy numbers >2 per cell or deletions accounting for gene copy numbers <2 per cell. Gene copy number and gene expression of the respective genes do not necessarily correlate. Transcriptional overexpression needs an intact transcriptional context, as determined by regulatory regions at the chromosomal locus (promotor, enhancer and silencer), and sufficient amounts of transcriptional regulators being present in effective combinations. This is especially true for genomic regions, which expression is tightly regulated in specific tissues or during specific developmental stages. ARCHEONs are specified by gene clusters of two or more genes being directly neighbored or in chromosomal order, interspersed by a maximum of 10, preferably 7, more preferably 5 or at least 1 gene. The interspersed genes are also co-amplified but do not directly interact with the ARCHEON. Such an ARCHEON may spread over a chromosomal region of a maximum of 20, more preferably 10 or 5 Megabases, or contains at least two genes. The nature of an ARCHEON is characterized by the simultaneous amplification and/or deletion of the encompassed genes which results in upregulation or downregulation of specific genes within these regions. These expression patterns can also be found in a specific tissues, cell types, cellular or developmental states or time points and is of functional importance. Such ARCHEONs are commonly conserved during evolution, as they play critical roles during cellular development. In case of these ARCHEONs whole gene clusters are overexpressed upon amplification as they harbor self-regulatory feedback loops, which stabilize gene expression and/or biological effector function even in abnormal biological settings, or are regulated by very similar transcription factor combinations, reflecting their simultaneous function in specific tissues at certain developmental stages. Therefore, the gene copy numbers correlates with the expression level especially for genes in gene clusters functioning as ARCHEONs. In case of abnormal gene expressions in neoplastic lesions it is of great importance to know whether the self-regulatory feedback loops have been conserved as they determine the biological activity of the ARCHEON gene members.

The intensive interaction between genes in ARCHEONs confers to the discovery of the present invention, that multiple interactions of said gene products of defined chromosomal localizations happen, that according to their respective alterations in abnormal tissue have predictive, diagnostic, prognostic and/or preventive and therapeutic value. These interactions are mediated directly or indirectly, due to the fact that the respective genes are part of interconnected or independent signaling networks or regulate cellular behavior (differentiation status, proliferative and/or apoptotic capacity, invasiveness, drug responsiveness, immune modulatory activities) in a synergistic, antagonistic or independent fashion. It has been found that the co-amplification of genes within ARCHEONs can lead to co-expression of the respective gene products. Some of said genes also exhibit additional mutations or specific patterns of polymorphisms, which are substantial for the oncogenic capacities of these ARCHEONs. It is one of the critical features of such amplicons, which members of the ARCHEON have been conserved during tumor formation (e.g. during amplification and deletion events), thereby defining these genes as diagnostic marker genes. Moreover, the expression of the certain genes within the ARCHEON can be influenced by other members of the ARCHEON, thereby defining the regulatory and regulated genes as target genes for therapeutic intervention.

The invention also relates to the combinatorial analysis of genomic alterations as defined by discrete ARCHEON gene expressions together with the analysis of hormonal activities in the tumor. Interestingly, this correlates with feedback regulations between ARCHEON genes expression itself and ER and PR hormone receptor status. It is one finding, that the presence of hormone receptors (e.g. THRA, RARA within the 17q12 ARCHEON) and hormone receptor associated genes (e.g. PPARBP within the 17q12 ARCHEON) is relevant for prognosis and response to chemotherapy and antibody containing regimen. However, particularly in ER negative tumors, the hormone influence is less prominent resulting in less differentiated, higher grade tumors, which are sensitive to chemotherapy and antibody containing regimen. Therefore, it is important to address the hormonal status when analyzing genomically instable tumors.

The invention relates to a method for the detection of chromosomal alterations by (a) determining the relative mRNA abundance of individual mRNA species or (b) determining the copy number of one or more chromosomal region(s) by quantitative PCR. In one embodiment information on the genomic organization and spatial regulation of chromosomal regions is assessed by bioinformatic analysis of the sequence information of the human genome (UCSC, NCBI) and then combined with RNA expression data from GeneChip™ DNA-Arrays (Affymetrix) and/or quantitative PCR (TaqMan) from RNA-samples or genomic DNA.

The present invention further relates to the simultaneous analysis of RNA expression and DNA alteration within identical tissue samples or nucleic acid extractions, as e.g. the combinatorial analysis of RNA expression level on basis of DNA amplification status harbours additional and new information, which cannot be provided by solely analyzing RNA od DNA status of the respective genes.

The present invention further relates to a method for the detection of chromosomal alterations characterized in that the copy number of one or more genomic nucleic acid sequences located within an altered chromosomal region(s) is detected by quantitative PCR techniques (e.g. TaqMan™, Lightcycler™ and iCycler™).

The present invention further relates to methods for detecting these deregulations in malignant neoplasia on DNA and mRNA level.

The present invention further relates to a method for the prediction, diagnosis or prognosis of malignant neoplasia by the detection of at least 2 markers whereby the markers are genes and fragments thereof or genomic nucleic acid sequences that are located on one chromosomal region which is altered in malignant neoplasia and breast cancer in particular. In particular not only the intragenic regions, but also intergenic regions, pseudogenes or non-transcribed genes of said chromosomal regions can be used for diagnostic, predictive, prognostic and preventive and therapeutic compositions and methods.

The present invention also discloses a method for the prediction, diagnosis or prognosis of malignant neoplasia by the detection of at least 2 markers whereby the markers are located on one or more chromosomal region(s) which is/are altered in malignant neoplasia; and the markers interact as (i) receptor and ligand or (ii) members of the same signal transduction pathway or (iii) members of synergistic signal transduction pathways or (iv) members of antagonistic signal transduction pathways or (v) transcription factor and transcription factor binding site.

In another embodiment the expression of these genes can be detected with DNA-arrays as described in WO9727317 and U.S. Pat. No. 6,379,895.

In a further embodiment the expression of these genes can be detected with bead based direct fluorescent readout techniques such as described in WO9714028 and WO9952708.

The present invention further relates to a method for the detection of chromosomal alterations characterized in that the relative abundance of individual mRNAs, encoded by genes, located in altered chromosomal regions is detected.

The present invention further relates to a method for the detection of the flanking breakpoints of named chromosomal alterations by measurement of DNA copy number by quantitative PCR or DNA-Arrays and DNA sequencing.

### Biological Functions of Said Genes

### DEFINITIONS

The term “marker” or “biomarker” refers a biological molecule, e.g., a nucleic acid, peptide, hormone, etc., whose presence or concentration can be detected and correlated with a known condition, such as a disease state.

“Marker gene,” as used herein, refers to a differentially expressed gene which expression pattern may be utilized as part of predictive, prognostic or diagnostic malignant neoplasia or breast cancer evaluation, or which, alternatively, may be used in methods for identifying compounds useful for the treatment or prevention of malignant neoplasia and breast cancer in particular. A marker gene may also have the characteristics of a target gene.

“Target gene”, as used herein, refers to a differentially expressed gene involved in breast cancer in a manner by which modulation of the level of target gene expression or of target gene product activity may act to ameliorate symptoms of malignant neoplasia and breast cancer in particular. A target gene may also have the characteristics of a marker gene.

The term “altered chromosomal region” or “abberant chromosomal region” refers to a structural change of the chromosomal composition and DNA sequence, which can occur by the following events: amplifications, deletions, inversions, insertions, translocations and/or viral integrations. A trisomy, where a given cell harbors more than two copies of a chromosome, is within the meaning of the term “amplification” of a chromosome or chromosomal region.

“Differential expression”, or “expression” as used herein, refers to both quantitative as well as qualitative differences in the genes' expression patterns observed in at least two different individuals or samples taken from individuals. Differential expression may depend on differential development, different genetic background of tumor cells and/or reaction to the tissue environment of the tumor. Differentially expressed genes may represent “marker genes,” and/or “target genes”. The expression pattern of a differentially expressed gene disclosed herein may be utilized as part of a prognostic or diagnostic cancer evaluation.

The term “pattern of expression” refers, e.g., to a determined level of gene expression compared either to a reference gene (e.g. housekeeper) or to a computed average expression value (e.g. in DNA-chip analyses). A pattern is not limited to the comparison of two genes but even more related to multiple comparisons of genes to a reference genes or samples. A certain “pattern of expression” may also result and be determined by comparison and measurement of several genes disclosed hereafter and display the relative abundance of these transcripts to each other.

Alternatively, a differentially expressed gene disclosed herein may be used in methods for identifying reagents and compounds and uses of these reagents and compounds for the treatment of cancer as well as methods of treatment. The differential regulation of the gene is not limited to a specific cancer cell type or clone, but rather displays the interplay of cancer cells, muscle cells, stromal cells, connective tissue cells, other epithelial cells, endothelial cells and blood vessels as well as cells of the immune system (e.g. lymphocytes, macrophages, killer cells).

A “reference pattern of expression levels”, within the meaning of the invention shall be understood as being any pattern of expression levels that can be used for the comparison to another pattern of expression levels. In a preferred embodiment of the invention, a reference pattern of expression levels is, e.g., an average pattern of expression levels observed in a group of healthy or diseased individuals, serving as a reference group.

“Primer pairs and probes”, within the meaning of the invention, shall have the ordinary meaning of this term which is well known to the person skilled in the art of molecular biology. In a preferred embodiment of the invention “primer pairs and probes”, shall be understood as being polynucleotide molecules having a sequence identical, complementary, homologous, or homologous to the complement of regions of a target polynucleotide which is to be detected or quantified.

“Individually labeled probes”, within the meaning of the invention, shall be understood as being molecular probes comprising a polynucleotide or oligonucleotide and a label, helpful in the detection or quantification of the probe. Preferred labels are fluorescent labels, luminescent labels, radioactive labels and dyes.

“Arrayed probes”, within the meaning of the invention, shall be understood as being a collection of immobilized probes, preferably in an orderly arrangement. In a preferred embodiment of the invention, the individual “arrayed probes” can be identified by their respective position on the solid support, e.g., on a “chip”.

The phrase “tumor response”, “therapeutic success”, or “response to therapy” refers, in the therapeutic setting to the observation of a reduction in tumor mass (as specified by WHQ or RECIST Criteria) defined tumor free, recurrence free or overall survival time (e.g. 2 years, 4 years, 5 years, 10 years). This time period of disease free survival may vary among the different tumor entities but is sufficiently longer than the average time period in which most of the recurrences appear. In a neoadjuvant therapy modality response may be monitored by measurement of tumor shrinkage due to apoptosis and necrosis of the tumor mass.

The term “recurrence” or “recurrent disease” does include distant metastasis that can appear even many years after the initial diagnosis and therapy of a tumor, or to local events such as infiltration of tumor cell into regional lymph nodes, or occurrence of tumor cells at the same site and organ of origin within an appropriate time.

“Prediction of recurrence” or “prediction of success” does refer to the methods an compositions described in this invention. Wherein a tumor specimen is analyzed for it's gene expression and furthermore classified based on correlation of the expression pattern to known ones from reference samples. This classification may either result in the statement that such given tumor will develop recurrence and therefore is considered as a “non responding” tumor to the given therapy, or may result in a classification as a tumor with a prorogued disease free post therapy time.

“Discriminant function analysis” is a technique used to determine which variables discriminate between two or more naturally occurring mutually exclusive groups. The basic idea underlying discriminant function analysis is to determine whether groups differ with regard to a set of predictor variables which may or may not be independent of each other, and then to use those variables to predict group membership (e.g., of new cases).

Discriminant function analysis starts with an outcome variable that is categorical (two or more mutually exclusive levels). The model assumes that these levels can be discriminated by a set of predictor variables which, like ANOVA (analysis of variance), can be continuous or categorical (but are preferably continuous) and, like ANOVA assumes that the underlying discriminant functions are linear. Discriminant analysis does not “partition variation”. It does look for canonical correlations among the set of predictor variables and uses these correlates to build eigenfunctions that explain percentages of the total variation of all predictor variables over all levels of the outcome variable.

The output of the analysis is a set of linear discriminant functions (eigenfunctions) that use combinations of the predictor variables to generate a “discriminant score” regardless of the level of the outcome variable. The percentage of total variation is presented for each function. In addition, for each eigenfunction, a set of Fisher Discriminant Functions are developed that produce a discriminant score based on combinations of the predictor variables within each level of the outcome variable.

Usually, several variables are included in a study in order to see which variable contribute to the discrimination between groups. In that case, a matrix of total variances and co-variances is generated. Similarly, a matrix of pooled within-group variances and co-variances may be generated. A comparison of those two matrices via multivariate F tests is made in order to determine whether or not there are any significant differences (with regard to all variables) between groups. This procedure is identical to multivariate analysis of variance or MANOVA. As in MANOVA, one could first perform the multivariate test, and, if statistically significant, proceed to see which of the variables have significantly different means across the groups.

For a set of observations containing one or more quantitative variables and a classification variable defining groups of observations, the discrimination procedure develops a discriminant criterion to classify each observation into one of the groups. In order to get an idea of how well a discriminant criterion “performs”, it is necessary to classify (a priori) different cases, that is, cases that were not used to estimate the discriminant criterion. Only the classification of new cases enables an assessment of the predictive validity of the discriminant criterion.

In order to validate the derived criterion, the classification can be applied to other data sets. The data set used to derive the discriminant criterion is called the training or calibration data set or patient training cohort. The data set used to validate the performance of the discriminant criteria is called the validation data set or validation cohort.

The discriminant criterion (function(s) or algorithm), determines a measure of generalized squared distance. These distances are based on the pooled co-variance matrix. Either Mahalanobis or Euclidean distance can be used to determine proximity. These distances can be used to identify groupings of the outcome levels and so determine a possible reduction of levels for the variable.

A “pooled co-variance matrix” is a numerical matrix formed by adding together the components of the covariance matrix for each subpopulation in an analysis.

A “predictor” is any variable that may be applied to a function to generate a dependent or response variable or a “predictor value”. In one embodiment of the instant invention, a predictor value may be a discriminant score determined through discriminant function analysis of two or more patient blood markers (e.g., plasma or serum markers). For example, a linear model specifies the (linear) relationship between a dependent (or response) variable Y, and a set of predictor variables, the X′s, so that

Y=b₀+b₁X₁+b₂X₂+ . . . +bₖXₖ

In this equation b₀ is the regression coefficient for the intercept and the bᵢ values are the regression coefficients (for variables 1 through k) computed from the data.

“Classification trees” are used to predict membership of cases or objects in the classes of a categorical dependent variable from their measurements on one or more predictor variables. Classification tree analysis is one of the main techniques used in so-called Data Mining. The goal of classification trees is to predict or explain responses on a categorical dependent variable, and as such, the available techniques have much in common with the techniques used in the more traditional methods of Discriminant Analysis, Cluster Analysis, Nonparametric Statistics, and Nonlinear Estimation.

The flexibility of classification trees makes them a very attractive analysis option, but this is not to say that their use is recommended to the exclusion of more traditional methods. Indeed, when the typically more stringent theoretical and distributional assumptions of more traditional methods are met, the traditional methods may be preferable. But as an exploratory technique, or as a technique of last resort when traditional methods fail, classification trees are, in the opinion of many researchers, unsurpassed. Classification trees are widely used in applied fields as diverse as medicine (diagnosis), computer science (data structures), botany (classification), and psychology (decision theory). Classification trees readily lend themselves to being displayed graphically, helping to make them easier to interpret than they would be if only a strict numerical interpretation were possible.

“Neural Networks” are analytic techniques modeled after the (hypothesized) processes of learning in the cognitive system and the neurological functions of the brain and capable of predicting new observations (on specific variables) from other observations (on the same or other variables) after executing a process of so-called learning from existing data. Neural Networks is one of the Data Mining techniques. The first step is to design a specific network architecture (that includes a specific number of “layers” each consisting of a certain number of “neurons”). The size and structure of the network needs to match the nature (e.g., the formal complexity) of the investigated phenomenon. Because the latter is obviously not known very well at this early stage, this task is not easy and often involves multiple “trials and errors.”

The neural network is then subjected to the process of “training.” In that phase, computer memory acts as neurons that apply an iterative process to the number of inputs (variables) to adjust the weights of the network in order to optimally predict the sample data on which the “training” is performed. After the phase of learning from an existing data set, the new network is ready and it can then be used to generate predictions.

In one embodiment of the invention, neural networks can comprise memories of one or more personal or mainframe computers or computerized point of care device.

“Cox Regression Analysis” is a statistical technique whereby Cox proportional-hazards regression is used to analyze the effect of several risk factors on survival. The probability of the endpoint (death, or any other event of interest, e.g. recurrence of disease) is called the hazard. The hazard is modeled as:

H(t)=H₀(t)×exp(b₁X₁+b₂X₂+b₃X₃+ . . . +bₖXₖ)

where X₁ . . . Xₖ are a collection of predictor variables and H₀(t) is the baseline hazard at time t, representing the hazard for a person with the value 0 for all the predictor variables.

By dividing both sides of the above equation by H₀(t) and taking logarithms, we obtain:

$$ln ( \frac{H ( t )}{{H}_{0} ( t )} ) = {b}_{1} {X}_{1} + {b}_{2} {X}_{2} + {b}_{3} {X}_{3} + … + {b}_{k} {X}_{k}$$

H(t)/H₀(t) is the hazard ratio. The coefficients bᵢ . . . bₖ are estimated by Cox regression, and can be interpreted in a similar manner to that of multiple logistic regression.

If the covariate (risk factor) is dichotomous and is coded 1 if present and 0 if absent, then the quantity exp(bᵢ) can be interpreted as the instantaneous relative risk of an event, at any time, for an individual with the risk factor present compared with an individual with the risk factor absent, given both individuals are the same on all other covariates. If the covariate is continuous, then the quantity exp(bᵢ) is the instantaneous relative risk of an event, at any time, for an individual with an increase of 1 in the value of the covariate compared with another individual, given both individuals are the same on all other covariates.

“Kaplan Meier curves” are a nonparametric (actuarial) technique for estimating time-related events (the survivorship function). 1 Ordinarily, Kaplan Meier curves are used to analyze death as an outcome. It may be used effectively to analyze time to an endpoint, such as remission. Kaplan Meier curves are a univariate analysis, an appropriate starting technique, and estimate the probability of the proportion of individuals in remission at a particular time, starting from the initiation of active date (time zero), is especially applicable when length of follow-up varies from patient to patient, and takes into account those patients lost during follow-up or not yet in remission at end of a clinical study (e.g., censored patients, where the censoring is non-informative). Kaplan Meier is therefore useful in evaluating remissions following loosing a patient. Since the estimated survival distribution for the cohort study has some degree of uncertainty, 95% confidence intervals may be calculated for each survival probability on the “estimated” curve.

A variety of tests (log-rank, Wilcoxan and Gehen) may be used to compare two or more Kaplan-Meier “curves” under certain well-defined circumstances. Median remission time (the time when 50% of the cohort has reached remission), as well as quantities such as three, five, and ten year probability of remission, can also be generated from the Kaplan-Meier analysis, provided there has been sufficient follow-up of patients.

Kaplan-Meier and Cox regression analysis can be performed by using commercially available software packages, e.g., Graph Pad Prism® and SPSS versionII.

“Receiver Operator Characteristic Curve” (“ROC”): is a graphical representation of the functional relationship between the distribution of a marker's sensitivity and 1-specificity values in a cohort of diseased persons and in a cohort of non-diseased persons.

“Area Under the Curve” (“AUC”) is a number which represents the area under a Receiver Operator Characteristic curve. The closer this number is to one, the more the marker values discriminate between diseased and non-diseased cohorts

“McNemar Chi-square Test” (“The McNemar χ² test”) is a statistical test used to determine if two correlated proportions (proportions that share a common numerator but different denominators) are significantly different from each other.

A “nonparametric regression analysis” is a set of statistical techniques that allows the fitting of a line for bivariate data that make little or no assumptions concerning the distribution of each variable or the error in estimation of each variable. Examples are: Theil estimators of location, Passing-Bablok regression, and Deming regression.

“Cut-off values” or “Threshold values” are numerical value of a marker (or set of markers) that defines a specified sensitivity or specificity.

“Biological activity” or “bioactivity” or “activity” or “biological function”, which are used inter-changeably, herein mean an effector or antigenic function that is directly or indirectly performed by a polypeptide (whether in its native or denatured conformation), or by any fragment thereof in vivo or in vitro. Biological activities include but are not limited to binding to polypeptides, binding to other proteins or molecules, enzymatic activity, signal transduction, activity as a DNA binding protein, as a transcription regulator, ability to bind damaged DNA, etc. A bioactivity can be modulated by directly affecting the subject polypeptide. Alternatively, a bioactivity can be altered by modulating the level of the polypeptide, such as by modulating expression of the corresponding gene.

The term “marker” or “biomarker” refers a biological molecule, e.g., a nucleic acid, peptide, hormone, etc., whose presence or concentration can be detected and correlated with a known condition, such as a disease state.

The term “marker gene,” as used herein, refers to a differentially expressed gene which expression pattern may be utilized as part of predictive, prognostic or diagnostic process in malignant neoplasia or cancer evaluation, or which, alternatively, may be used in methods for identifying compounds useful for the treatment or prevention of malignant neoplasia and lung, ovarian, cervix, head and neck, stomach, pancreas, colon or breast cancer in particular. A marker gene may also have the characteristics of a target gene.

“Target gene”, as used herein, refers to a differentially expressed gene involved in ovarian, cervix, stomach, pancreas, head and neck, colon or breast cancer in a manner by which modulation of the level of target gene expression or of target gene product activity may act to ameliorate symptoms of malignant neoplasia and lung, ovarian, cervix, head and neck, stomach, pancreas, colon or breast cancer in particular. A target gene may also have the characteristics of a marker gene.

The term “neoplastic lesion” or “neoplastic disease” or “neoplasia” refers to a cancerous tissue this includes carcinomas, (e.g., carcinoma in situ, invasive carcinoma, metastatic carcinoma) and pre-malignant conditions, neomorphic changes independent of their histological origin (e.g. ductal, lobular, medullary, mixed origin). The term “cancer” is not limited to any stage, grade, histomorphological feature, invasiveness, agressivity or malignancy of an affected tissue or cell aggregation. In particular stage 0 cancer, stage I cancer, stage II cancer, stage III cancer, stage IV cancer, grade I cancer, grade II cancer, grade III cancer, malignant cancer, primary carcinomas, and all other types of cancers, malignancies and transformations associated with the lung, ovary, cervix, head and neck, stomach, pancreas, colon or breast are included. The terms “neoplastic lesion” or “neoplastic disease” or “neoplasia” or “cancer” are not limited to any tissue or cell type they also include primary, secondary or metastatic lesion of cancer patients, and also comprises lymph nodes affected by cancer cells or minimal residual disease cells either locally deposited (e.g. bone marrow, liver, kidney, brain) or freely floating throughout the patients body.

Furthermore, the term “characterizing the sate of a neoplastic disease” is related to, but not limited to, measurements and assessment of one or more of the following conditions: Type of tumor, histomorphological appearance, dependence on external signal (e.g. hormones, growth factors), invasiveness, motility, state by TNM (2) or similar, agressivity, malignancy, metastatic potential, and responsiveness to a given therapy.

The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, cell-containing body fluids, free floating nucleic acids, urine, stool, peritoneal fluid, and pleural fluid, or cells therefrom. Biological samples may also include sections of tissues such as frozen or fixed sections taken for histological purposes. A biological sample to be analyzed is tissue material from neoplastic lesion taken by aspiration or punctuation, excision or by any other surgical method leading to biopsy or resected cellular material. Such biological sample may comprises cells obtained from a patient. The cells may be found in a cell “smear” collected, for example, by a nipple aspiration, ductal lavarge, fine needle biopsy or from provoked or spontaneous nipple discharge. In another embodiment, the sample is a body fluid. Such fluids include, for example, blood fluids, lymph, ascitic fluids, gynecological fluids, or urine but not limited to these fluids.

The term “therapy modality”, “therapy mode”, “regimen” or “chemo regimen” as well as “therapy regime” refers to a timely sequential or simultaneous administration of anti tumor, and/or immune stimulating, and/or blood cell proliferative agents, and/or radiation therapy, and/or hyperthermia, and/or hypothermia for cancer therapy. The administration of these can be performed in an adjuvant and/or neoadjuvant mode. The composition of such “protocol” may vary in dose of the single agent, timeframe of application and frequency of administration within a defined therapy window. Currently various combinations of various drugs and/or physical methods, and various schedules are under investigation.

By “array” or “matrix” is meant an arrangement of addressable locations or “addresses” on a device. The locations can be arranged in two dimensional arrays, three dimensional arrays, or other matrix formats. The number of locations can range from several to at least hundreds of thousands. Most importantly, each location represents a totally independent reaction site. Arrays include but are not limited to nucleic acid arrays, protein arrays and antibody arrays. A “nucleic acid array” refers to an array containing nucleic acid probes, such as oligonucleotides, polynucleotides or larger portions of genes. The nucleic acid on the array is preferably single stranded. Arrays wherein the probes are oligonucleotides are referred to as “oligonucleotide arrays” or “oligonucleotide chips.” A “microarray,” herein also refers to a “biochip” or “biological chip”, an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. A “protein array” refers to an array containing polypeptide probes or protein probes which can be in native form or denatured. An “antibody array” refers to an array containing antibodies which include but are not limited to monoclonal antibodies (e.g. from a mouse), chimeric antibodies, humanized antibodies or phage antibodies and single chain antibodies as well as fragments from antibodies.

The term “agonist”, as used herein, is meant to refer to an agent that mimics or upregulates (e.g., potentiates or supplements) the bioactivity of a protein. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide with another molecule, e.g., a target peptide or nucleic acid.

The term “antagonist” as used herein is meant to refer to an agent that downregulates (e.g., suppresses or inhibits) at least one bioactivity of a protein. An antagonist can be a compound which inhibits or decreases the interaction between a protein and another molecule, e.g., a target peptide, a ligand or an enzyme substrate. An antagonist can also be a compound that down-regulates expression of a gene or which reduces the amount of expressed protein present.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

The terms “modulated” or “modulation” or “regulated” or “regulation” and “differentially regulated” as used herein refer to both upregulation (i.e., activation or stimulation (e.g., by agonizing or potentiating) and down regulation [i.e., inhibition or suppression (e.g., by antagonizing, decreasing or inhibiting)].

“Transcriptional regulatory unit” refers to DNA sequences, such as initiation signals, enhancers, and promoters, which induce or control transcription of protein coding sequences with which they are operably linked. In preferred embodiments, transcription of one of the genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally occurring forms of the polypeptide.

The term “derivative” refers to the chemical modification of a polypeptide sequence, or a polynucleotide sequence. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived. The term “derivative” furthermore refers to phosphorylated forms of a polypeptide sequence or protein.

The term “nucleotide analog” refers to oligomers or polymers being at least in one feature different from naturally occurring nucleotides, oligonucleotides or polynucleotides, but exhibiting functional features of the respective naturally occurring nucleotides (e.g. base paring, hybridization, coding information) and that can be used for said compositions. The nucleotide analogs can consist of non-naturally occurring bases or polymer backbones, examples of which are LNAs, PNAs and Morpholinos. The nucleotide analog has at least one molecule different from its naturally occurring counterpart or equivalent.

The term “equivalent”, with respect to a nucleotide sequence, is understood to include nucleotide sequences encoding functionally equivalent polypeptides. Equivalent nucleotide sequences will include sequences that differ by one or more nucleotide substitutions, additions or deletions, such as allelic variants and therefore include sequences that differ due to the degeneracy of the genetic code. “Equivalent” also is used to refer to amino acid sequences that are functionally equivalent to the amino acid sequence of a mammalian homolog of a marker protein, but which have different amino acid sequences, e.g., at least one, but fewer than 30, 20, 10, 7, 5, or 3 differences, e.g., substitutions, additions, or deletions.

“Homology”, “homologs of”, “homologous”, or “identity” or “similarity” refers to sequence similarity between two polypeptides or between two nucleic acid molecules, with identity being a more strict comparison. Homology and identity can each be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences. Other techniques for determining sequence identity are well-known and described in the art. Preferred nucleic acids used in the instant invention have a sequence at least 70%, and more preferably 80% identical and more preferably 90% and even more preferably at least 95% identical to, or complementary to, a nucleic acid sequence of a mammalian homolog of a gene that expresses a marker as defined previously. Particularly preferred nucleic acids used in the instant invention have a sequence at least 70%, and more preferably 80% identical and more preferably 90% and even more preferably at least 95% identical to, or complementary to, a nucleic acid sequence of a mammalian homolog of a gene that expresses a marker as defined previously.

“Prognostic Markers” as used herein refers to factors, that provide information about the clinical outcome of patients with or without treatment. The information provided by prognostic markers is not affected by therapeutic interference.

“Predictive Markers” as used herein refers to factors, that provide information about the possible response of a tumor to a distinct therapeutic agent or regimen

The term “marker” or “biomarker” refers a biological molecule, e.g., a nucleic acid, peptide, hormone, etc., whose presence or concentration can be detected and correlated with a known condition, such as a disease state.

Staging is a method to describe how advanced a cancer is. Staging for colorectal cancer takes into account the depth of invasion into the colon wall, and spread to lymph nodes and other organs.

Stage 0 (Carcinoma in Situ): Stage 0 cancer is also called carcinoma in situ. This is a precancerous condition, usually found in a polyp. Stage I (Dukes A): The cancer has spread through the innermost lining of the colon to the second and third layers of the colon wall. It has not spread outside the colon. Stage II (Dukes B): The cancer has spread through the colon wall outside the colon to nearby tissues. Stage III (Dukes C): Cancer has spread to nearby lymph nodes, but not to other parts of the body. Stage IV: Cancer has spread to other parts of the body, e.g. metastasized to the liver or lungs.

“CANCER GENES” or “CANCER GENE” as used herein refers to the polynucleotides Table 1 and Ib, as well as derivatives, fragments, analogs and homologues thereof, the polypeptides encoded thereby as well as derivatives, fragments, analogs and homologues thereof and the corresponding genomic transcription units which can be derived or identified with standard techniques well known in the art using the information disclosed in Tables 1 and 1b. The Gene symbol, Gene Description, Reference sequence, Unigene ID, and OMIM number are shown in Tables 1a and 1b.

The term “kif” as used herein refers to any manufacture (e.g. a diagnostic or research product) comprising at least one reagent, e.g. a probe, for specifically detecting the expression of at least one marker gene disclosed in the invention, in particular of those genes listed in Tables 1a and 1b, whereas the manufacture is being sold, distributed, and/or promoted as a unit for performing the methods of the present invention. Also reagents (e.g. immunoassays) to detect the presence, the stability, activity, complexity of the respective marker gene products comprising polypeptides encoded by the genes listed in Tables 1a and 1b regard as components of the kit. In addition, any combination of nucleic acid and protein detection as disclosed in the invention are regard as a kit.

The present invention provides polynucleotide sequences and proteins encoded thereby, as well as probes derived from the polynucleotide sequences, antibodies directed to the encoded proteins, and predictive, preventive, diagnostic, prognostic and therapeutic uses for individuals which are at risk for or which have malignant neoplasia and lung, ovarian, pancreas, head and neck, stomach, pancreas, colon or breast cancer in particular. The sequences disclosure herein have been found to be differentially expressed in samples from head and neck, colon and breast cancer.

The present invention is based on the identification of 48 genes that are differentially regulated (up- or down regulated) in tumor biopsies of patients with clinical evidence of head and neck, colon and breast cancer. The combined analysis and characterization of the co-expression and interaction of these genes provides newly identified roles for disease outcome. Moreover 4 of these genes are targets of anti-cancer regimen. The detailed analysis of these genes thereby not only provides prognostic information, but also offers possibilities for risk adapted and individualized treatment options.

It is obvious to the person skilled in the art that a reference to a nucleotide sequence is meant to comprise the reference to the associated protein sequence which is coded by said nucleotide sequence.

“% identity” of a first sequence towards a second sequence, within the meaning of the invention, means the % identity which is calculated as follows: First the optimal global alignment between the two sequences is determined with the CLUSTALW algorithm [Thomson J D, Higgins D G, Gibson T J. 1994. ClustalW: Improving the sensitivity of progressive multiple sequence alignment through sequence weighting, positions-specific gap penalties and weight matrix choice. Nucleic Acids Res., 22: 4673-4680], Version 1.8, applying the following command line syntax: ./clustalw-infile=./infile.txt-output=-outorder=aligned-pwmatrix=gonnet-pwdnamatrix=clustalw-pwgapopen=10.0-pwgapext=0.1-matrix=gonnet-gapopen=10.0-gapext=0.05-gapdist=8-hgapresidues=GPSNDQERK-maxdiv=40. Implementations of the CLUSTAL W algorithm are readily available at numerous sites on the internet, including, e.g., http://www.ebi.ac.uk. Thereafter, the number of matches in the alignment is determined by counting the number of identical nucleotides (or amino acid residues) in aligned positions. Finally, the total number of matches is divided by the number of nucleotides (or amino acid residues) of the longer of the two sequences, and multiplied by 100 to yield the % identity of the first sequence towards the second sequence.

The present invention relates to:
1. A method for predicting therapeutic success of a given mode of treatment in a subject having cancer, comprising
   (i) determining the pattern of expression levels of at 1, 2, 3, 4, 5, 10, 15, 20, 25, 30, 35, 40, 50, 60 70 or 85 marker genes, comprised in the group of marker genes listed in Table 1,
   (ii) comparing the pattern of expression levels determined in (i) with one or several reference pattern(s) of expression levels,
   (iii) predicting therapeutic success for said given mode of treatment in said subject from the outcome of the comparison in step (ii).
2. A method for adapting therapeutic regimen based on individualized risk assessment for a subject having cancer, comprising
   (i) determining the pattern of expression levels of at least 1, 2, 3, 4, 5, 10, 15, 20, 25, 30, 35, 40, 50, 60 70 or 85 marker genes, comprised in the group of marker genes listed in Table 1,
   (ii) comparing the pattern of expression levels determined in (i) with one or several reference pattern(s) of expression levels,
   (iii) implementing therapeutic regimen targeting said marker genes in said subject from the outcome of the comparison in step (ii).
3. A method of count 1, wherein said given mode of treatment
   (i) acts on recruitment of lymphatic vessels
   (ii) acts on cell proliferation, and/or
   (iii) acts on cellular differentiation
   (iv) acts on cell motility; and/or
   (v) acts on cell survival, and/or
   (vi) acts on cellular metabolism
   (vii) acts on detoxification
   (viii) comprises administration of a chemotherapeutic agent
4. A method of count 1, 2 or 3, wherein said given mode of treatment comprises chemotherapy (5-FU based, anthracycline based, taxol based), small molecule inhibitors (Iressa, Sorafenib, SU 11248), antibody based regimen (Trastuzumab, avastin), anti-proliferation regimen, pro-apoptotic regimen, pro-differentiation regimen, radiation and surgical therapy.
5. A method of any of counts 1 to 3, wherein a predictive algorithm is used.
6. A method of treatment of a neoplastic disease in a subject, comprising
   (i) predicting therapeutic success for a given mode of treatment in a subject having cancer by the method of any of counts 1 to 4,
   (ii) treating said neoplastic disease in said patient by said mode of treatment, if said mode of treatment is predicted to be successful.
7. A method of selecting a therapy modality for a subject afflicted with a neoplastic disease, comprising
   (i) obtaining a biological sample from said subject,
   (ii) predicting from said sample, by the method of any of counts 1 to 4, therapeutic success in a subject having cancer for a plurality of individual modes of treatment,
   (iii) selecting a mode of treatment which is predicted to be successful in step (ii).
8. A method of any of counts 1 to 6, wherein the expression level is determined
   (i) with a hybridization based method, or
   (ii) with a hybridization based method utilizing arrayed probes, or
   (iii) with a hybridization based method utilizing individually labeled probes, or
   (iv) by real time real time PCR, or
   (v) by assessing the expression of polypeptides, proteins or derivatives thereof, or
   (vi) by assessing the amount of polypeptides, proteins or derivatives thereof.
9. A kit comprising at least 1, 2, 3, 4, 5, 10, 15, 20, 25, 30, 35, 40, 50, 60 70 or 85 primer pairs and probes suitable for marker genes comprised in the group of marker genes listed in Tables 1a and 1b.
10. A kit comprising at least 1 1, 2, 3, 4, 5, 10, 15, 20, 25, 30, 35, 40, 50, 60 70 or 85 individually labeled probes, each having a sequence complementary to any of sequences listed in Tables 1a and 1b.
11. A kit comprising at 1, 2, 3, 4, 5, 10, 15, 20, 25, 30, 35, 40, 50, 60 70 or 85 arrayed probes, each having a sequence complementary to any of the sequences listed in Tables 1a and 1b.

### EXAMPLE 1

### Summary

A statistically significant discrimination of tumor response (p less than about 0.05 level) was achieved using methods of the invention. Elevated or decreased levels of candidate gene expression and gene copy number were compared with normal control levels or adjusted mean levels of diseased cohorts. The significance of individual markers was determined by distinguishing tumor response parameters i.e. pathological complete response and lymphnode negativity after neoadjuvant chemotherapy, which will translate in differences in disease free and overall survival within this cohort. Calculation of the Kaplan-Meier plots from other patients receiving the combined chemo and antibody therapy in adjuvant and neoadjuvant situation (using the upper or lower quartile of the individual marker levels) demonstrates the clinical utility of the assessed markers. A decrease or increase in the levels of the markers in the cancer patient compared to the levels in normal controls indicated an increase in stage, grade, severity, advancement or progression of the patient's cancer and/or a lack of efficacy or benefit of the cancer treatment or therapy. In particular, high levels of TRIB1, Her-2/neu, MGC9753, c-Myc and low level of ER, PR correlated with good response to treatment with combined antibody and chemotherapy. In addition combined RNA and DNA analysis of CCND1, FGF factors and other genes being present on the 17q12, 8q24 and 11q13 ARCHEONs improved the prediction and prognosis of outcome compared to standard FISH technology approaches. Some singular serum parameters yielded statistically significant mean values and differentiated the cohorts according to differences in the study endpoints

### Clinical Methodology

Before treatment core-needle biopsies were taken from breast cancer patients (≧cT2, N0/N1, M0).

Thereafter and according to the clinical trial protocols eligible breast cancer patients received neoadjuvant chemotherapy of 4 cycles of epirubicin and cyclophosphamide (90/600 mg/m²) followed by 4 cycles paclitaxel (175 mg/m²). Trastuzumab was administered parallel to paclitaxel therapy on a three weekly dose (6 mg/kg) and continued for 36 weeks after surgery (according to the TECHNO trial) if tumors were IHC 3+ or FISH positive (=“EC-TH” regimen) Patients with Her-2 negative tumors (equally to IHC1+ or FISH negative testing) were not treated with trastuzumab (PREPARE trial) and served as controls. The Her-2 status was determined in pre-treatment, core-needle biopsies of all patients by immunohistochemistry or FISH analysis at a central reference pathology department. In total 853 paraffin embedded core needle biopsies are available for analysis. Up to 20 sections of 10 μm thickness were prepared from all tissues for further analysis. Tumor cell content and histology was centrally determined from a HE stained reference slide. DNA and RNA was successfully isolated from all tissues by an automated system based on magnetic beads (Bayer HealthCare Diagnostics). For comparison with Her-2/neu IHC and FISH data, the DNA and RNA extracted from whole tissue sections (i.e. without applying any microdissection) was analyzed by TaqMan PCR for Her-2/neu and neighbouring genes of the 17q12 ARCHEON (“Amplified Region of Chromosomal Expression Observed in Neoplasia”), that are also overexpressed due to the genomic amplification of this region.

### Experimental Methodology

Expression and Genomic Profiling Utilizing Quantitative PCR after Extraction of RNA and DNA from FFPE Core Needle Biopsies

For a detailed analysis of gene expression by quantitative PCR methods, one will utilize primers flanking the genomic region of interest and a fluorescent labeled probe hybridizing in-between. Using the PRISM 7900 Sequence Detection System of PE Applied Biosystems (Perkin Elmer, Foster City, Calif., USA) with the technique of a fluorogenic probe, consisting of an oligonucleotide labeled with both a fluorescent reporter dye and a quencher dye, one can perform such a expression measurement. Amplification of the probe-specific product causes cleavage of the probe, generating an increase in reporter fluorescence. Primers and probes were selected using the Primer Express software and localized mostly in the 3′ region of the coding sequence or in the 3′ untranslated region according to the relative positions of the probe sequence used for the construction of the Affymetrix HG_U95A-E or HG-U133A-B DNA-chips. In addition RNA and DNA specific primer/probe sequences were used to enable RNA and DNA specific measurements, by locating primer/probe sequences across Exon/Exon boundaries or within intron sequences respectively. All primer pairs were checked for specificity by conventional PCR reactions. To standardize the amount of sample RNA, GAPDH and RPL37A were selected as reference genes, since it was not differentially regulated in the samples analyzed. However, for most of the subsequent calculations the RPL37A gene expression was used for normalization. TaqMan validation experiments were performed showing that the efficiencies of the target and the control amplifications are approximately equal which is a prerequisite for the relative quantification of gene expression by the comparative ΔΔC_{T} method, known to those with skills in the art. As well as the technology provided by Perkin Elmer one may use other technique implementations like Lightcycler™ from Roche Inc. or iCycler from Stratagene Inc.

RNA was isolated from paraffin-embedded, formalin-fixed tissues (=FFPE tissues). Those skilled in the art are able to perform RNA extraction procedures. For example, total RNA from a 5 to 10 μm curl of FFPE tumor tissue can be extracted using the High Pure RNA Paraffin Kit (Roche, Basel, Switzerland), quantified by the Ribogreen RNA Quantitation Assay (Molecular Probes, Eugene, Oreg.) and qualified by real-time fluorescence RT-PCR of a fragment of RPL37A. In general 0.5 to 2 ng RNA of each qualified RNA extraction was assayed by qRT-PCR as described below. For a detailed analysis of gene expression by quantitative PCR methods, one will utilize primers flanking the genomic region of interest and a fluorescent labeled probe hybridizing in-between. Using the PRISM 7700 or 7900 Sequence Detection System of PE Applied Biosystems (Perkin Elmer, Foster City, Calif., USA) with the technique of a fluorogenic probe, consisting of an oligonucleotide labeled with both a fluorescent reporter dye and a quencher dye, one can perform such a expression measurement. Amplification of the probe-specific product causes cleavage of the probe, generating an increase in reporter fluorescence. Primers and probes were selected using the Primer Express software and localized mostly across exon/intron borders and large intervening non-transcribed sequences (>800 bp) to guarantee RNA-specificity or with in the 3′ region of the coding sequence or in the 3′ untranslated region. Primer design and selection of an appropriate target region is well known to those with skills in the art. Predefined primer and probes for the genes listed in Tables 1a and 1b can also be obtained from suppliers e.g. PE Applied Biosystems. All primer pairs were checked for specificity by conventional PCR reactions and gel electrophoresis. To standardize the RNA amount of sample GAPDH and RPL37A were selected as references, since they were not differentially regulated in the samples analyzed. To perform such an expression analysis of genes within a biological samples the respective primer/probes are prepared by mixing 25 μl of the 100 μM stock solution “Upper Primer”, 25 μl of the 100 μM stock solution “Lower Primer” with 12.5 μl of the 100 μM stock solution TaqMan-probe (FAM/Tamra) and adjusted to 500 μl with aqua dest (Primer/probe-mix). For each reaction 1.25 μl cDNA of the patient samples were mixed with 8.75 μl nuclease-free water and added to one well of a 96 Well-Optical Reaction Plate (Applied Biosystems Part No. 4306737). 1.5 μl of the Primer/Probe-mix described above, 12.5 μl Taq Man Universal-PCR-mix (2×) (Applied Biosystems Part No. 4318157) and 1 μl Water are then added. The 96 well plates are closed with 8 Caps/Strips (Applied Biosystems Part Number 4323032) and centrifuged for 3 minutes. Measurements of the PCR reaction are done according to the instructions of the manufacturer with a TaqMan 7700 from Applied Biosystems o. 20114) under appropriate conditions (2 min. 50° C., 10 min. 95° C., 0.15 min. 95° C., 1 min. 60° C.; 40 cycles). Prior to the measurement of so far unclassified biological samples control experiments will e.g. cell lines, healthy control samples, samples of defined therapy response could be used for standardization of the experimental conditions.

TaqMan validation experiments were performed showing that the efficiencies of the target and the control amplifications are approximately equal which is a prerequisite for the relative quantification of gene expression by the comparative AACT method, known to those with skills in the art. Herefor the SoftwareSDS 2.0 from Applied Biosystems can be used according to the respective instructions. CT-values are then further analyzed with appropriate software (Microsoft Excel™) or statistical software packages (e.g. SAS, GraphPad Prism4, Genedata Expressionist™). As well as the technology described above, provided by Perkin Elmer, one may use other technique implementations like Lightcycler™ from Roche Inc. or iCycler from Stratagene Inc. capable of real time detection of an RT-PCR reaction.

Of the first 51 TECHNO patients messenger ribonucleic acids of ER and genes of the 17q12, 8q24 and 11q13 ARCHEONs including Her-2/neu, c-Myc and CCND1 respectively, were isolated by an experimental method based on magnetic beads from Bayer HealthCare Diagnostics. In short, the FFPE slide is deparraffinized in xylol and ethanol, the pellet is washed with ethanol and dried at 55° C. for 10 minutes. The pellet is then lysed and proteinized overnight at 55° C. with shaking. After adding a binding buffer and the magnetic particles (Bayer HealthCare Diagnostics Research, Leverkusen, Germany) nucleic acids are bound to the particles within 15 minutes at room temperature. On a magnetic stand the supernatant is taken away and beads can be washed several times with washing buffer. After adding elution buffer and incubating for 10 min at 70° C. the supernatant is taken away on a magnetic stand without touching the beads. After normal DNAseI treatment for 30 min at 37° C. and inactivation of DNAse I the solution is used for reverse transcription-polymerase chain reaction (RT-PCR). The quality and quantity of RNA is checked by measuring absorbance at 260 nm and 280 nm. Pure RNA has an A260/A280 ratio of 1.9-2.0. Transcriptional activity of the genes was assessed with quantitative Reverse Transcriptase Taqman™ polymerase chain reaction (RT-PCR) analysis. We applied 40 cycles of nucleic acid amplification and used GAPDH and/or RPL37A as housekeeping genes at a cycle threshold (CT) of 28. We calculated a normalized 40-normalized tagte gene CT value score or 2^{(40-normalized target gene CT value)} relative gene copy numbers that correlates proportionally to RNA transcription levels. By designing DNA and RNA specific Primer/Probes for target genes, it was possible to omit the DNAse treatment, resulting in higher amounts of nucleic acids for both RNA and DNA. Moreover by using differently fluorescent labels it was possible to detect DNA and RNA of a candidate gene within the very same reaction together with an internal spike control (consisting of the forward and reverse sequences linked to an artificial, non human nucleic acid sequence) thereby enabling a very robust, highly sensitive detection of candidate genes by using less amount of sample.

Fisher's exact test was used to investigate associations between candidate gene mRNA levels and established patient and tumor characteristics as well as with mRNA expression of other genes.

Because gene expression was used as a continuous variable Student's t-test (for normally distributed data), Mann-Whitney U test (for non-normally distributed data) and analysis of variance (ANOVA) were used to compare gene expression levels between different patient groups. When possible Overall survival (OS) and disease-free survival (DFS) were calculated from time of diagnosis to death or last follow-up and to malignant relapse, death without relapse or last follow-up, respectively. Survival curves and comparison by candidate gene transcriptional status were calculated with the Kaplan-Meier product-limit method and the Logrank test. The possible the Cox proportional hazards model and Wald X² test were used to assess the prognostic significance of various parameters for OS, DFS. All p-values are double-sided and observed differences are considered statistically significant when p<0.05.

### Results

Overall there was a good correlation between the different method IHC, FISH and qPCR methods, although the tumor cell content of the tissues varied substantially with 46% having a tumor cell content of >50% and 16% of the tumors having less than 20% tumor cells (median 40%). To approach dilutional problems resulting from low tumor cell content and to increase sensitivity and specificity of the qPCR methodology, we analyzed multiple neighbouring genes when looking for genomic alterations. Indeed it turned out, that the DNA alterations are best identified by PCR methodology by simultaneously detecting multiple genes of each ARCHEON (e.g. FLJ2091, TEM7, CACNB 1, PPARBP, CrkRS, NEUROD2, MLN64, MGC9753, Her-2/neu, GRB7, PSMD3, MLN51, NR1D1, THRA, WIRE, CDC6, RARA and TOP2A for 17q12 ARCHEON with MMP28 as reference gene; ZHX2, ZHX1, DERL1, ATAD2, ANXA13, RNF139, FBX032, MTSS1, TRIB1, NSE2, c-Myc, MLZE, FAM49B, DDEF1, ADCY8, KIAA0143, WISP1, TG, SLA, NDRG1 for the 8q24 ARCHEON with FLD207720 as 8q24 reference; MYEOV, CCND1, ORAOV1, FGF19, FGF4, FGF3, TMEM16A, FADD and PPFIA1 for the 11q13 ARCHEON with HTATIP as 11q13 reference gene) and is superior to single gene detection of Her-2/neu with regard to sensitivity and assay robustness. By combining the results of tumor cell content, Her-2/neu RNA expression and Her-2/neu amplification status, as depicted by qPCR, we obtained superior prognostic and predictive information the genomic status compared to conventional IHC/FISH testing in chemotherapy treated tumors+/−trastuzumab.

TABLE 1a
Genes differentially expressed and capable of predicting therapeutic success

| Gene Symbol | Gene No. | Chr. | Gene Description and Gene Function | Ref. Seq. | Protein Seq | Unigene I | OMIM |
|---|---|---|---|---|---|---|---|
| ZHX2 | 1 | 8q24 | homeobox gene, transcripiton factor, ALPHA- | NM_014943 | NP_055758 | Hs.377090 | 609185 |
| | | | FETOPROTEIN REGULATOR | | | | |
| ZHX1 | 2 | 8q24 | homeobox transcripiton factor, NF-Y Interaction | NM_001017926 | NP_001017926 | Hs.612084 | 604764 |
| | | | partner, CCAT Box regulatio [symbol] \ | | | | |
| DERL1 | 3 | 8q24 | retrotranslocation channel in ER, chaperone | NM_024295.3 | NP_077271 | Hs.241576 | 608813 |
| | | | function | | | | |
| ATAD2 | 4 | 8q24 | ATPase, chaperone-like function | NM_014109.2 | NP_054828 | Hs.370834 | |
| ANXA13 | 5 | 8q24 | Annexin family member involved in cell signaling | NM_004306.2 | NP_001003954 | Hs.181107 | 602573 |
| | | | and apoptosis | | | | |
| RNF139 | 6 | 8q24 | Ring finger protein, ER function, transmembrane | NM_007218.3 | NP_009149 | Hs.632057 | 603046 |
| | | | ubiquitine ligase | | | | |
| FBX032 | 7 | 8q24 | F-box protein family, ubiquitine ligase function | NM_148177.1 | NP_478136 | Hs.403933 | 606604 |
| MTSS1 | 8 | 8q24 | cytoskeletal organization, actin binfding function | NM_014751.2 | NP_055566 | Hs.336994 | 608486 |
| TRIB1 | 9 | 8q24 | phosphoprotein controlling MAPK activation, | NM_025195.2 | NP_000688.1 | Hs.444947 | 609461 |
| | | | NFkB signaling, apoptosis regu [symbol] \ | | | | |
| NSE2 | 10 | 8q24 | membrane protein with potetial cell-cell | NM_174911.3 | NP_777571 | Hs.124951 | 609483 |
| | | | adhesion function due to interaction [symbol] \ | | | | |
| c-MYC | 11 | 8q24 | multifunctional, nuclear phosphoprotein | NM_002467.3 | NP_002458 | Hs.202453 | 190080 |
| | | | involved in proliferation, apoptosis ar [symbol] \ | | | | |
| MLZE | 12 | 8q24 | leucine zipper protein in volved in | NM_031415.2 | NP_113603 | Hs.133244 | 608384 |
| | | | protein interactions, | | | | |
| PVT1 | 13 | 8q24 | protein of so far unknown function | NM_001037234.1 | P07355 | Hs.133107 | 165140 |
| FAM49B | 14 | 8q24 | protein of so far unknown function | NM_016623.3 | NP_057707 | Hs.126941 | |
| DDEF1 | 15 | 8q24 | GTPase-activating protein, function fro | NM_018482.2 | NP_060952 | Hs.106015 | 605953 |
| | | | cell motility and cell spreading | | | | |
| ADCY8 | 16 | 8q24 | adenylate cyclase, cAMP signalling, Raf | NM_001115.1 | NP_001106 | Hs.591859 | 103070 |
| | | | and PKC regulated | | | | |
| KIAA0143 | 17 | 8q24 | protein of so far unknown function | NM_015137.1 | NP_055952 | Hs.204564 | |
| HHLA1 | 18 | 8q24 | HERV-H LTR-associating 1 | NM_005712.1 | NP_005703.1 | Hs.285026 | 604109 |
| KCNQ3 | 19 | 8q24 | potassium voltage-gated channel, KQT-like | NM_015137.1 | NP_004510 | Hs.374023 | 602232 |
| | | | subfamily, member 3; neuronal f [symbol] \ | | | | |
| LRRC6 | 20 | 8q24 | leucine rich repeat containing 6 | NM_012472 | NP_036604 | Hs.591865 | |
| TMEM71 | 21 | 8q24 | transmembrane protein; function in | NM_144649 | NP_653250 | Hs.293842 | |
| | | | prostaglandin signalling derived from CO [symbol] \ | | | | |
| PHF20L1 | 22 | 8q24 | tudor domain protein, RNA binding | NM_016018 | NP_057102 | Hs.304362 | |
| TG | 23 | 8q24 | thyroid hormone precursor | NM_003235 | NP_003226 | Hs.584811 | 188450 |
| SLA | 24 | 8q24 | Src-like-adaptor | NM_006748 | NP_006739 | Hs.75367 | 601099 |
| WISP1 | 25 | 8q24 | WNT1 inducible signaling pathway protein 1 | NM_003882 | NP_003873 | Hs.492974 | 603398 |
| NDRG1 | 26 | 8q24 | N-myc downstream regulated gene 1; alpha/beta | NM_006096 | NP_006087 | Hs.372914 | 605262 |
| | | | hydrolase superfamily | | | | |
| ST3GAL1 | 27 | 8q24 | sialyltransferase, thyroid function | NM_003033 | NP_003024 | Hs.584803 | 607187 |
| MYEOV | 28 | 11q13 | myeloma overexpressed gene, epigenetic | NM_138768 | NP_620123 | Hs.523848 | 605625 |
| | | | inactivation is associated with es [symbol] \ | | | | |
| CCND1 | 29 | 11q13 | cell cycle regulation, regulates CDK4/6 at G1/S | NM_053056.2 | NP_444284 | Hs.523852 | 168461 |
| | | | transition, interaction with Rt [symbol] \ | | | | |
| FLJ42258 | 30 | 11q13 | transcriptional coactivator for hormone function, | NM_001004327 | NP_001004327 | Hs.632135 | |
| | | | cell cycle regulation and cel [symbol] \ | | | | |
| ORAOV1 | 31 | 11q13 | oral cancer overexpressed 1 | NM_153451 | NP_703152 | Hs.523854 | 607224 |
| FGF19 | 32 | 11q13 | fibroblast growth factor, cell cycle regulation, | NM_005117 | NP_005108 | Hs.249200 | 603891 |
| | | | high affinity to FGFR4, WNT si [symbol] \ | | | | |
| FGF4 | 33 | 11q13 | fibroblast growth factor; cell cycle regulation, | NM_002007 | NP_001998 | Hs.1755 | 164980 |
| | | | interaction with SHH, TGFB an [symbol] \ | | | | |
| FGF3 | 34 | 11q13 | fibroblast growth factor; cell cycle regulation, | NM_005247 | NP_005238 | Hs.37092 | 164950 |
| | | | MMTV integration site int2, | | | | |
| TMEM16A | 35 | 11q13 | transmembrane protein; oral cancer overexpressed | NM_018043 | NP_060513 | Hs.503074 | 610108 |
| | | | 2; tumor amplified; d [symbol] \ | | | | |
| FADD | 36 | 11q13 | adaptor molecule that interacts with cell surface | NM_003824 | NP_003815 | Hs.86131 | 602457 |
| | | | receptors (TNFR); mediates | | | | |
| PPFIA1 | 37 | 11q13 | LAR protein-tyrosine phosphatase-interacting | NM_003626 | NP_003617 | Hs.530749 | |
| | | | protein family member, focal [symbol] \ | | | | |
| CCTN | 38 | 11q13 | cell adhesion; overexpressed in cancer; tumor cell | NM_005231 | NP_005222 | Hs.632133 | 164765 |
| | | | invasion, degraded by ca [symbol] \ | | | | |
| SHANK2 | 39 | 11q13 | CCTN interacting protein | NM_012309 | NP_036441 | Hs.268726 | 603290 |

[symbol]

indicates data missing or illegible when filed

TABLE 1b
Genes differentially expressed and capable of predicting therapeutic success:

| Gene Symbol | Gene No. | Chr. | Gene Description and Gene Function | Ref. Seq. | Protein Seq | Unigene ID | OMIM |
|---|---|---|---|---|---|---|---|
| MLLT6 | 40 | 17q12 | yeloid/lymphoid or mixed-lineage leukemia | NM_005937 | NP_005928 | Hs.91531 | 600328 |
| | | | (trithorax homolog, D [symbol] \ | | | | |
| PCGF2 | 41 | 17q12 | polycomb group ring finger 2; maintain the | NM_007144 | NP_009075 | Hs.371617 | 600346 |
| | | | transcription repressic [symbol] \ | | | | |
| PSMB3 | 42 | 17q12 | proteasome (prosome, macropain) subunit, | NM_002795 | NP_002786 | Hs.82793 | 602176 |
| | | | multicatalytic protein [symbol] \ | | | | |
| PIP5K2B | 43 | 17q12 | phosphatidylinositol-4-phosphate 5-kinase, | NM_003559 | NP_003550 | Hs.260603 | 603261 |
| | | | protein interacts with [symbol] \ | | | | |
| CCDC49 | 44 | 17q12 | coiled-coil domain containing 49, protein | NM_017748 | NP_060218 | Hs.406223 | 7892 |
| | | | interaction | | | | |
| RPL23 | 45 | 17q12 | ribosomal protein L23, part of complex that | NM_000978 | NP_000969 | Hs.406300 | 603662 |
| | | | catalyzes ribosomal [symbol] \ | | | | |
| LASP1 | 46 | 17q12 | LIM and SH3 protein 1; LASP-1 has an | NM_006148 | NP_006139 | Hs.548018 | 602920 |
| | | | essential role in tumor c [symbol] \ | | | | |
| FBX047 | 47 | 17q12 | F-box protein 47: protein degradation | NM_001008777 | NP_001008777 | Hs.549536 | 609498 |
| PLXDC1 | 48 | 17q12 | plexin domain containing 1; transmembrane | NM_020405 | NP_065138 | Hs.125036 | 606826 |
| | | | domain protein; turr [symbol] \ | | | | |
| ARL5C | 49 | 17q12 | ADP-ribosylation factor-like 5C; smIall | XM_372668 | | Hs.568007 | |
| | | | GTPase; vesicle transpor [symbol] \ | | | | |
| CACNB1 | 50 | 17q12 | calcium channel, voltage-dependent, beta | NM_000723 | NP_000714 | Hs.635 | 114207 |
| | | | 1 subunit; modulating [symbol] \ | | | | |
| RPL19 | 51 | 17q12 | ribosomal protein L19; component of the 60S | NM_000981 | NP_000972 | Hs.381061 | 180466 |
| | | | subunit; protein synthesis | | | | |
| STAC2 | 52 | 17q12 | SH3 and cysteine rich domain 2; regulation | NM_198993 | NP_945344 | Hs.145068 | |
| | | | of enzymes by intra [symbol] \ | | | | |
| FBXL20 | 53 | 17q12 | F-box and leucine-rich repeat protein 20; | NM_032875 | NP_116264 | Hs.462946 | 609086 |
| | | | protein degradation; do [symbol] \ | | | | |
| PPARBP | 54 | 17q12 | PPAR binding protein; cofactor required | NM_004774 | NP_004765 | Hs.462956 | 604311 |
| | | | for SP1 activation) corr [symbol] \ | | | | |
| CRKRS | 55 | 17q12 | Cdc2-related kinase; cell cycle regulation | NM_016507 | NP_057591 | Hs.416108 | |
| NEUROD2 | 56 | 17q12 | neurogenic differentiation 2; neurogenic basic | NM_006160 | NP_006151 | Hs.322431 | 601725 |
| | | | helix-loop-helix (bl [symbol] \ | | | | |
| PPP1R1B | 57 | 17q12 | protein phosphatase 1, regulatory (inhibitor) | NM_032192 | NP_115568 | Hs.286192 | 604399 |
| | | | subunit 1B; dopami [symbol] \ | | | | |
| STARD3 | 58 | 17q12 | START domain containing 3; Cholesterol | NM_006804 | NP_006795 | Hs.77628 | 607048 |
| | | | homeostasis; | | | | |
| TCAP | 59 | 17q12 | titin-cap (telethonin); substrate of titin kinase, | NM_003673 | NP_003664 | Hs.514146 | 604488 |
| | | | critical to sarcome [symbol] \ | | | | |
| PNMT | 60 | 17q12 | phenylethanolamine N-methyltransferase; | NM_002686 | NP_002677 | Hs.1892 | 171190 |
| | | | Tyrosine metabolism; [symbol] \ | | | | |
| PERLD1 | 61 | 17q12 | per1-like domain containing 1; | NM_033419 | NP_219487 | Hs.462971 | |
| Her-2/neu | 62 | 17q12 | v-erb-b2 erythroblastic leukemia viral oncogene | NM_004448 | NP_004439 | Hs.446352 | 164870 |
| | | | homolog 2; rece [symbol] \ | | | | |
| C17orf37 | 63 | 17q12 | chromosome 17 open reading frame 37; | NM_032339 | NP_115715 | Hs.333526 | |
| | | | chaperone function,; pc [symbol] \ | | | | |
| GRB7 | 64 | 17q12 | growth factor receptor-bound protein 7; | NM_001030002 | NP_001025173 | Hs.86859 | 601522 |
| | | | intracellular signaling co [symbol] \ | | | | |
| ZNFN1A3 | 65 | 17q12 | zinc finger protein, subfamily 1A, 3 (Aiolos); | NM_012481 | NP_036613 | Hs.444388 | 606221 |
| | | | chromatin remodeli [symbol] \ | | | | |
| ZPBP2 | 66 | 17q12 | secreted protein; ZPBP2 mRNA was | NM_198844 | NP_942141 | Hs.367245 | 608499 |
| | | | coexpressed with ZPBP ml [symbol] \ | | | | |
| GSMDL | 67 | 17q12 | Gasdermin like Protein; LTR element of | NM_001042471 | NP_001035936 | | Hs.306777 |
| | | | HERV-H with reverse or [symbol] \ | | | | |
| ORMDL3 | 68 | 17q12 | ORM1-like 3; putative ER function | NM_139280 | NP_644809 | | Hs.514151 |
| GSMD1 | 69 | 17q12 | gasdermin 1; differentiation of hair follicle | NM_178171 | NP_835465 | | Hs.448873 |
| | | | cells; suppreessed in gastric cancer cells | | | | |
| PSMD3 | 70 | 17q12 | proteasome (prosome, macropain) 26S subunit, | NM_002809 | NP_002800 | | Hs.12970 |
| | | | non-ATPase, 3; [symbol] \ | | | | |
| CSF3 | 71 | 17q12 | colony stimulating factor 3; strong immune | NM_000759 | NP_000750 | 138970 | Hs.2233 |
| | | | regulator of T cells; c [symbol] \ | | | | |
| THRAP4 | 72 | 17q12 | thyroid hormone receptor associated protein 4; | NM_014815 | NP_055630 | 607000 | Hs.462983 |
| | | | interaction with E [symbol] \ | | | | |
| THRA | 73 | 17q12 | thyroid hormone receptor, alpha (erythroblastic | NM_003250 | NP_003241 | 190120 | Hs.724 |
| | | | leukemia viral (v- [symbol] \ | | | | |
| NR1D1 | 74 | 17q12 | nuclear receptor subfamily 1, group D, | NM_021724 | NP_068370 | 602408 | Hs.592130 |
| | | | member 1; Overexpressi [symbol] \ | | | | |
| CASC3 | 75 | 17q12 | cancer susceptibility candidate 3; metastatic | NM_007359 | NP_031385 | 606504 | Hs.592129 |
| | | | lymph node 51 “MI [symbol] \ | | | | |
| RAPGEFL1 | 76 | 17q12 | Rap guanine nucleotide exchange factor | NM_016339 | NP_057423 | | Hs.632254 |
| | | | (GEF)-like 1; RAS activ [symbol] \ | | | | |
| WIRE | 77 | 17q12 | WASP interacting protein (WIP)-related | NM_133264 | NP_573571 | 609692 | Hs.421622 |
| | | | protein; potential link be [symbol] \ | | | | |
| CDC6 | 78 | 17q12 | CDC6 cell division cycle 6: initiation of | NM_001254 | NP_001245 | 602627 | Hs.405958 |
| | | | DNA replication; interacti [symbol] \ | | | | |
| RARA | 79 | 17q12 | retinoic acid receptor, alpha; regulated by | NM_000964 | NP_000955 | 180240 | Hs.137731 |
| | | | ER; Retinoid-induced [symbol] \ | | | | |
| GJC1 | 80 | 17q12 | gap junction protein, chi 1, 31.9 kDa | NM_152219 | NP_689343 | 607425 | Hs.444663 |
| | | | (connexin 31.9); heart funct [symbol] \ | | | | |
| TOP2A | 81 | 17q12 | topoisomerase (DNA) II alpha 170 kDa; | NM_001067 | NP_001058 | 126430 | Hs.156346 |
| | | | controls and alters the to [symbol] \ | | | | |
| IGFBP4 | 82 | 17q12 | insulin-like growth factor binding | NM_001552 | NP_001543 | 146733 | Hs.462998 |
| | | | protein 4; binds both insulin-like [symbol] \ | | | | |
| TNS4 | 83 | 17q12 | tensin 4; ist prostate restricted | NM_032865 | NP_116254 | 608385 | Hs.438292 |
| | | | expression is down-regulated in p [symbol] \ | | | | |
| CCR7 | 84 | 17q12 | chemokine (C-C motif) receptor 7; member of | NM_001838 | NP_001829 | 600242 | Hs.370036 |
| | | | the G protein-cou [symbol] \ | | | | |
| SMARCE1 | 85 | 17q12 | SWI/SNF related, matrix associated, actin | NM_003079 | NP_003070 | 603111 | Hs.547509 |
| | | | dependent regulator [symbol] \ | | | | |

[symbol]

indicates data missing or illegible when filed

### ZHX2

### Other Aliases: KIAA0854

By sequencing clones obtained from a size-fractionated brain cDNA library, ZHX2 has been cloned. The deduced 837-amino acid protein shares about 40% identity with mouse ZHX1. RT-PCR ELISA detected expression in all tissues examined, with highest levels in ovary, followed by lung, heart, kidney, brain, and liver. Intermediate expression was detected in pancreas, spleen, testis, and skeletal muscle. The deduced human protein has a calculated molecular mass of 92 kD. It contains 2 C2H2-type zinc finger motifs and 5 homeodomains (HD), with a unique proline-rich region between HD1 and HD2. Northern blot analysis detected a 4.4-kb transcript expressed at variable levels in all tissues examined. Kawata et al. (2003) cloned mouse Zhx2. The mouse and human ZHX2 proteins share 87% amino acid identity. Northern blot analysis detected Zhx2 expression in all mouse tissues examined.

By yeast 2-hybrid analysis and in vitro pull-down assays, a direct interaction between ZHX1 and ZHX2 has been demonstrated. ZHX2 could also form homodimers in vivo and in vitro. Both interactions required an extensive region around HD1. ZHX2 also interacted with the activation domain of NYFA (189903), and this interaction required the HD1 and HD2 region of ZHX2. Immunoprecipitation analysis detected an endogenous interaction between ZHX2 and NYFA in human embryonic kidney cells. Furthermore, ZHX2 was able to repress reporter activity driven by a CDC25C (157680) promoter, which contains 3 NFY-binding sequences.

### ZHX1

NFYA, NFYB, and NFYC comprise the heterotrimeric transcription factor known as nuclear factor Y (NF-Y), or CCAAT-binding protein (CBF). NF-Y binds many CCAAT box elements and Y box elements, which are inverted CCAAT boxes. Mutations of these elements that disrupt the binding of NF-Y result in decreased transcription from various tissue-specific and inducible promoters. To identify proteins that interact with NF-Y and that may play a role in tissue-specific or hormone-inducible promoter activity, a human liver cDNA library using a yeast 2-hybrid system with the NFYA subunit as bait has been screened. A partial ZHX1 cDNA lacking 5-prime coding sequence has been identified and the remaining ZHX1 coding sequence has been cloned. The predicted 873-amino acid ZHX1 protein contains 2 N-terminal zinc fingers, 5 central and C-terminal homeodomains, a C-terminal acidic region, and 2 putative nuclear localization signals. Human and mouse ZHX1 share 91% amino acid sequence identity. ZHX1 specifically interacts with NFYA both in vivo and in vitro. This interaction does not require the zinc fingers of ZHX1. Northern blot analysis detected major 4.5- and 5-kb ZHX1 transcripts in all tissues tested, namely heart, lung, liver, pancreas, kidney, brain, skeletal muscle, and placenta. The 5-kb transcript was more highly expressed than the 4.5-kb transcript in most of these tissues.

### DERL1

### Other Aliases: DER-1, DER1, FLJ13784, FLJ42092, MGC3067, PRO2577

Derlin-1 is part of a retrotranslocation channel that is associated with both the polyubiquitination and p97-ATPase machineries at the endoplasmic reticulum membrane. Derlin-1 interacts with the N-terminal domain of PNGase via its cytosolic C-terminus. PNGase distributed in two populations; ER-associated and free in the cytosol, which suggests the deglycosylation process can proceed at either site. Derlin-1 interacts with US11, a virally encoded ER protein that specifically targets MHC class I heavy chains for export from the ER, as well as with VIMP, a novel membrane protein that recruits the p97 ATPase and its cofactor. Derlin-1 is an important factor for the extraction of certain aberrantly folded proteins from the mammalian ER.

### ATAD2

### Other Aliases: DKFZp667N1320, MGC131938, MGC29843, MGC5254, PRO2000

ATAD2 is a member of a large family of ATPases, whose key feature is that they share a conserved region of about 220 amino acids that contains an ATP-binding site. The proteins that belong to this family either contain one or two AAA (ATPases Associated with diverse cellular Activities) domains. AAA family proteins often perform chaperone-like functions that assist in the assembly, operation, or disassembly of protein complexes. The protein encoded by this gene contains two AAA domains, as well as a bromodomain.

### ANXA13

### Other Aliases: ANX13, ISA

ANXA13 encodes a member of the annexin family. Members of this calcium-dependent phospholipid-binding protein family play a role in the regulation of cellular growth and in signal transduction pathways. The specific function of this gene has not yet been determined; however, it is associated with the plasma membrane of undifferentiated, proliferating endothelial cells and differentiated villus enterocytes. Alternatively spliced transcript variants encoding different isoforms have been identified.

### RNF139

### Other Aliases: HRCA1, MGC31961, RCA1, TRC8

The protein encoded by this gene is a multi-membrane spanning protein containing a RING-H2 finger. This protein is located in the endoplasmic reticulum, and has been shown to possess ubiquitin ligase activity. This gene was found to be interrupted by a t(3:8) translocation in a family with hereditary renal and non-medulary thyroid cancer. Studies of the Drosophila counterpart suggested that this protein may interact with tumor suppressor protein VHL, as well as with COPS5/JAB1, a protein responsible for the degradation of tumor suppressor CDKN1B/P27KIP. FBXO32

### Other Aliases: ATROGIN1, FLJ32424, Fbx32, MAFbx, MGC33610

This gene encodes a member of the F-box protein family which is characterized by an approximately 40 amino acid motif, the F-box. The F-box proteins constitute one of the four subunits of the ubiquitin protein ligase complex called SCFs (SKP1-cullin-F-box), which function in phosphorylation-dependent ubiquitination. The F-box proteins are divided into 3 classes: Fbws containing WD-40 domains, Fbls containing leucine-rich repeats, and Fbxs containing either different protein-protein interaction modules or no recognizable motifs. The protein encoded by this gene belongs to the Fbxs class and contains an F-box domain. This protein is highly expressed during muscle atrophy, whereas mice deficient in this gene were found to be resistant to atrophy. This protein is thus a potential drug target for the treatment of muscle atrophy. Alternative splicing of this gene results in two transcript variants encoding two isoforms of different sizes.

### MTSS1

### Other Aliases: FLJ44694, KIAA0429, MIM, MIMA, MIMB

MTSS1 is called “Metastasis Suppressor 1” or “Missing In Metastasis”. However MTSS1 is unlikely to be a metastasis suppressor but acts as a scaffold protein that interacts with Rac, actin and actin-associated proteins to modulate lamellipodia formation. Data indicate that down-regulation of MTSS1 expression can occur in bladder cancer cell lines but is not associated with increased invasive behaviour. MTSS1 protein and insulin receptor tyrosine kinase substrate p53 have a conserved novel actin bundling/filopodium-forming domain. It may be involved in cytoskeletal organization. C-terminal half of mouse MTSS1 protein, which contains the WH2 domain, binds actin monomers. Steady state and kinetic assembly assays showed that MTSS1 inhibits pointed-end actin assembly and actin monomer nucleotide exchange. Overexpression of MTSS1 in NIH 3T3 cells caused formation of abnormal actin structures. MTSS1 transcripts in the outer root sheath of anagen hair follicles, but not in the interfollicular epithelium. MTSS1 RNA and protein also accumulated at sites of inappropriately active sonic hedgehog signaling, such as tumor epithelium of human basal cell carcinomas.

### TRIB1

### Other Aliases: C8FW, GIG2, SKIP1

Tribbles homolog, that controls both the extent and the specificity of MAPK kinase activation of MAPK. By screening a thyroid cDNA library with dog Trib2, human TRIB1 has been cloned and named C8FW. Human TRIB1 and dog Trib2 share about 70% amino acid identity. Based on its sequence similarity with TRIB3, TRIB1 has been identified independently and named SKIP 1. The deduced 372-amino acid TRIB1 protein contains a serine/threonine kinase-like domain. Moreover, using a transcription expression screen for genes regulating the IL8 promoter in HeLa cells, TRIB1 has been identified. The deduced protein is likely to be inactive, since it lacks the active-site lysine within the serine/threonine kinase-like domain. Quantitative real-time PCR of several tissues detected highest TRIB1 expression in skeletal muscle, thyroid, pancreas, peripheral blood leukocytes, and bone marrow. However, it was found that overexpression of TRIB1 in HeLa cells repressed the basal activity of the IL8 promoter by inhibiting AP1 activity. Overexpression of TRIB1 inhibited oncogenic Ras-driven AP1 activation and MEKK1-mediated API activation. ERK activation was enhanced by TRIB1. Communoprecipitation and yeast 2-hybrid assays showed that MEK1 interacted with both TRIB1 and TRIB3, and MKK4 interacted specifically with TRIB1. Cotransfection of MKK4 enhanced the level of TRIB1, indicating that the TRIB-MAPKK interaction stabilized TRIB1. The expression status of C-MYC, TRIB1 (alias C8FW), and FAM84B (alias NSE2) in the regions of 8q24 has been analyzed in esophageal carcinomas with distinct amplification of 8q24 by reverse transcriptase-polymerase chain reaction or immunohistochemical analysis (or both). However, no expression of TRIB1 was detected in esophageal squamous cell carcinomas, suggesting that C-MYC and TRIB1 may not be the amplification target of 8q24 in esophageal cancer. The genomic organization of 8q24 has been investigated in32 AML and two MDS cases with MYC-containing dmin. The minimally amplified region was shown to be 4.26 Mb in size, harboring five known genes, with the proximal and the distal amplicon breakpoints clustering in two regions of approximately 500 and 600 kb, respectively. Interestingly, in 23 (68%) of the studied cases, the amplified region was deleted in one of the chromosome 8 homologs at 8q24, suggesting excision of a DNA segment from the original chromosomal location according to the ‘episome model’. In one case, sequencing of both the dmin and del(8q) junctions was achieved and provided definitive evidence in favor of the episome model for the formation of dmin. Expression status of the TRIB1 and MYC genes, encompassed by the minimally amplified region, was assessed by northern blot analysis. The TRIB1 gene was found over-expressed in only a subset of the AML/MDS cases, whereas MYC, contrary to expectations, was always silent. The present study, therefore, strongly suggests that MYC is not the target gene of the 8q24 amplifications.

The transcription factor NF-κB plays important roles in inflammation and cell survival. Interestingly, NF-κB is critically involved in regulation of cell death and survival through transcriptional activation of genes important for apoptosis and cell proliferation, such as Casper/c-FLIP, c-IAPs, TRAF1, TRAF2, Bfl-1/A1, Bcl-Xl, Fas ligand, c-myc and cyclin D1. In a yeast two-hybrid screening for TNF ligand-associated molecules, SINK has been identified as an NF-κB-inducible protein sharing sequence homology to serine/threonine protein kinases. Overexpression of SINK inhibited NF-κB-dependent transcription induced by tumor necrosis factor (TNF) stimulation or its downstream signaling proteins but did not inhibit NF-κB translocation to the nucleus and binding to DNA. Co-immunoprecipitation and in vitro kinase assays indicated that SINK specifically interacted with the NF-κB transactivator p65 and inhibited p65 phosphorylation by the catalytic subunit of protein kinase A, which has previously been shown to regulate NF-κNB activation. Consistent with its role in inhibition of NF-κB-dependent transcription, SINK also sensitized cells to apoptosis induced by TNF and TRAIL (TNF-related apoptosis-inducing ligand). Taken together, these data suggest that SINK is critically involved in a novel negative feedback control pathway of NF-κB-induced gene expression. Importantly, SINK is identical to TRIB1.

### NSE2

Other Aliases: FLJ32440, MMS21, C8orf36

Using a proteomics approach to identify genes upregulated in breast cancer cell membranes, followed by database analysis and PCR of a pooled testis, fetal lung, and B-cell cDNA library, a gene named BCMP101 (“Breast Cancer Membrane Protein”) has been cloned, which is identical to NSE2. The deduced protein contains 310 amino acids. RT-PCR and immunohistochemical analyses demonstrated low BCMP101 expression in multiple normal tissues. However, high levels of BCMP10 mRNA were detected in breast carcinoma cells, with expression upregulated more than 2-fold in 6 of 7 breast carcinomas tested compared with adjacent normal tissue. Fluorescence-tagged BCMP101 showed widespread intracellular localization and significant expression on the plasma membrane, particularly in areas of cell-cell contact. In line with this an interaction of BCMP101 with alpha-1 catenin has been found in yeast two hybrid assays.

c-Myc

### Other Aliases: MYC

The protein encoded by this gene is a multifunctional, nuclear phosphoprotein that plays a role in cell cycle progression, apoptosis and cellular transformation. It functions as a transcription factor, that regulates transcription of specific target genes. Mutations, overexpression, rearrangement and translocation of this gene have been associated with a variety of solid tumors and leukemias/lymphomas including Burkitt lymphoma.

Interestingly, c-Myc gene is regulated by nitric oxide via inactivating NF-kappa B complex. Moreover, a role of c-Myc increasing susceptibility to tumor necrosis factor mediated apoptosis has been described.

c-Myc family genes affects oncogenesis through distinct sets of targets by transcriptional repression and activation. For example, c-Myc binds well to well conserved canonical E boxes resulting in a switch to glycolytic metabolism during cell proliferation or tumorigenesis. c-Myc has a pivotal function in the development of breast cancer. c-Myc amplification is an early event in breast cancer progression, while Her2/neu amplification may play a role in the later stage of tumor development. Gene amplification of c-Myc have been resumed to play a key role in regulating expression of its mRNA and protein in high-grade breast cancers. However, a marked intratumoral heterogeneity of c-Myc, CCND1 but not of c-erbB2 amplification in breast cancer has been observed. Data show that decreasing the c-Myc protein level in MCF-7 cells by RNAi could significantly inhibit tumor growth both in vitro and in vivo. Interestingly, c-Myc expression is regulated by ER alpha and 17-beta-estradiol has been shown to promote survival signals in breast cancer cells. Here, the c-Myc-dependent survival signal generated by E2 was dependent upon basal levels of mTOR (mammalian target of rapamycin) and two upstream regulators of mTOR, phosphatidylinositol 3-kinase and phospholipase D (PLD). c-Myc also antagonizes the induction of p21Cip1 mediated by oncogenic H-, K-, and N-Ras and by constitutively activated Raf and ERK2. Moreover, c-Myc downregulation and release from the endogenous p21WAF1/CIP1 promoter contributes to transcriptional activation of the p21WAF1/CIP1 in HeLa cells.

c-Myc expression shows a positive association with increasing grade of breast carcinoma. c-Myc has a role in tumor progression in BRCA1-associated breast cancers. c-Myc binds to the hTERT promoter and is involved in the pathway for regulation of cellular immortalization through BRCA1. A complex of Nmi and BRCA1 inhibits c-Myc-induced human telomerase reverse transcriptase gene promoter activity in breast cancer. The c-myc downstream pathway includes other chromosome 17q genes nm23-H1 and nm23-H2. Results also show that Ser727/Tyr701-phosphorylated Stat1 plays a key role as a prerequisite for the ATRA-induced down-regulation of c-Myc; cyclins A, B, D2, D3, and E; and simultaneous up-regulation of p27Kip1, associated with arrest in the G0/G1 phase. In addition, c-Myc promotes cell growth and cancer development partly by inhibiting the growth inhibitory functions of Smads by directly interacting with Smad2 and Smad3 involved in TGF-beta signaling.

p53 represses c-Myc transcription through a mechanism that involves histone deacetylation. Elevated levels of c-Myc counteract p53 activity in human tumor cells. Myc overexpression causes DNA damage in vivo and the ATM-dependent response to this damage is critical for p53 activation, apoptosis, and the suppression of tumor development. Overexpression of c-Myc disrupts the repair of double-strand DNA breaks, resulting in a several-magnitude increase in chromosomal breaks and translocations

Nuclear c-Myc interacts with Max, binds to the specific DNA sequence, and plays an important role in stimulation of normal intestinal epithelial cell proliferation. c-Myc together with its heterodimeric partner, Max, occupy >15% of gene promoters tested in Burkitt lymphoma cells. Dual roles for p300-CBP-associated factor have been observed for c-Myc regulation: as a c-Myc coactivator that stabilizes c-Myc and as an inducer of c-Myc instability via direct c-Myc acetylation. p300 can acetylate DNA-bound Myc:Max complexes. In turn acetylated Myc:Max heterodimers efficiently interact with Miz-1 Site-specific ubiquitination regulating the switch between an activating and a repressive state of the c-Myc protein. Overexpressed c-Myc plays a role in global transcriptional regulation in some cancer cells and functions in malignant transformation. c-Myc has been described as a critical substrate in the GSK3beta survival-signaling pathway mutations in beta-catenin correlate with c-myc overexpression.

Myc is an integral part of a novel HIF-1alpha pathway, which regulates a distinct group of Myc target genes in response to hypoxia. Myc stimulates VEGF production by a rapamycin- and LY294002-sensitive pathway. C-Myc overexpression was significantly associated with high sVEGF and normal sFlt-1 level in DLBCL patients, suggesting a complex interrelationship between c-Myc oncogene expression and angiogenic regulators. Repression of alpha-fetoprotein gene expression under hypoxic conditions in cancer cells has been shown and a negative hypoxia response element that mediates opposite effects of hypoxia inducible factor-1 and c-Myc has been characterized.

### DDEF1

Gene aliases: PAP; PAG2; AMAP1; ASAP1; ZG14P; KIAA1249

Results support a model that regulation of GAP (GTPase-activating protein) activity of ASAP1 involves conformational changes, coincident with recruitment to a membrane surface and following the specific binding of phosphatidylinositol 4,5-bisphosphate. DDEF-1 alters cell motility through the deactivation of ARF1. In contrast, the inhibition of cell spreading by DDEF-1 was not dependent on GAP activity, indicating that spreading and motility are altered by DDEF-1 through different pathways. POB1 interacts with DDEF1 through its proline-rich motif, thereby regulating cell migration. DDEF1 is involved in peripheral focal adhesions, directed by CRKL protein. DDEF1 overexpression may be a pathogenetically relevant consequence of chromosome 8q amplification, which commonly occurs in high-grade uveal melanomas.

### ADCY8

### Other Aliases: ADCY3, HBAC1

Adenylate cyclase 8 is a membrane bound enzyme that catalyses the formation of cyclic AMP from ATP. The enzymatic activity is under the control of several hormones, and different polypeptides participate in the transduction of the signal from the receptor to the catalytic moiety. Stimulatory or inhibitory receptors (Rs and Ri) interact with G proteins (Gs and Gi) that exhibit GTPase activity and they modulate the activity of the catalytic subunit of the adenylyl cyclase. A direct interaction between the N terminus of adenylyl cyclase ADCY8 and the catalytic subunit of protein phosphatase 2A was shown

### KIAA0143

### Other Aliases: DKFZp781J0562

### HHLA1

### Other Aliases: PLA2L

Human endogenous retroviruses (HERVs) are repetitive elements, derived from ancient germline retroviral infections, that have increased in copy number by further rounds of infection, retrotransposition, and/or duplication. The HERV-H family has been shown to play a role in the expression of a variety of adjacent genes. PLA2L (phospholipase A2-like) has been isolated as a teratocarcinoma cell line transcript, which initiates in the long terminal repeat (LTR) of an HERV-H element present in an intron and splices into downstream exons. They found that the teratocarcinoma cells contained additional, alternatively spliced PLA2L mRNAs, designated AF6 through -8, which lack the coding regions for the phospholipase A2 (PLA2)-like domains. PLA2L turned out to be a tripartite fusion transcript expressed from the HERV-H element's promoter and containing exons from a novel gene, HHLA1, and from OC90, a gene encoding an inner ear protein with PLA2 domains. The coding regions of the AF6, -7, and -8 mRNAs are derived only from the HHLA1 gene and encode a predicted 305-amino acid protein. HHLA1 and OC90 genes are normally expressed independently from different promoters. The intergenic splicing event that generates PLA2L is specific to teratocarcinoma cells. The HERV-H element is located within an intron of HHLA1 and the OC90 gene is located less than 10 kb downstream of HHLA1. The HERV-H element at this locus integrated 15 to 20 million years ago since it is present in chimpanzee and gorilla but absent in orangutan and lower primates.

### KCNQ3

### Other Aliases: BFNC2, EBN2, KV7.3

The M channel is a slowly activating and deactivating potassium channel that plays a critical role in the regulation of neuronal excitability. The M channel is formed by the association of the protein encoded by this gene and one of two related proteins encoded by the KCNQ2 and KCNQ5 genes, both integral membrane proteins. M channel currents are inhibited by M1 muscarinic acetylcholine receptors and activated by retigabine, a novel anti-convulsant drug. Defects in this gene are a cause of benign familial neonatal convulsions type 2 (BFNC2), also known as epilepsy, benign neonatal type 2 (EBN2). Src associates with KCNQ2-5 subunits but phosphorylates only KCNQ3-5.

### LRRC6

### Other Aliases: LRTP, TSLRP

LRRC6 is a leucine rich repeat protein involved in protein-protein interactions.

### TMEM71

### Other Aliases: FLJ33069, MGC111188

TMEM71 is a transmembrane protein bearing similarities to the prostaglandin E receptor. We conclude, that the that this gene may be involved in inflammatory and stress response processes and that its importance in tumor development function is associated with p53 and COX function.

### PHF20L1

### Other Aliases: CGI-72, MGC64923

PHF20L1 is a PHD finger protein that may be involved in transcription regulation.

### TG

### Other Aliases: AITD3

Thyroglobulin is the glycoprotein precursor to the thyroid hormones. Its synthesis under normal physiological conditions is restricted to the thyroid gland with its metabolism having seemingly wasteful features. It has a molecular weight of 660,000, with 2 identical subunits of MW 300,000 and 10% sugars; yet its complete hydrolysis yields only 2 to 4 molecules of the iodothyronines, T4 and T3. There is an increased prevalence of autoimmune thyroiditis in women with breast cancer as determined by anti-TG and anti TPO antibodies. The finding that 25.6% women with breast cancer had beyond doubt a thyroid disorder, though subclinical, and another 26.8% are candidates of thyroid disease with a positive antibodies supports the hypothesis of a relationship of certain types of thyroid disease and (some types) of breast cancer. The expression of TG is regulated by estrogens and affected by anti-hormonal treatment (e.g. Tamoxifen treatment). Patients with recurrent breast cancer having elevated TSH and lower levels of T3 and T4 have worse prognosis.

We have found, that thyroglobulin is also expressed breast tumors, in particular tumors with alterations at chromosome 8q24 (˜frequency of about 20% of all breast tumors). As this tumors aberrantly produce the hormone precursor and are highly immunogenic we can now answer, why breast cancer patients are predisposed to autoimmune thyroiditis and why especially these patients have a worse outcome. Moreover, we have found, that measurement of thyroid function parameters (such as determination of serum levels of TG, T3, T4, TSH, PRL and autoantibodies raised against TG and TPO in combination with sHer-2/neu and CRP) are useful to determine patients with genomic alterations of the 8q24 locus having benefit from Herceptin treatment. We have found, that particularly the serum levels of anti-TG autoantibodies in serum Her-2/neu positive serum samples

### SLA

### Other Aliases: SLA1, SLAP

SLA has been isolated using the 2-hybrid system to screen for molecules that interact with the cytoplasmic domain of Eck, a mouse receptor protein kinase. The predicted 281-amino acid protein has both SH3 and SH2 adaptor motifs similar to those in the Src family of nonreceptor tyrosine kinases but had no catalytic domain. Therefore the protein was named Slap (Src-like adaptor protein). Recombinant Slap was shown to bind to activated Eck receptor tyrosine kinase. By molecular cloning the SLA protein, has been demonstrated to be embedded within the genomic organization of the human thyroglobulin gene. The SLA gene was identified by exon trapping on overlapping cosmids encompassing the largest TG intron. A 2.6-kb transcript, with the highest levels of expression in fetal brain and lung, was detected on Northern blots. Two full-length cDNAs (1 alternatively spliced) were isolated from a fetal brain library, both containing an open reading frame of 276 amino acids but lacking a catalytic tyrosine kinase domain. The gene showed a high degree of cross-species similarity and appeared to be transcribed in the direction opposite to TG. SLA has also been symbolized SLAP (which has been used for sarcolemmal-associated protein). SLA is a negative regulator of T-cell receptor signaling. SLA and SLA2 are both involved in downregulating T and B cell-mediated responses.

### WISP1

### Other Aliases: CCN4, WISP1c, WISP1i, WISP1tc

WISP1 encodes a member of the WNT1 inducible signaling pathway (WISP) protein subfamily, which belongs to the connective tissue growth factor (CTGF) family. WNT1 is a member of a family of cysteine-rich, glycosylated signaling proteins that mediate diverse developmental processes. The CTGF family members are characterized by four conserved cysteine-rich domains: insulin-like growth factor-binding domain, von Willebrand factor type C module, thrombospondin domain and C-terminal cystine knot-like domain. This gene may be downstream in the WNT1 signaling pathway that is relevant to malignant transformation. It is expressed at a high level in fibroblast cells, and overexpressed in colon tumors. The encoded protein binds to decorin and biglycan, two members of a family of small leucine-rich proteoglycans present in the extracellular matrix of connective tissue, and possibly prevents the inhibitory activity of decorin and biglycan in tumor cell proliferation. It also attenuates p53-mediated apoptosis in response to DNA damage through activation of the Akt kinase. It is 83% identical to the mouse protein at the amino acid level. Alternative splicing of this gene generates 2 transcript variants. Overexpression of WISP1 downregulates motility and invasion of lung cancer cells through inhibition of Rac activation. Overexpression of WISP1 has also been associated with breast cancer.

### NDRG1

### Other Aliases: CAP43, CMT4D, DRG1, GC4, HMSNL, NDR1, NMSL, PROXY1, RIT42, RTP, TARG1, TDD5

NDRG1 is a member of the N-myc downregulated gene family which belongs to the alpha/beta hydrolase superfamily. The protein encoded by this gene is a cytoplasmic protein involved in stress responses, hormone responses, cell growth, and differentiation. Mutation in this gene has been reported to be causative for hereditary motor and sensory neuropathy-Lom. NDRG1 is necessary but not sufficient for p53-mediated caspase activation and apoptosis. It plays a role in the regulation of microtubule dynamics and the maintenance of euploidy. NDRG1 has been described as a Myc negative target in human neuroblastomas and other cell types with overexpressed N- or c-myc. NDRG1 overexpression in cancer cells involves a state of hypoxia characteristic of cancer cells where the Cap43 protein becomes a signature for this hypoxic state and is downregulated by von Hippel-Lindau tumor suppressor protein in renal cancer cells.

### ST3GAL1

### Other Aliases: Gal-NAc6S, MGC9183, SIAT4A, SIATFL, ST3GalA.1, ST3GalIA, ST30

ST3GAL1 is a type II membrane protein that catalyzes the transfer of sialic acid from CMP-sialic acid to galactose-containing substrates. The encoded protein is normally found in the Golgi Apparatus, but can be proteolytically processed to a soluble form. Correct glycosylation of the encoded protein may be critical to its sialyltransferase activity. This protein, which is a member of glycosyltransferase family 29, can use the same acceptor substrates as does sialyltransferase 4B. Two transcript variants encoding the same protein have been found for this gene. Other transcript variants may exist, but have not been fully characterized yet. Sialyltransferases expression and activity are increased in Grave's disease

### 11q13 ARCHEON

In line with our finding of the importance of “Amplified Regions of CHromosomal Expression obseved in Neoplasisa” (see above), the 11q13 ARCHEON displays functionally interacting genes involved in cell-growth (e.g. CCND1, FGF3, FGF4), apoptosis (e.g. TMEM16A, FADD) and cell adhesion (e.g. CCTN, SHANK2, PPFIA1).

### MYEOV

### Other Aliases: OCIM

Sequence analysis of MYEOV predicted a 313-amino acid protein that contains no known functional motifs except for an RNP1 motif typical of RNA-binding proteins and a leucine-isoleucine tail similar to cytoplasmically exposed membrane proteins with a C-terminal membrane anchor. Northern blot analysis detected a major 2.8-kb and a minor 3.5-kb transcript in various tumor cell lines. In 3 of 7 multiple myeloma cell lines with a t(11;14)(q13;q32) translocation and cyclin D1 overexpression, MYEOV was overexpressed. In all 7 cell lines, the breakpoint was mapped to the 360-kb region between the 2 genes. MYEOV overexpression was associated with the juxtaposition of an enhancer to the MYEOV gene. MYEOV gene has been mapped to 11q-13.1, 360 kb centromeric to CCND1.

DNA amplifications at 11q13 are frequently observed in esophageal squamous cell carcinoma and correlate with a malignant phenotype. Although this amplicon spans a region of several megabases and harbors numerous genes, CCND1 and EMS1 are thought to be the relevant candidates in esophageal carcinoma. It has been investigated whether the putative transforming gene MYEOV, mapping 360 kb centromeric to CCND1 and activated concomitantly with CCND1 in a subset of t(1;14)(q13;q32) positive multiple myeloma cell lines, represents a target of 11q13 amplification in esophageal carcinoma. MYEOV was always coamplified with CCND1. However, its activation was sometimes inhibited by an epigenetic mechanism and is associated with esophageal squamous cell carcinomas

### CCND1

### Other Aliases: BCL1, D11S287E, PRAD1, U21B31

CCND1 belongs to the highly conserved cyclin family, whose members are characterized by a dramatic periodicity in protein abundance throughout the cell cycle. Cyclins function as regulators of CDK kinases. Different cyclins exhibit distinct expression and degradation patterns which contribute to the temporal coordination of each mitotic event. This cyclin forms a complex with and functions as a regulatory subunit of CDK4 or CDK6, whose activity is required for cell cycle G1/S transition. This protein has been shown to interact with tumor suppressor protein Rb and the expression of this gene is regulated positively by Rb. Mutations, amplification and overexpression of this gene, which alters cell cycle progression, are observed frequently in a variety of tumors and may contribute to tumorigenesis. CCND1 is a target gene of the WNT signalling pathway. Expression levels of CCND1 predict the cellular effects of mTOR inhibitors. A marked intratumoral heterogeneity of c-myc and CCND1, but not of c-erbB2 amplification has been reported in breast cancer. CCND1 promoter activation by estrogens in human breast cancer cells is mediated by recruitment of a c-Jun/c-Fos/estrogen receptor alpha/progesterone receptor complex to the tetradecanoyl phorbol acetate-responsive element of the gene. Overexpression of cyclin D1 has been found to be significantly correlated with increased chromosomal instability in patients with breast cancer.

### ORAOV1

### Other Aliases: TAOS1

Mapping of the 11q13 amplicon has identified a gene that is amplified and overexpressed in oral cancer cells.

### FGF19

FGF19 is a member of the fibroblast growth factor (FGF) family. FGF family members possess broad mitogenic and cell survival activities, and are involved in a variety of biological processes including embryonic development cell growth, morphogenesis, tissue repair, tumor growth and invasion. This growth factor is a high affinity, heparin dependent ligand for FGFR4. Expression of this gene was detected only in fetal but not adult brain tissue. Synergistic interaction of the chick homolog and Wnt-8c has been shown to be required for initiation of inner ear development.

### FGF4

### Other Aliases: HBGF-4, HST, HST-1, HSTF1, K-FGF, KFGF

FGF4 is a member of the fibroblast growth factor (FGF) family. FGF family members possess broad mitogenic and cell survival activities and are involved in a variety of biological processes including embryonic development, cell growth, morphogenesis, tissue repair, tumor growth and invasion. This gene was identified by its oncogenic transforming activity. This gene and FGF3, another oncogenic growth factor, are located closely on chromosome 11. Co-amplification of both genes was found in various kinds of human tumors. Studies on the mouse homolog suggested a function in bone morphogenesis and limb development through the sonic hedgehog (SHH) signaling pathway.

FGF4 is a direct target of LEF1 and Wnt signaling during tooth development and limb outgrowth. Recombinant FGF4 protein could fully overcome the developmental arrest of tooth germs seen in Lef1-deficient mice. The FGF4 beads also induced delayed expression of Shh in the epithelium. It has been hypothesized that the sole function of LEF1 in odontogenesis may be to activate Fgf4 and to connect the Wnt and FGF signaling pathways at a specific developmental step.

### FGF3

### Other Aliases: HBGF-3, INT2

FGF3 is a member of the fibroblast growth factor (FGF) family. FGF family members possess broad mitogenic and cell survival activities and are involved in a variety of biological processes including embryonic development, cell growth, morphogenesis, tissue repair, tumor growth and invasion. FGF3 was identified by its similarity with mouse fgf3/int-2, a proto-oncogene activated in virally induced mammary tumors in the mouse. Frequent amplification of this gene has been found in human tumors, which may be important for neoplastic transformation and tumor progression. Studies of the similar genes in mouse and chicken suggested a role in inner ear formation.

### TMEM16A

### Other Aliases: FLJ10261, ORAOV2, TAOS2

TMEM16A is located within the CCND1-EMS1 locus on human chromosome 11q13 and encodes a eight-transmembrane protein homologous to C12orf3, C11orf25 and FLJ34272 gene products and is amplified in various cancers. We have found, that TMEM16A contains death domains and has functions within cell death regulation (apotosis).

### FADD

Gene aliases: GIG3; MORT1; MGC8528

Cell signalling pathways that regulate proliferation and those that regulate programmed cell death (apoptosis) are co-ordinated. The proteins and mechanisms that mediate the integration of these pathways are not yet fully described. FADD is an adaptor molecule that interacts with various cell surface receptors and mediates cell apoptotic signals. Through its C-terminal death domain, this protein can be recruited by TNFRSF6/Fas-receptor, tumor necrosis factor receptor, TNFRSF25, and TNFSF10/TRAIL-receptor, and thus it participates in the death signaling initiated by these receptors. Interaction of this protein with the receptors unmasks the N-terminal effector domain of this protein, which allows it to recruit caspase-8, and thereby activate the cysteine protease cascade. JNK-mediated phosphorylation of FADD plays an important role in the negative regulation of cell growth and metastasis, independent of the ER status of a breast cancer. The phosphoprotein PEA-15 (phosphoprotein enriched in astrocytes) can regulate both the ERK (extracellular-signal-regulated kinase)/MAPK (mitogen-activated protein kinase) pathway and the death receptor-initiated apoptosis pathway. This is the result of PEA-15 binding to the ERK/MAPK or the proapoptotic protein FADD (Fas-activated death domain protein) respectively. Phosphorylation of PEA-15 at SER-104 and SER-116 acts as the switch that controls whether PEA-15 influences proliferation or apoptosis.

### PPFIA1

Gene aliases: LIP1; LIP.1; LIPRIN; MGC26800

PPFIA1 is a member of the LAR protein-tyrosine phosphatase-interacting protein (liprin) family. Liprins interact with members of LAR family of transmembrane protein tyrosine phosphatases, which are known to be important for axon guidance and mammary gland development. This protein binds to the intracellular membrane-distal phosphatase domain of tyrosine phosphatase LAR, and appears to localize LAR to cell focal adhesions. This interaction may regulate the disassembly of focal adhesion and thus help orchestrate cell-matrix interactions. Alternatively spliced transcript variants encoding distinct isoforms have been described. Physical and functional interactions between protein tyrosine phosphatase alpha, PI 3-kinase, and PKCdelta have been shown. We have found that this gene seems to be important in cell growth and cell maintenance by involvement in chromosome segregation processes.

### CCTN

Gene aliases: EMS1; FLJ34459; cortactin

CCTN is overexpressed in breast cancer and squamous cell carcinomas of the head and neck. The encoded protein is localized in the cytoplasm and in areas of the cell-substratum contacts. This gene has two roles: (1) regulating the interactions between components of adherens-type junctions and (2) organizing the cytoskeleton and cell adhesion structures of epithelia and carcinoma cells. CCTN recruitment is dependent on the activation of a phosphoinositide-3-kinase/Rac1-GTPase signalling pathway, which is required for actin polymerization. CCTN mediates the invasive potential of human carcinomas and promotes cell motility by enhancing lamellipodial persistence, at least in part through regulation of Arp2/3 complex. Moreover, CCTN links receptor endocytosis to actin polymerization by binding both CD2AP and the Arp2/3 complex, which may facilitate the trafficking of internalized growth factor receptors. During apoptosis, the encoded protein is degraded in a caspase-dependent manner. The aberrant regulation of this gene contributes to tumor cell invasion and metastasis. Two splice variants that encode different isoforms have been identified for this gene. We have found, that PPFIA1 and CCTN are both functionally interacting to control cell adhesion within this ARCHEON, and that their function is negatively regulated by the apoptosis function of within this ARCHEON (TMEM16A and FADD).

### SHANK2

Gene aliases: SHANK; CORTBP1; CTTNBP1; ProSAP1; SPANK-3

SHANK2 is a member of the Shank family of synaptic proteins that may function as molecular scaffolds in the postsynaptic density (PSD). Shank proteins contain multiple domains for protein-protein interaction, including ankyrin repeats, an SH3 domain, a PSD-95/Dlg/ZO-1 domain, a sterile alpha motif domain, and a proline-rich region. This particular family member contains a PDZ domain, a consensus sequence for cortactin SH3 domain-binding peptides and a sterile alpha motif. The alternative splicing demonstrated in Shank genes has been suggested as a mechanism for regulating the molecular structure of Shank and the spectrum of Shank-interacting proteins in the PSDs of adult and developing brain. Two alternative splice variants, encoding distinct isoforms, are reported. Additional splice variants exist but their full-length nature has not been determined. Interestingly, SHANK 2 also physically interacts with its genomic neighbor CTTN in brain tissues and therefore has been named CTTNBP1. We have found, that CTTN and SHANK2 coexpression contributes to cell migration and regulation of cell adhesion in cancer.

### MLN50

By differential screening of cDNAs from breast cancer-derived metastatic axillary lymph nodes, TRAF4 and 3 other novel genes (MLN51, MLN62, MLN64) were identified that are overexpressed in breast cancer [Tomasetto et al., 1995, (3)]. One gene, which they designated MLN50, was mapped to 17q11-q21.3 by radioactive in situ hybridization. In breast cancer cell lines, overexpression of the 4 kb MLN50 mRNA was correlated with amplification of the gene and with amplification and overexpression of ERBB2, which maps to the same region. The authors suggested that the 2 genes belong to the same amplicon. Amplification of chromosomal region 17q11-q21 is one of the most common events occurring in human breast cancers. They reported that the predicted 261-amino acid MLN50 protein contains an N-terminal LIM domain and a C-terminal SH3 domain. They renamed the protein LASP1, for ‘LIM and SH3 protein.’ Northern blot analysis revealed that LASP1 mRNA was expressed at a basal level in all normal tissues examined and overexpressed in 8% of primary breast cancers. In most of these cancers, LASP1 and ERBB2 were simultaneously overexpressed.

### MLLT6

The MLLT6 (AF17) gene encodes a protein of 1,093 amino acids, containing a leucine-zipper dimerization motif located 3-prime of the fusion point and a cysteine-rich domain at the end terminus. AF17 was found to contain stretches of amino acids previously associated with domains involved in transcriptional repression or activation.

Chromosome translocations involving band 11q23 are associated with approximately 10% of patients with acute lymphoblastic leukemia (ALL) and more than 5% of patients with acute myeloid leukemia (AML). The gene at 11q23 involved in the translocations is variously designated ALL1, HRX, MLL, and TRX1. The partner gene in one of the rarer translocations, t(11;17)(q23;q21), designated MLLT6 on 17q12.

### ZNF144 (Mel18)

Mel18 cDNA encodes a novel cys-rich zinc finger motif. The gene is expressed strongly in most tumor cell lines, but its normal tissue expression was limited to cells of neural origin and was especially abundant in fetal neural cells. It belongs to a RING-finger motif family which includes BMI1. The MEL18/BMI1 gene family represents a mammalian homolog of the Drosophila ‘polycomb’ gene group, thereby belonging to a memory mechanism involved in maintaining the expression pattern of key regulatory factors such as Hox genes. Bmil, Mel18 and M33 genes, as representative examples of mouse Pc-G genes. Common phenotypes observed in knockout mice mutant for each of these genes indicate an important role for Pc-G genes not only in regulation of Hox gene expression and axial skeleton development but also in control of proliferation and survival of haematopoietic cell lineages. This is in line with the observed proliferative deregulation observed in lymphoblastic leukemia. The MEL18 gene is conserved among vertebrates. Its mRNA is expressed at high levels in placenta, lung, and kidney, and at lower levels in liver, pancreas, and skeletal muscle. Interestingly, cervical and lumbo-sacral-HOX gene expression is altered in several primary breast cancers with respect to normal breast tissue with the HoxB gene cluster being present on 17q distal to the 17q21 locus. Moreover, delay of differentiation with persistent nests of proliferating cells was found in endothelial cells cocultured with HOXB7-transduced SkBr3 cells, which exhibit a 17q21 amplification. Tumorigenicity of these cells has been evaluated in vivo. Xenograft in athymic nude mice showed that SkBr3/HOXB7 cells developed tumors with an increased number of blood vessels, either irradiated or not, whereas parental SkBr3 cells did not show any tumor take unless mice were sublethally irradiated. As part of this invention, we have found MEL18 to be overexpressed specifically in tumors bearing Her-2/neu gene amplification, which can be critical for Hox expression.

### PIP5K2B

Phosphoinositide kinases play central roles in signal transduction. Phosphatidylinositol-4-phosphate 5-kinases (PIP5Ks) phosphorylate phosphatidylinositol 4-phosphate, giving rise to phosphatidylinositol 4,5-bisphosphate. The PIP5K enzymes exist as multiple isoforms that have various immunoreactivities, kinetic properties, and molecular masses. They are unique in that they possess almost no homology to the kinase motifs present in other phosphatidylinositol, protein, and lipid kinases. By screening a human fetal brain cDNA library with the PIP5K2B EST the full length gene could be isolated. The deduced 416-amino acid protein is 78% identical to PIP5K2A. Using SDS-PAGE, the authors estimated that bacterially expressed PIP5K2B has a molecular mass of 47 kD. Northern blot analysis detected a 6.3-kb PIP5K2B transcript which was abundantly expressed in several human tissues. PIP5K2B interacts specifically with the juxtamembrane region of the p55 TNF receptor (TNFR1) and PIP5K2B activity is increased in mammalian cells by treatment with TNF-alpha. A modeled complex with membrane-bound substrate and ATP shows how a phosphoinositide kinase can phosphorylate its substrate in situ at the membrane interface. The substrate-binding site is open on 1 side, consistent with dual specificity for phosphatidylinositol 3- and 5-phosphates. Although the amino acid sequence of PIP5K2A does not show homology to known kinases, recombinant PIP5K2A exhibited kinase activity. PIP5K2A contains a putative Src homology 3 (SH3) domain-binding sequence. Overexpression of mouse PIP5K₁B in COS7 cells induced an increase in short actin fibers and a decrease in actin stress fibers.

### TEM7

Using serial analysis of gene expression (SAGE) a partial cDNAs corresponding to several tumor endothelial markers (TEMs) that displayed elevated expression during tumor angiogenesis could be identified. Among the genes identified was TEM7. Using database searches and 5-prime RACE the entire TEM7 coding region, which encodes a 500-amino acid type I transmembrane protein, has been described. The extracellular region of TEM7 contains a plexin-like domain and has weak homology to the ECM protein nidogen. The function of these domains, which are usually found in secreted and extracellular matrix molecules, is unknown. Nidogen itself belongs to the entactin protein family and helps to determine pathways of migrating axons by switching from circumferential to longitudinal migration. Entactin is involved in cell migration, as it promotes trophoblast outgrowth through a mechanism mediated by the RGD recognition site, and plays an important role during invasion of the endometrial basement membrane at implantation. As entactin promotes thymocyte adhesion but affects thymocyte migration only marginally, it is suggested that entactin may plays a role in thymocyte localization during T cell development.

In situ hybridization analysis of human colorectal cancer demonstrated that TEM7 was expressed clearly in the endothelial cells of the tumor stroma but not in the endothelial cells of normal colonic tissue. Using in situ hybridization to assay expression in various normal adult mouse tissues, they observed that TEM7 was largely undetectable in mouse tissues or tumors, but was abundantly expressed in mouse brain.

### ZNFN1A3

By screening a B-cell cDNA library with a mouse Aiolos N-terminal cDNA probe, a cDNA encoding human Aiolos, or ZNFN1A3, was obtained. The deduced 509-amino acid protein, which is 86% identical to its mouse counterpart, has 4 DNA-binding zinc fingers in its N terminus and 2 zinc fingers that mediate protein dimerization in its C terminus. These domains are 100% and 96% homologous to the corresponding domains in the mouse protein, respectively. Northern blot analysis revealed strong expression of a major 11.0- and a minor 4.4-kb ZNFN1A3 transcript in peripheral blood leukocytes, spleen, and thymus, with lower expression in liver, small intestine, and lung.

Ikaros (ZNFN1A1), a hemopoietic zinc finger DNA-binding protein, is a central regulator of lymphoid differentiation and is implicated in leukemogenesis. The execution of normal function of Ikaros requires sequence-specific DNA binding, transactivation, and dimerization domains. Mice with a mutation in a related zinc finger protein, Aiolos, are prone to B-cell lymphoma. In chemically induced murine lymphomas allelic losses on markers surrounding the Znfn1a1 gene were detected in 27% of the tumors analyzed. Moreover specific Ikaros expression was in primary mouse hormone-producing anterior pituitary cells and substantial for Fibroblast growth factor receptor 4 (FGFR4) expression, which itself is implicated in a multitude of endocrine cell hormonal and proliferative properties with FGFR4 being differentially expressed in normal and neoplastic pituitary. Moreover Ikaros binds to chromatin remodelling complexes containing SWI/SNF proteins, which antagonize Polycomb function. Interestingly at the telomeric end of the disclosed ARCHEON the SWI/SNF complex member SMARCE1 (=SWI/SNF-related, matrix-associated, actin-dependent regulators of chromatin) is located and part of the described amplification. Due to the related binding specificities of Ikaros and Palindrom Binding Protein (PBP) it is suggestive, that ZNFN1A3 is able to regulate the Her-2/neu enhancer.

### PPP1R1B

Midbrain dopaminergic neurons play a critical role in multiple brain functions, and abnormal signaling through dopaminergic pathways has been implicated in several major neurologic and psychiatric disorders. One well-studied target for the actions of dopamine is DARPP32. In the densely dopamine- and glutamate-innervated rat caudate-putamen, DARPP32 is expressed in medium-sized spiny neurons that also express dopamine D1 receptors. The function of DARPP32 seems to be regulated by receptor stimulation. Both dopaminergic and glutamatergic (NMDA) receptor stimulation regulate the extent of DARPP32 phosphorylation, but in opposite directions.

The human DARPP32 was isolated from a striatal cDNA library. The 204-amino acid DARPP32 protein shares 88% and 85% sequence identity, respectively, with bovine and rat DARPP32 proteins. The DARPP32 sequence is particularly conserved through the N terminus, which represents the active portion of the protein. Northern blot analysis demonstrated that the 2.1-kb DARPP32 mRNA is more highly expressed in human caudate than in cortex. In situ hybridization to postmortem human brain showed a low level of DARPP32 expression in all neocortical layers, with the strongest hybridization in the superficial layers. CDK5 phosphorylated DARPP32 in vitro and in intact brain cells. Phospho-thr75 DARPP32 inhibits PKA in vitro by a competitive mechanism. Decreasing phospho-thr75 DARPP32 in striatal cells either by a CDK5-specific inhibitor or by using genetically altered mice resulted in increased dopamine-induced phosphorylation of PKA substrates and augmented peak voltage-gated calcium currents. Thus, DARPP32 is a bifunctional signal transduction molecule which, by distinct mechanisms, controls a serine/threonine kinase and a serine/threonine phosphatase.

DARPP32 and t-DARPP are overexpressed in gastric cancers. It's suggested that overexpression of these 2 proteins in gastric cancers may provide an important survival advantage to neoplastic cells. It could be demonstrated that Darpp32 is an obligate intermediate in progesterone-facilitated sexual receptivity in female rats and mice. The facilitative effect of progesterone on sexual receptivity in female rats was blocked by antisense oligonucleotides to Darpp32. Homozygous mice carrying a null mutation for the Darpp32 gene exhibited minimal levels of progesterone-facilitated sexual receptivity when compared to their wildtype littermates, and progesterone significantly increased hypothalamic cAMP levels and cAMP-dependent protein kinase activity.

### CACNB 1

In 1991a cDNA clone encoding a protein with high homology to the beta subunit of the rabbit skeletal muscle dihydropyridine-sensitive calcium channel from a rat brain cDNA library [Pragnell et al., 1991, (4)]. This rat brain beta-subunit cDNA hybridized to a 3.4-kb message that was expressed in high levels in the cerebral hemispheres and hippocampus and much lower levels in cerebellum. The open reading frame encodes 597 amino acids with a predicted mass of 65,679 Da which is 82% homologous with the skeletal muscle beta subunit. The corresponding human beta-subunit gene was localized to chromosome 17 by analysis of somatic cell hybrids. The authors suggested that the encoded brain beta subunit, which has a primary structure highly similar to its isoform in skeletal muscle, may have a comparable role as an integral regulatory component of a neuronal calcium channel.

### RPL19

The ribosome is the only organelle conserved between prokaryotes and eukaryotes. In eukaryotes, this organelle consists of a 60S large subunit and a 40S small subunit. The mammalian ribosome contains 4 species of RNA and approximately 80 different ribosomal proteins, most of which appear to be present in equimolar amounts. In mammalian cells, ribosomal proteins can account for up to 15% of the total cellular protein, and the expression of the different ribosomal protein genes, which can account for up to 7 to 9% of the total cellular mRNAs, is coordinately regulated to meet the cell's varying requirements for protein synthesis. The mammalian ribosomal protein genes are members of multigene families, most of which are composed of multiple processed pseudogenes and a single functional intron-containing gene. The presence of multiple pseudogenes hampered the isolation and study of the functional ribosomal protein genes. By study of somatic cell hybrids, it has been elucidated that DNA sequences complementary to 6 mammalian ribosomal protein cDNAs could be assigned to chromosomes 5, 8, and 17. Ten fragments mapped to 3 chromosomes [Nakamichi et al., 1986, (5)]. These are probably a mixture of functional (expressed) genes and pseudogenes. One that maps to 5q23-q33 rescues Chinese hamster emetine-resistance mutations in interspecies hybrids and is therefore the transcriptionally active RPS14 gene. In 1989 a PCR-based strategy for the detection of intron-containing genes in the presence of multiple pseudogenes was described. This technique was used to identify the intron-containing PCR products of 7 human ribosomal protein genes and to map their chromosomal locations by hybridization to human/rodent somatic cell hybrids [Feo et al., 1992, (6)]. All 7 ribosomal protein genes were found to be on different chromosomes: RPL19 on 17p12-q11;RPL30 on 8; RPL35A on 18; RPL36A on 14; RPS6 on 9pter-p13; RPS11 on 19cen-qter; and RPS17 on 11pter-p13. These are also different sites from the chromosomal location of previously mapped ribosomal protein genes S14 on chromosome 5, S4 on Xq and Yp, and RP117A on 9q3-q34. By fluorescence in situ hybridization the position of the RPL19 gene was mapped to 17q11 [Davies et al., 1989, (7)].

### PPARBP

The thyroid hormone receptors (TRs) are hormone-dependent transcription factors that regulate expression of a variety of specific target genes. They must specifically interact with a number of proteins as they progress from their initial translation and nuclear translocation to hetero-dimerization with retinoid X receptors (RXRs), functional interactions with other transcription factors and the basic transcriptional apparatus, and eventually, degradation. To help elucidate the mechanisms that underlie the transcriptional effects and other potential functions of TRs, the yeast interaction trap, a version of the yeast 2-hybrid system, was used to identify proteins that specifically interact with the ligand-binding domain of rat TR-beta-1 (THRB) [Lee et al., 1995, (8)]. The authors isolated HeLa cell cDNAs encoding several different TR-interacting proteins (TRIPs), including TRIP2. TRIP2 interacted with rat Thrb only in the presence of thyroid hormone. It showed a ligand-independent interaction with RXR-alpha, but did not interact with the glucocorticoid receptor (NR3C1) under any condition. By immunoscreening a human B-lymphoma cell cDNA expression library with the anti-p53 monoclonal antibody PAb1801, PPARBP was identified, which was called RB18A for ‘recognized by PAb 1801 monoclonal antibody’ [Drane et al., 1997, (9)]. The predicted 1,566-amino acid RB18A protein contains several potential nuclear localization signals, 13 potential N-glycosylation sites, and a high number of potential phosphorylation sites. Despite sharing common antigenic determinants with p53, RB18A does not show significant nucleotide or amino acid sequence similarity with p53. Whereas the calculated molecular mass of RB18A is 166 kD, the apparent mass of recombinant RB18A was 205 kD by SDS-PAGE analysis. The authors demonstrated that RB18A shares functional properties with p53, including DNA binding, p53 binding, and self-oligomerization. Furthermore, RB18A was able to activate the sequence-specific binding of p53 to DNA, which was induced through an unstable interaction between both proteins. Northern blot analysis of human tissues detected an 8.5-kb RB18A transcript in all tissues examined except kidney, with highest expression in heart. Moreover mouse Pparbp, which was called Pbp for ‘Ppar-binding protein,’ as a protein that interacts with the Ppar-gamma (PPARG) ligand-binding domain in a yeast 2-hybrid system was identified [Zhu et al., 1997, (10)]. The authors found that Pbp also binds to PPAR-alpha (PPARA), RAR-alpha (RARA), RXR, and TR-beta-1 in vitro. The binding of Pbp to these receptors increased in the presence of specific ligands. Deletion of the last 12 amino acids from the C terminus of PPAR-gamma resulted in the abolition of interaction between Pbp and PPAR-gamma. Pbp modestly increased the transcriptional activity of PPAR-gamma, and a truncated form of Pbp acted as a dominant-negative repressor, suggesting that Pbp is a genuine transcriptional co-activator for PPAR. The predicted 1,560-amino acid Pbp protein contains 2 LXXLL motifs, which are considered necessary and sufficient for the binding of several co-activators to nuclear receptors. Northern blot analysis detected Pbp expression in all mouse tissues examined, with higher levels in liver, kidney, lung, and testis. In situ hybridization showed that Pbp is expressed during mouse ontogeny, suggesting a possible role for Pbp in cellular proliferation and differentiation. In adult mouse, in situ hybridization detected Pbp expression in liver, bronchial epithelium in the lung, intestinal mucosa, kidney cortex, thymic cortex, splenic follicles, and seminiferous epithelium in testis. Lateron PPARBP was identified, which was called TRAP220, from an immunopurified TR-alpha (THRA)-TRAP complex [Yuan et al., 1998, (11)]. The authors cloned Jurkat cell cDNAs encoding TRAP220. The predicted 1,581-amino acid TRAP220 protein contains LXXLL domains, which are found in other nuclear receptor-interacting proteins. TRAP220 is nearly identical to RB18A, with these proteins differing primarily by an extended N terminus on TRAP220. In the absence of TR-alpha, TRAP220 appears to reside in a single complex with other TRAPs. TRAP220 showed a direct ligand-dependent interaction with TR-alpha, which was mediated through the C terminus of TR-alpha and, at least in part, the LXXLL domains of TRAP220. TRAP220 also interacted with other nuclear receptors, including vitamin D receptor, RARA, RXRA, PPARA, PPARG, and estrogen receptor-alpha (ESR1; 133430), in a ligand-dependent manner. TRAP220 moderately stimulated human TR-alpha-mediated transcription in transfected cells, whereas a fragment containing the LXXLL motifs acted as a dominant-negative inhibitor of nuclear receptor-mediated transcription both in transfected cells and in cell-free transcription systems. Further studies indicated that TRAP220 plays a major role in anchoring other TRAPs to TR-alpha during the function of the TR-alpha-TRAP complex and that TRAP220 may be a global co-activator for the nuclear receptor superfamily. PBP, a nuclear receptor co-activator, interacts with estrogen receptor-alpha (ESR1) in the absence of estrogen. This interaction was enhanced in the presence of estrogen, but was reduced in the presence of the anti-estrogen Tamoxifen. Transfection of PBP into cultured cells resulted in enhancement of estrogen-dependent transcription, indicating that PBP serves as a co-activator in estrogen receptor signaling. To examine whether overexpression of PBP plays a role in breast cancer because of its co-activator function in estrogen receptor signaling, the levels of PBP expression in breast tumors was determined [Zhu et al., 1999, (12)]. High levels of PBP expression were detected in approximately 50% of primary breast cancers and breast cancer cell lines by ribonuclease protection analysis, in situ hybridization, and immunoperoxidase staining. By using FISH, the authors mapped the PBP gene to 17q12, a region that is amplified in some breast cancers. They found PBP gene amplification in approximately 24% (6 of 25) of breast tumors and approximately 30% (2 of 6) of breast cancer cell lines, implying that PBP gene overexpression can occur independent of gene amplification. They determined that the PBP gene comprises 17 exons that together span more than 37 kb. Their findings, in particular PBP gene amplification, suggested that PBP, by its ability to function as an estrogen receptor-alpha co-activator, may play a role in mammary epithelial differentiation and in breast carcinogenesis.

### NEUROD2

Basic helix-loop-helix (bHLH) proteins are transcription factors involved in determining cell type during development. In 1995 a bHLH protein was described, termed NeuroD (for ‘neurogenic differentiation’), that functions during neurogenesis [Lee et al., 1995, (13)]. The human NEUROD gene maps to chromosome 2q32. The cloning and characterization of 2 additional NEUROD genes, NEUROD2 and NEUROD3 was described in 1996 [McCormick et al., 1996, (14)]. Sequences for the mouse and human homologues were presented. NEUROD2 shows a high degree of homology to the bHLH region of NEUROD, whereas NEUROD3 is more distantly related. The authors found that mouse neuroD2 was initially expressed at embryonic day 11, with persistent expression in the adult nervous system. Similar to neuroD, neuroD2 appears to mediate neuronal differentiation. The human NEUROD2 was mapped to 17q12 by fluorescence in situ hybridization and the mouse homologue to chromosome 11 [Tamimi et al., 1997, (15)].

### Telethonin

Telethonin is a sarcomeric protein of 19 kD found exclusively in striated and cardiac muscle It appears to be localized to the Z disc of adult skeletal muscle and cultured myocytes. Telethonin is a substrate of titin, which acts as a molecular ‘ruler’ for the assembly of the sarcomere by providing spatially defined binding sites for other sarcomeric proteins. After activation by phosphorylation and calcium/calmodulin binding, titin phosphorylates the C-terminal domain of telethonin in early differentiating myocytes. The telethonin gene has been mapped to 17q12, adjacent to the phenylethanolamine N-methyltransferase gene [Valle et al., 1997, (16)].

### PENT, PNMT

Phenylethanolamine N-methyltransferase catalyzes the synthesis of epinephrine from norepinephrine, the last step of catecholamine biosynthesis. The cDNA clone was first isolated in 1998 for bovine adrenal medulla PNMT using mixed oligodeoxyribonucleotide probes whose synthesis was based on the partial amino acid sequence of tryptic peptides from the bovine enzyme [Kaneda et al., 1988, (17)]. Using a bovine cDNA as a probe, the authors screened a human pheochromocytoma cDNA library and isolated a cDNA clone with an insert of about 1.0 kb, which contained a complete coding region of the enzyme. Northern blot analysis of human pheochromocytoma polyadenylated RNA using this cDNA insert as the probe demonstrated a single RNA species of about 1,000 nucleotides, suggesting that this clone is a full-length cDNA. The nucleotide sequence showed that human PNMT has 282 amino acid residues with a predicted molecular weight of 30,853, including the initial methionine. The amino acid sequence was 88% homologous to that of bovine enzyme. The PNMT gene was found to consist of 3 exons and 2 introns spanning about 2,100 basepairs. It was demonstrated that in transgenic mice the gene is expressed in adrenal medulla and retina. A hybrid gene consisting of 2 kb of the PNMT 5-prime-flanking region fused to the simian virus 40 early region also resulted in tumor antigen mRNA expression in adrenal glands and eyes; furthermore, immunocytochemistry showed that the tumor antigen was localized in nuclei of adrenal medullary cells and cells of the inner nuclear cell layer of the retina, both prominent sites of epinephrine synthesis. The results indicate that the enhancer(s) for appropriate expression of the gene in these cell types are in the 2-kb 5-prime-flanking region of the gene.

Kaneda et al., 1988 (17), assigned the human PNMT gene to chromosome 17 by Southern blot analysis of DNA from mouse-human somatic cell hybrids. In 1992 the localization was narrowed down to 17q21-q22 by linkage analysis using RFLPs related to the PNMT gene and several 17q DNA markers [Hoehe et al., 1992, (18)]. The findings are of interest in light of the description of a genetic locus associated with blood pressure regulation in the stroke-prone spontaneously hypertensive rat (SHR-SP) on rat chromosome 10 in a conserved linkage synteny group corresponding to human chromosome 17q22-q24. See essential hypertension.

### MGC9753

This gene maps on chromosome 17, at 17q12 according to RefSeq. It is expressed at very high level. It is defined by cDNA clones and produces, by alternative splicing, 7 different transcripts can be obtained (SEQ ID NO:60 to 66 and 83 to 89,Table 1), altogether encoding 7 different protein isoforms. Of specific interest is the putatively secreted isoform g, encoded by a mRNA of 2.55 kb. It's premessenger covers 16.94 kb on the genome. It has a very long 3′ UTR. The protein (226 aa, MW 24.6 kDa, pI 8.5) contains no Pfam motif. The MGC9753 gene produces, by alternative splicing, 7 types of transcripts, predicted to encode 7 distinct proteins. It contains 13 confirmed introns, 10 of which are alternative. Comparison to the genome sequence shows that 11 introns follow the consensual [gt-ag] rule, 1 is atypical with good support [tg_cg]. The six most abundant isoforms are designated by a) to i) and code for proteins as follows:
a) This mRNA is 3.03 kb long, its premessenger covers 16.95 kb on the genome. It has a very long 3′ UTR. The protein (190 aa, MW 21.5 kDa, pI 7.2) contains no Pfam motif. It is predicted to localise in the endoplasmic reticulum.
c) This mRNA is 1.17 kb long, its premessenger covers 16.93 kb on the genome. It may be incomplete at the N terminus. The protein (368 aa, MW 41.5 kDa, pI 7.3) contains no Pfam motif.
d) This mRNA is 3.17 kb long, its premessenger covers 16.94 kb on the genome. It has a very long 3′ UTR and 5′p UTR. The protein (190 aa, MW 21.5 kDa, pI 7.2) contains no Pfam motif. It is predicted to localise in the endoplasmic reticulum.
g) This mRNA is 2.55 kb long, its premessenger covers 16.94 kb on the genome. It has a very long 3′ UTR. The protein (226 aa, MW 24.6 kDa, pI 8.5) contains no Pfam motif. It is predicted to be secreted.
h) This mRNA is 2.68 kb long, its premessenger covers 16.94 kb on the genome. It has a very long 3′ UTR. The protein (320 aa, MW 36.5 kDa, pI 6.8) contains no Pfam motif It is predicted to localise in the endoplasmic reticulum.
i) This mRNA is 2.34 kb long, its premessenger covers 16.94 kb on the genome. It may be incomplete at the N terminus. It has a very long 3′ UTR. The protein (217 aa, MW 24.4 kDa, pI 5.9) contains no Pfam motif.

The MCG9753 gene may be homologue to the CAB2 gene located on chromosome 17q12. The CAB2, a human homologue of the yeast COS16 required for the repair of DNA double-strand breaks was cloned. Autofluorescence analysis of cells transfected with its GFP fusion protein demonstrated that CAB2 translocates into vesicles, suggesting that overexpression of CAB2 may decrease intercellular Mn-

(2+) by accumulating it in the vesicles, in the same way as yeast.

### Her-2/neu

The oncogene originally called NEU was derived from rat neuro/glioblastoma cell lines. It encodes a tumor antigen, p185, which is serologically related to EGFR, the epidermal growth factor receptor. EGFR maps to chromosome 7. In1985 it was found, that the human homologue, which they designated NGL (to avoid confusion with neuraminidase, which is also symbolized NEU), maps to 17q12-q22 by in situ hybridization and to 17q21-qter in somatic cell hybrids [Yang-Feng et al., 1985, (19)]. Thus, the SRO is 17q21-q22. Moreover, in 1985 a potential cell surface receptor of the tyrosine kinase gene family was identified and characterized by cloning the gene [Coussens et al., 1985, (20)]. Its primary sequence is very similar to that of the human epidermal growth factor receptor. Because of the seemingly close relationship to the human EGF receptor, the authors called the gene HER2. By Southern blot analysis of somatic cell hybrid DNA and by in situ hybridization, the gene was assigned to 17q21-q22. This chromosomal location of the gene is coincident with the NEU oncogene, which suggests that the 2 genes may in fact be the same; indeed, sequencing indicates that they are identical. In 1988 a correlation between overexpression of NEU protein and the large-cell, comedo growth type of ductal carcinoma was found [van de Vijver et al., 1988, (21)]. The authors found no correlation, however, with lymph-node status or tumor recurrence. The role of HER2/NEU in breast and ovarian cancer was described in 1989, which together account for one-third of all cancers in women and approximately one-quarter of cancer-related deaths in females [Slamon et al., 1989, (22)].

An ERBB-related gene that is distinct from the ERBB gene, called ERBB1 was found in 1985. ERBB2 was not amplified in vulva carcinoma cells with EGFR amplification and did not react with EGF receptor mRNA. About 30-fold amplification of ERBB2 was observed in a human adenocarcinoma of the salivary gland. By chromosome sorting combined with velocity sedimentation and Southern hybridization, the ERBB2 gene was assigned to chromosome 17 [Fukushige et al., 1986, (23)]. By hybridization to sorted chromosomes and to metaphase spreads with a genomic probe, they mapped the ERBB2 locus to 17q21. This is the chromosome 17 breakpoint in acute promyelocytic leukemia (APL). Furthermore, they observed amplification and elevated expression of the ERBB2 gene in a gastric cancer cell line. Antibodies against a synthetic peptide corresponding to 14 amino acid residues at the COOH-terminus of a protein deduced from the ERBB2 nucleotide sequence were raised in 1986. With these antibodies, the ERBB2 gene product from adenocarcinoma cells was precipitated and demonstrated to be a 185-kD glycoprotein with tyrosine kinase activity. A cDNA probe for ERBB2 and by in situ hybridization to APL cells with a 15;17 chromosome translocation located the gene to the proximal side of the breakpoint [Kaneko et al., 1987, (24)]. The authors suggested that both the gene and the breakpoint are located in band 17q21.1 and, further, that the ERBB2 gene is involved in the development of leukemia. In 1987 experiments indicated that NEU and HER2 are both the same as ERBB2 [Di Fiore et al., 1987, (25)]. The authors demonstrated that overexpression alone can convert the gene for a normal growth factor receptor, namely, ERBB2, into an oncogene. The ERBB2 to 17q11-q21 by in situ hybridization [Popescu et al., 1989, (26)]. By in situ hybridization to chromosomes derived from fibroblasts carrying a constitutional translocation between 15 and 17, they showed that the ERBB2 gene was relocated to the derivative chromosome 15; the gene can thus be localized to 17q12-q21.32. By family linkage studies using multiple DNA markers in the 17q12-q21 region the ERBB2 gene was placed on the genetic map of the region.

Interleukin-6 is a cytokine that was initially recognized as a regulator of immune and inflammatory responses, but also regulates the growth of many tumor cells, including prostate cancer. Overexpression of ERBB2 and ERBB3 has been implicated in the neoplastic transformation of prostate cancer. Treatment of a prostate cancer cell line with IL6 induced tyrosine phosphorylation of ERBB2 and ERBB3, but not ERBB1/EGFR. The ERBB2 forms a complex with the gp130 subunit of the IL6 receptor in an IL6-dependent manner. This association was important because the inhibition of ERBB2 activity resulted in abrogation of IL6-induced MAPK activation. Thus, ERBB2 is a critical component of IL6 signaling through the MAP kinase pathway [Qiu et al., 1998, (27)]. These findings showed how a cytokine receptor can diversify its signaling pathways by engaging with a growth factor receptor kinase.

Overexpression of ERBB2 confers Taxol resistance in breast cancers. Overexpression of ERBB2 inhibits Taxol-induced apoptosis [Yu et al., 1998, (28)]. Taxol activates CDC2 kinase in MDA-MB-435 breast cancer cells, leading to cell cycle arrest at the G2/M phase and, subsequently, apoptosis. A chemical inhibitor of CDC2 and a dominant-negative mutant of CDC2 blocked Taxol-induced apoptosis in these cells. Overexpression of ERBB2 in MDA-MB-435 cells by transfection transcriptionally upregulates CDKN1A which associates with CDC2, inhibits Taxol-mediated CDC2 activation, delays cell entrance to G2/M phase, and thereby inhibits Taxol-induced apoptosis. In CDKN1A antisense-transfected MDA-MB-435 cells or in p21−/− MEF cells, ERBB2 was unable to inhibit Taxol-induced apoptosis. Therefore, CDKN1A participates in the regulation of a G2/M checkpoint that contributes to resistance to Taxol-induced apoptosis in ERBB2-overexpressing breast cancer cells.

A secreted protein of approximately 68 kD was described, designated herstatin, as the product of an alternative ERBB2 transcript that retains intron 8 [Doherty et al., 1999, (29)]. This alternative transcript specifies 340 residues identical to subdomains I and II from the extracellular domain of p185ERBB2, followed by a unique C-terminal sequence of 79 amino acids encoded by intron 8. The recombinant product of the alternative transcript specifically bound to ERBB2-transfected cells and was chemically crosslinked to p185ERBB2, whereas the intron-encoded sequence alone also bound with high affinity to transfected cells and associated with p185 solubilized from cell extracts. The herstatin mRNA was expressed in normal human fetal kidney and liver, but was at reduced levels relative to p185ERBB2 mRNA in carcinoma cells that contained an amplified ERBB2 gene. Herstatin appears to be an inhibitor of p185ERBB2, because it disrupts dimers, reduces tyrosine phosphorylation of p185, and inhibits the anchorage-independent growth of transformed cells that overexpress ERBB2. The HER2 gene is amplified and HER2 is overexpressed in 25 to 30% of breast cancers, increasing the aggressiveness of the tumor. Finally, it was found that a recombinant monoclonal antibody against HER2 increased the clinical benefit of first-line chemotherapy in metastatic breast cancer that overexpresses HER2 [Slamon et al., 2001, (30)].

### GRB7

Growth factor receptor tyrosine kinases (GF-RTKs) are involved in activating the cell cycle. Several substrates of GF-RTKs contain Src-homology 2 (SH2) and SH3 domains. SH2 domain-containing proteins are a diverse group of molecules important in tyrosine kinase signaling. Using the CORT (cloning of receptor targets) method to screen a high expression mouse library, the gene for murine Grb7, which encodes a protein of 535 amino acids, was isolated [Margolis et al., 1992, (31)]. GRB7 is homologous to ras-GAP (ras-GTPase-activating protein). It contains an SH2 domain and is highly expressed in liver and kidney. This gene defines the GRB7 family, whose members include the mouse gene Grb10 and the human gene GRB14.

A putative GRB7 signal transduction molecule and a GRB7V novel splice variant from an invasive human esophageal carcinoma was isolated [Tanaka et al., 1998, (32)]. Although both GRB7 isoforms shared homology with the Mig-10 cell migration gene of Caenorhabditis elegans, the GRB7V isoform lacked 88 basepairs in the C terminus; the resultant frameshift led to substitution of an SH2 domain with a short hydrophobic sequence. The wildtype GRB7 protein, but not the GRB7V isoform, was rapidly tyrosyl phosphorylated in response to EGF stimulation in esophageal carcinoma cells. Analysis of human esophageal tumor tissues and regional lymph nodes with metastases revealed that GRB7V was expressed in 40% of GRB7-positive esophageal carcinomas. GRB7V expression was enhanced after metastatic spread to lymph nodes as compared to the original tumor tissues. Transfection of an antisense GRB7 RNA expression construct lowered endogenous GRB7 protein levels and suppressed the invasive phenotype exhibited by esophageal carcinoma cells. These findings suggested that GRB7 isoforms are involved in cell invasion and metastatic progression of human esophageal carcinomas. By sequence analysis, The GRB7 gene was mapped to chromosome 17q21-q22, near the topoisomerase-2 gene [Dong et al., 1997, (33)]. GRB-7 is amplified in concert with HER2 in several breast cancer cell lines and that GRB-7 is overexpressed in both cell lines and breast tumors. GRB-7, through its SH2 domain, binds tightly to HER2 such that a large fraction of the tyrosine phosphorylated HER2 in SKBR-3 cells is bound to GRB-7 [Stein et al., 1994, (34)].

### GCSF3

Granulocyte colony-stimulating factor (or colony stimulating factor-3) specifically stimulates the proliferation and differentiation of the progenitor cells for granulocytes. The partial amino acid sequence of purified GCSF protein was determined, and by using oligonucleotides as probes, several GCSF cDNA clones were isolated from a human squamous carcinoma cell line cDNA library [Nagata et al., 1986, (35)]. Cloning of human GCSF cDNA shows that a single gene codes for a 177- or 180-amino acid mature protein of molecular weight 19,600. The authors found that the GCSF gene has 4 introns and that 2 different polypeptides are synthesized from the same gene by differential splicing of mRNA. The 2 polypeptides differ by the presence or absence of 3 amino acids. Expression studies indicate that both have authentic GCSF activity. A stimulatory activity from a glioblastoma multiform cell line being biologically and biochemically indistinguishable from GCSF produced by a bladder cell line was found in 1987. By somatic cell hybridization and in situ chromosomal hybridization, the GCSF gene was mapped to 17q11 in the region of the breakpoint in the 15;17 translocation characteristic of acute promyelocytic leukemia [Le Beau et al., 1987, (36)]. Further studies indicated that the gene is proximal to the said breakpoint and that it remains on the rearranged chromosome 17. Southern blot analysis using both conventional and pulsed field gel electrophoresis showed no rearranged restriction fragments. By use of a full-length cDNA clone as a hybridization probe in human-mouse somatic cell hybrids and in flow-sorted human chromosomes, the gene for GCSF was mapped to 17q21-q22 lateron

### THRA

Both human and mouse DNA have been demonstrated to have two distantly related classes of ERBA genes and that in the human genome multiple copies of one of the classes exist [Jansson et al., 1983, (37)]. A cDNA was isolated derived from rat brain messenger RNA on the basis of homology to the human thyroid receptor gene [Thompson et al., 1987, (38)]. Expression of this cDNA produced a high-affinity binding protein for thyroid hormones. Messenger RNA from this gene was expressed in tissue-specific fashion, with highest levels in the central nervous system and no expression in the liver. An increasing body of evidence indicated the presence of multiple thyroid hormone receptors. The authors suggested that there may be as many as 5 different but related loci. Many of the clinical and physiologic studies suggested the existence of multiple receptors. For example, patients had been identified with familial thyroid hormone resistance in which peripheral response to thyroid hormones is lost or diminished while neuronal functions are maintained. Thyroidologists recognize a form of cretinism in which the nervous system is severely affected and another form in which the peripheral functions of thyroid hormone are more dramatically affected.

The cDNA encoding a specific form of thyroid hormone receptor expressed in human liver, kidney, placenta, and brain was isolated [Nakai et al., 1988, (39)]. Identical clones were found in human placenta. The cDNA encodes a protein of 490 amino acids and molecular mass of 54,824. Designated thyroid hormone receptor type alpha-2 (THRA2), this protein is represented by mRNAs of different size in liver and kidney, which may represent tissue-specific processing of the primary transcript.

The THRA gene contains 10 exons spanning 27 kb of DNA. The last 2 exons of the gene are alternatively spliced. A 5-kb THRA1 mRNA encodes a predicted 410-amino acid protein; a 2.7-kb THRA2 mRNA encodes a 490-amino acid protein. A third isoform, TR-alpha-3, is derived by alternative splicing. The proximal 39 amino acids of the TH-alpha-2 specific sequences are deleted in TR-alpha-3. A second gene, THRB on chromosome 3, encodes 2 isoforms of TR-beta by alternative splicing. In 1989 the structure and function of the EAR1 and EAR7 genes was elucidated, both located on 17q21 [Miyajima et al., 1989, (40)]. The authors determined that one of the exons in the EAR7 coding sequence overlaps an exon of EAR1, and that the 2 genes are transcribed from opposite DNA strands. In addition, the EAR7 mRNA generates 2 alternatively spliced isoforms, referred to as EAR71 and EAR72, of which the EAR71 protein is the human counterpart of the chicken c-erbA protein.

The thyroid hormone receptors, beta, alpha-1, and alpha-2 3 mRNAs are expressed in all tissues examined and the relative amounts of the three mRNAs were roughly parallel. None of the 3 mRNAs was abundant in liver, which is the major thyroid hormone-responsive organ. This led to the assumption that another thyroid hormone receptor may be present in liver. It was found that ERBA, which potentiates ERBB, has an amino acid sequence different from that of other known oncogene products and related to those of the carbonic anhydrases [Debuire et al., 1984, (41)]. ERBA potentiates ERBB by blocking differentiation of erythroblasts at an immature stage. Carbonic anhydrases participate in the transport of carbon dioxide in erythrocytes. In 1986 it was shown that the ERBA protein is a high-affinity receptor for thyroid hormone. The cDNA sequence indicates a relationship to steroid-hormone receptors, and binding studies indicate that it is a receptor for thyroid hormones. It is located in the nucleus, where it binds to DNA and activates transcription.

Maternal thyroid hormone is transferred to the fetus early in pregnancy and is postulated to regulate brain development. The ontogeny of TR isoforms and related splice variants in 9 first-trimester fetal brains by semi-quantitative RT-PCR analysis has been investigated. Expression of the TR-beta-1, TR-alpha-1, and TR-alpha-2 isoforms was detected from 8.1 weeks' gestation. An additional truncated species was detected with the TR-alpha-2 primer set, consistent with the TR-alpha-3 splice variant described in the rat. All TR-alpha-derived transcripts were coordinately expressed and increased approximately 8-fold between 8.1 and 13.9 weeks' gestation. A more complex ontogenic pattern was observed for TR-beta-1, suggestive of a nadir between 8.4 and 12.0 weeks' gestation. The authors concluded that these findings point to an important role for the TR-alpha-1 isoform in mediating maternal thyroid hormone action during first-trimester fetal brain development.

The identification of the several types of thyroid hormone receptor may explain the normal variation in thyroid hormone responsiveness of various organs and the selective tissue abnormalities found in the thyroid hormone resistance syndromes. Members of sibships, who were resistant to thyroid hormone action, had retarded growth, congenital deafness, and abnormal bones, but had normal intellect and sexual maturation, as well as augmented cardiovascular activity. In this family abnormal T3 nuclear receptors in blood cells and fibroblasts have been demonstrated.

The availability of cDNAs encoding the various thyroid hormone receptors was considered useful in determining the underlying genetic defect in this family.

The ERBA oncogene has been assigned to chromosome 17. The ERBA locus remains on chromosome 17 in the t(15;17) translocation of acute promyelocytic leukemia (APL). The thymidine kinase locus is probably translocated to chromosome 15; study of leukemia with t(17;21) and apparently identical breakpoint showed that TK was on 21q+. By in situ hybridization of a cloned DNA probe of c-erb-A to meiotic pachytene spreads obtained from uncultured spermatocytes it has been concluded that ERBA is situated at 17q21.33-17q22, in the same region as the break that generated the t(15;17) seen in APL. Because most of the grains were seen in 17q22, they suggested that ERBA is probably in the proximal region of 17q22 or at the junction between 17q22 and 17q21.33. By in situ hybridization it has been demonstrated, that that ERBA remains at 17q11-q12 in APL, whereas TP53, at 17q21-q22, is translocated to chromosome 15. Thus, ERBA must be at 17q11.2 just proximal to the breakpoint in the APL translocation and just distal to it in the constitutional translocation.

The aberrant THRA expression in nonfunctioning pituitary tumors has been hypothesized to reflect mutations in the receptor coding and regulatory sequences. They screened THRA mRNA and THRB response elements and ligand-binding domains for sequence anomalies. Screening THRA mRNA from 23 tumors by RNAse mismatch and sequencing candidate fragments identified 1 silent and 3 missense mutations, 2 in the common THRA region and 1 that was specific for the alpha-2 isoform. No THRB response element differences were detected in 14 nonfunctioning tumors, and no THRB ligand-binding domain differences were detected in 23 nonfunctioning tumors. Therefore it has been suggested that the novel thyroid receptor mutations may be of functional significance in terms of thyroid receptor action, and further definition of their functional properties may provide insight into the role of thyroid receptors in growth control in pituitary cells.

### RARA

A cDNA encoding a protein that binds retinoic acid with high affinity has been cloned [Petkovich et al., 1987, (42)]. The protein was found to be homologous to the receptors for steroid hormones, thyroid hormones, and vitamin D3, and appeared to be a retinoic acid-inducible transacting enhancer factor. Thus, the molecular mechanisms of the effect of vitamin A on embryonic development, differentiation and tumor cell growth may be similar to those described for other members of this nuclear receptor family. In general, the DNA-binding domain is most highly conserved, both within and between the 2 groups of receptors (steroid and thyroid); Using a cDNA probe, the RAR-alpha gene has been mapped to 17q21 by in situ hybridization [Mattei et al., 1988, (43)]. Evidence has been presented for the existence of 2 retinoic acid receptors, RAR-alpha and RAR-beta, mapping to chromosome 17q21.1 and 3p24, respectively. The alpha and beta forms of RAR were found to be more homologous to the 2 closely related thyroid hormone receptors alpha and beta, located on 17q11.2 and 3p25-p21, respectively, than to any other members of the nuclear receptor family. These observations suggest that the thyroid hormone and retinoic acid receptors evolved by gene, and possibly chromosome, duplications from a common ancestor, which itself diverged rather early in evolution from the common ancestor of the steroid receptor group of the family. They noted that the counterparts of the human RARA and RARB genes are present in both the mouse and chicken. The involvement of RARA at the APL breakpoint may explain why the use of retinoic acid as a therapeutic differentiation agent in the treatment of acute myeloid leukemias is limited to APL. Almost all patients with APL have a chromosomal translocation t(15;17)(q22;q21). Molecular studies reveal that the translocation results in a chimeric gene through fusion between the PML gene on chromosome 15 and the RARA gene on chromosome 17. A hormone-dependent interaction of the nuclear receptors RARA and RXRA with CLOCK and MOP4 has been presented.

### CDC6

In yeasts, Cdc6 (Saccharomyces cerevisiae) and Cdc18 (Schizosaccharomyces pombe) associate with the origin recognition complex (ORC) proteins to render cells competent for DNA replication. Thus, Cdc6 has a critical regulatory role in the initiation of DNA replication in yeast. cDNAs encoding Xenopus and human homologues of yeast CDC6 have been isolated [Williams et al., 1997, (44)]. They designated the human and Xenopus proteins p62(cdc6). Independently, in a yeast 2-hybrid assay using PCNA as bait, cDNAs encoding the human CDC6/Cdc18 homologue have been isolated [Saha et al, 1998, (45)]. These authors reported that the predicted 560-amino acid human protein shares approximately 33% sequence identity with the 2 yeast proteins. On Western blots of HeLa cell extracts, human CDC6/cdc 18 migrates as a 66-kD protein. Although Northern blots indicated that CDC6/Cdc18 mRNA levels peak at the onset of S phase and diminish at the onset of mitosis in HeLa cells, the authors found that total CDC6/Cdc 18 protein level is unchanged throughout the cell cycle. Immunofluorescent analysis of epitope-tagged protein revealed that human CDC6/Cdc18 is nuclear in G1- and cytoplasmic in S-phase cells, suggesting that DNA replication may be regulated by either the translocation of this protein between the nucleus and cytoplasm or by selective degradation of the protein in the nucleus. Immunoprecipitation studies showed that human CDC6/Cdc18 associates in vivo with cyclin A, CDK2, and ORC1. The association of cyclin-CDK2 with CDC6/Cdc18 was specifically inhibited by a factor present in mitotic cell extracts. Therefore it has been suggested that if the interaction between CDC6/Cdc18 with the S phase-promoting factor cyclin-CDK2 is essential for the initiation of DNA replication, the mitotic inhibitor of this interaction could prevent a premature interaction until the appropriate time in G1. Cdc6 is expressed selectively in proliferating but not quiescent mammalian cells, both in culture and within tissues in intact animals [Yan et al., 1998, (46)]. During the transition from a growth-arrested to a proliferative state, transcription of mammalian Cdc6 is regulated by E2F proteins, as revealed by a functional analysis of the human Cdc6 promoter and by the ability of exogenously expressed E2F proteins to stimulate the endogenous Cdc6 gene. Immunodepletion of Cdc6 by microinjection of anti-Cdc6 antibody blocked initiation of DNA replication in a human tumor cell line. The authors concluded that expression of human Cdc6 is regulated in response to mitogenic signals through transcriptional control mechanisms involving E2F proteins, and that Cdc6 is required for initiation of DNA replication in mammalian cells.

Using a yeast 2-hybrid system, co-purification of recombinant proteins, and immunoprecipitation, it has been demonstrated lateron that an N-terminal segment of CDC6 binds specifically to PR48, a regulatory subunit of protein phosphatase 2A (PP2A). The authors hypothesized that dephosphorylation of CDC6 by PP2A, mediated by a specific interaction with PR48 or a related B-double prime protein, is a regulatory event controlling initiation of DNA replication in mammalian cells. By analysis of somatic cell hybrids and by fluorescence in situ hybridization the human p62(cdc6) gene has been to 17q21.3.

### TOP2A

DNA topoisomerases are enzymes that control and alter the topologic states of DNA in both prokaryotes and eukaryotes. Topoisomerase II from eukaryotic cells catalyzes the relaxation of supercoiled DNA molecules, catenation, decatenation, knotting, and unknotting of circular DNA. It appears likely that the reaction catalyzed by topoisomerase II involves the crossing-over of 2 DNA segments. It has been estimated that there are about 100,000 molecules of topoisomerase II per HeLa cell nucleus, constituting about 0.1% of the nuclear extract. Since several of the abnormal characteristics of ataxia-telangiectasia appear to be due to defects in DNA processing, screening for these enzyme activities in 5 AT cell lines has been performed [Singh et al., 1988, (47)]. In comparison to controls, the level of DNA topoisomerase II, determined by unknotting of P4 phage DNA, was reduced substantially in 4 of these cell lines and to a lesser extent in the fifth. DNA topoisomerase I, assayed by relaxation of supercoil DNA, was found to be present at normal levels.

The entire coding sequence of the human TOP2 gene has been determined [Tsai-Pflugfelder et al., 1988, (48)].

In addition human cDNAs that had been isolated by screening a cDNA library derived from a mechlorethamine-resistant Burkitt lymphoma cell line (Raji-HN2) with a Drosophila Topo II cDNA had been sequenced [Chung et al., 1989, (49)]. The authors identified 2 classes of sequence representing 2 TOP2 isoenzymes, which have been named TOP2A and TOP2B. The sequence of 1 of the TOP2A cDNAs is identical to that of an internal fragment of the TOP2 cDNA isolated by Tsai-Pflugfelder et al., 1988 (48). Southern blot analysis indicated that the TOP2A and TOP2B cDNAs are derived from distinct genes. Northern blot analysis using a TOP2A-specific probe detected a 6.5-kb transcript in the human cell line U937. Antibodies against a TOP2A peptide recognized a 170-kD protein in U937 cell lysates. Therefore it was concluded that their data provide genetic and immunochemical evidence for 2 TOP2 isozymes. The complete structures of the TOP2A and TOP2B genes has been reported [Lang et al., 1998, (50)]. The TOP2A gene spans approximately 30 kb and contains 35 exons.

Tsai-Pflugfelder et al., 1988 (48) showed that the human enzyme is encoded by a single-copy gene which they mapped to 17q21-q22 by a combination of in situ hybridization of a cloned fragment to metaphase chromosomes and by Southern hybridization analysis with a panel of mouse-human hybrid cell lines. The assignment to chromosome 17 has been confirmed by the study of somatic cell hybrids. Because of co-amplification in an adenocarcinoma cell line, it was concluded that the TOP2A and ERBB2 genes may be closely linked on chromosome 17 [Keith et al., 1992, (51)]. Using probes that detected RFLPs at both the TOP2A and TOP2B loci, the demonstrated heterozygosity at a frequency of 0.17 and 0.37 for the alpha and beta loci, respectively. The mouse homologue was mapped to chromosome 11 [Kingsmore et al., 1993, (52)]. The structure and function of type II DNA topoisomerases has been reviewed [Watt et al., 1994, (53)]. DNA topoisomerase II-alpha is associated with the pol II holoenzyme and is a required component of chromatin-dependent co-activation. Specific inhibitors of topoisomerase II blocked transcription on chromatin templates, but did not affect transcription on naked templates. Addition of purified topoisomerase II-alpha reconstituted chromatin-dependent activation activity in reactions with core pol II. Therefore the transcription on chromatin templates seems to result in the accumulation of superhelical tension, making the relaxation activity of topoisomerase II essential for productive RNA synthesis on nucleosomal DNA.

### IGFBP4

Six structurally distinct insulin-like growth factor binding proteins have been isolated and their cDNAs cloned: IGFBP1, IGFBP2, IGFBP3, IGFBP4, IGFBP5 and IGFBP6. The proteins display strong sequence homologies, suggesting that they are encoded by a closely related family of genes. The IGFBPs contain 3 structurally distinct domains each comprising approximately one-third of the molecule. The N-terminal domain 1 and the C-terminal domain 3 of the 6 human IGFBPs show moderate to high levels of sequence identity including 12 and 6 invariant cysteine residues in domains 1 and 3, respectively (IGFBP6 contains 10 cysteine residues in domain 1), and are thought to be the IGF binding domains. Domain 2 is defined primarily by a lack of sequence identity among the 6 IGFBPs and by a lack of cysteine residues, though it does contain 2 cysteines in IGFBP4. Domain 3 is homologous to the thyroglobulin type I repeat unit. Recombinant human insulin-like growth factor binding proteins 4, 5, and 6 have been characterized by their expression in yeast as fusion proteins with ubiquitin [Kiefer et al., 1992, (54)]. Results of the study suggested to the authors that the primary effect of the 3 proteins is the attenuation of IGF activity and suggested that they contribute to the control of IGF-mediated cell growth and metabolism. Moreover, IGFBPs have influence on EGFR and Her-2/neu mediated signalling. Addition of IGFBPs to Her-2/neu overexpressing cells at least in part blocks growth and survival characteristica of the respective cells.

Based on peptide sequences of a purified insulin-like growth factor-binding protein (IGFBP) rat IGFBP4 has been cloned by using PCR [Shimasaki et al., 1990, (55)]. They used the rat cDNA to clone the human ortholog from a liver cDNA library. Human IGFBP4 encodes a 258-amino acid polypeptide, which includes a 21-amino acid signal sequence. The protein is very hydrophilic, which may facilitate its ability as a carrier protein for the IGFs in blood. Northern blot analysis of rat tissues revealed expression in all tissues examined, with highest expression in liver. It was stated that IGFBP4 acts as an inhibitor of IGF-induced bone cell proliferation. The genomic region containing the IGFBP gene. The gene consists of 4 exons spanning approximately 15 kb of genomic DNA has been examined [Zazzi et al., 1998, (56)]. The upstream region of the gene contains a TATA box and a cAMP-responsive promoter.

By in situ hybridization, the IGFBP4 gene was mapped to 17q12-q21 [Bajalica et al., 1992, (57)]. Because the hereditary breast-ovarian cancer gene BRCA1 had been mapped to the same region, it has been investigated whether IGFBP4 is a candidate gene by linkage analysis of 22 BRCA1 families; the finding of genetic recombination suggested that it is not the BRCA1 gene [Tonin et al., 1993, (58)].

### CCR7

Using PCR with degenerate oligonucleotides, a lymphoid-specific member of the G protein-coupled receptor family has been identified and mapped to 17q12-q21.2 by analysis of human/mouse somatic cell hybrid DNAs and fluorescence in situ hybridization. It has been shown that this receptor had been independently identified as the Epstein-Barr-induced cDNA (symbol EBI1) [Birkenbach et al., 1993, (59)]. EBI1 is expressed in normal lymphoid tissues and in several B- and T-lymphocyte cell lines. While the function and the ligand for EBI1 remains unknown, its sequence and gene structure suggest that it is related to receptors that recognize chemoattractants, such as interleukin-8, RANTES, C5a, and fMet-Leu-Phe. Like the chemoattractant receptors, EBI1 contains intervening sequences near its 5-prime end; however, EBI1 is unique in that both of its introns interrupt the coding region of the first extracellular domain. Mouse Ebi1 cDNA has been isolated and found to encode a protein with 86% identity to the human homologue.

Subsets of murine CD4+ T cells localize to different areas of the spleen after adoptive transfer. Naive and T helper-1 (TH1) cells, which express CCR7, home to the periarteriolar lymphoid sheath, whereas activated TH2 cells, which lack CCR7, form rings at the periphery of the T-cell zones near B-cell follicles. It has been found that retroviral transduction of TH2 cells with CCR7 forced them to localize in a TH1-like pattern and inhibited their participation in B-cell help in vivo but not in vitro. Apparently differential expression of chemokine receptors results in unique cellular migration patterns that are important for effective immune responses.

CCR7 expression divides human memory T cells into 2 functionally distinct subsets. CCR7-memory cells express receptors for migration to inflamed tissues and display immediate effector function. In contrast, CCR7⁺ memory cells express lymph node homing receptors and lack immediate effector function, but efficiently stimulate dendritic cells and differentiate into CCR7⁻-effector cells upon secondary stimulation. The CCR7⁺ and CCR7⁻ T cells, named central memory (T-CM) and effector memory (T-EM), differentiate in a step-wise fashion from naive T cells, persist for years after immunization, and allow a division of labor in the memory response.

CCR7 expression in memory CD8⁺ T lymphocyte responses to HIV and to cytomegalovirus (CMV) tetramers has been evaluated. Most memory T lymphocytes express CD45RO, but a fraction express instead the CD45RA marker. Flow cytometric analyses of marker expression and cell division identified 4 subsets of HIV- and CMV-specific CD8⁺ T cells, representing a lineage differentiation pattern: CD45RA+CCR7⁺ (double-positive); CD45RA⁻CCR7⁺; CD45RA⁻CCR7⁻-(double-negative); CD45RA⁺CCR7⁻. The capacity for cell division, as measured by 5-(and 6-)carboxyl-fluorescein diacetate, succinimidyl ester, and intracellular staining for the Ki67 nuclear antigen, is largely confined to the CCR7+ subsets and occurred more rapidly in cells that are also CD45RA⁺. Although the double-negative cells did not divide or expand after stimulation, they did revert to positivity for either CD45RA or CCR7 or both. The CD45RA⁺CCR7⁻ cells, considered to be terminally differentiated, fail to divide, but do produce interferon-gamma and express high levels of perforin. The representation of subsets specific for CMV and for HIV is distinct. Approximately 70% of HIV-specific CD8⁺ memory T cells are double-negative or preterminally differentiated compared to 40% of CMV-specific cells. Approximately 50% of the CMV-specific CD8+ memory T cells are terminally differentiated compared to fewer than 10% of the HIV-specific cells. It has been proposed that terminally differentiated CMV-specific cells are poised to rapidly intervene, while double-positive precursor cells remain for expansion and replenishment of the effector cell pool. Furthermore, high-dose antigen tolerance and the depletion of HIV-specific CD4⁺ helper T-cell activity may keep the HIV-specific memory CD8⁺ T cells at the double-negative stage, unable to differentiate to the terminal effector state. B lymphocytes recirculate between B cell-rich compartments (follicles or B zones) in secondary lymphoid organs, surveying for antigen. After antigen binding, B cells move to the boundary of B and T zones to interact with T-helper cells. Furthermore it has been demonstrated that antigen-engaged B cells have increased expression of CCR7, the receptor for the T-zone chemokines CCL19 (also known as ELC) and CCL21, and that they exhibit increased responsiveness to both chemoattractants. In mice lacking lymphoid CCL19 and CCL21 chemokines, or with B cells that lack CCR7, antigen engagement fails to cause movement to the T zone. Using retroviral-mediated gene transfer, the authors demonstrated that increased expression of CCR7 is sufficient to direct B cells to the T zone. Reciprocally, overexpression of CXCR5, the receptor for the B-zone chemokine CXCL13, is sufficient to overcome antigen-induced B-cell movement to the T zone. This points toward a mechanism of B-cell relocalization in response to antigen, and established that cell position in vivo can be determined by the balance of responsiveness to chemoattractants made in separate but adjacent zones.

### SMARCE 1

The SWI/SNF complex in S. cerevisiae and Drosophila is thought to facilitate transcriptional activation of specific genes by antagonizing chromatin-mediated transcriptional repression. The complex contains an ATP-dependent nucleosome disruption activity that can lead to enhanced binding of transcription factors. The BRG1/brm-associated factors, or BAF, complex in mammals is functionally related to SWI/SNF and consists of 9 to 12 subunits, some of which are homologous to SWI/SNF subunits. A 57-kD BAF subunit, BAF57, is present in higher eukaryotes, but not in yeast. Partial coding sequence has been obtained from purified BAF57 from extracts of a human cell line [Wang et al., 1998, (60)]. Based on the peptide sequences, they identified cDNAs encoding BAF57. The predicted 411-amino acid protein contains an HMG domain adjacent to a kinesin-like region. Both recombinant BAF57 and the whole BAF complex bind 4-way junction (4WJ) DNA, which is thought to mimic the topology of DNA as it enters or exits the nucleosome. The BAF57 DNA-binding activity has characteristics similar to those of other HMG proteins. It was found that complexes with mutations in the BAF57 HMG domain retain their DNA-binding and nucleosome-disruption activities. They suggested that the mechanism by which mammalian SWI/SNF-like complexes interact with chromatin may involve recognition of higher-order chromatin structure by 2 or more DNA-binding domains. RNase protection studies and Western blot analysis revealed that BAF57 is expressed ubiquitously. Several lines of evidence point toward the involvement of SWI/SNF factors in cancer development [Klochendler-Yeivin et al., 2002, (61)]. Moreover, SWI/SNF related genes are assigned to chromosomal regions that are frequently involved in somatic rearrangements in human cancers [Ring et al., 1998, (62)]. In this respect it is interesting that some of the SWI/SNF family members (i.e. SMARCC1, SMARCC2, SMARCD1 and SMARCD22 are neighboring 3 of the eucaryotic ARCHEONs we have identified (i.e. 3p21-p24, 12q13-q14 and 17q respectively) and which are part of the present invention. In this invention we could also map SMARCE1/BAF57 to the 17q12 region by PCR karyotyping.

The measurement of HER-2 gene expression by TaqMan PCR is a highly reproducible alternative approach for the determination of the HER-2 status in parrafin-embedded tissue from core-needle biopsies. The technical standardization allows a fast, automated evaluation of the HER-2 DNA amplification status in combination with RNA expression levels of genes that may be affected by the genomic alteration of the 17q12 ARCHEON, including genes such as Retinoic Acid Receptor alpha and Topo II alpha. The combination of IHC, FISH and TaqMan PCR therefore improves the selection of patients who benefit from a trastuzumab containing therapy. Moreover, we have found great discrepancies between DNA copy number and RNA expression level of the genes, that are thought to be the critical players in this region (i.e. Her-2/neu, c-Myc and CCND1). In particular it turned out, that there is no strict concordance between the expression level and the genomic status of c-Myc, as multiple tumors did exhibit high RNA expression level of c-Myc without exhibiting genomic alterations of this region. Moreover, c-Myc RNA expression alone did not correlate with clinical outcome. However, by using a hierarchical clustering method on basis of the 17q12 and 8q24 ARCHEONs four tumor subgroups could be identified. Two of the subgroups did contain the patients with most favorable outcome (i.e. ypCR and ypNO). Most interestingly, we have found that the simultaneous RNA expression of c-Myc and another 8q24 ARCHEON gene, which is downstream of RTK signalling pathways, identifies patients with favorable outcome within one of these subgroups. Moreover, the other tumor subgroup containing patients with pCR did not exhibit c-myc expression and does not seem to harbor c-myc amplified tumors. We conclude that the overexpression of c-Myc itself is not critical for response to neoadjuvant chemotherapy containing trastuzumab, but that the expression of other ARCHEON genes on the 8q24 is relevant. We have found that in addition to c-Myc the alteration of the 8q24 ARCHEON elevates the expression of downstream target of Her-2/neu, which in turn may increase the dependency to this signalling pathway. This may explain the increased dependence to Her-2/neu signalling and increased sensitivity to trastuzumab. Moreover we can demonstrate hereby, that the combined analysis of multiple genes within the same and other ARCHEON regions is superior to analysis of isolated genes of these regions. Also it enables the RNA detection of genomic alterations in case the expression of individual genes (as demonstrated by c-myc) is also present in tumors not bearing respective genomic alterations by determining more than one gene of this region by RNA analysis (as demonstrated by TRIB1 and c-Myc expression analysis.

Data of 155 tumor samples were analyzed. All tumors allegedly were of about 2 cm size before treatment (T2). Tumors “in situ” after therapy were excluded from the analysis. Tumors missing data in either tumor size (the target quantity) or expression in one or more of the following genes: MGC9753, c-Myc, ER, TRIB-1 were additionally excluded. This yielded a data basis of 53 valid samples. The greatest portion of the excluded tumors was missing a tumor size information after treatment. Within this subcohort 17% of tumors revealed a pathological confirmed complete response (ypCR). One predefined working hypothesis under consideration was that, if we have a tumor with elevated expression of c-Myc and Trib-1 (all compared to a copy number of 90), and a lowered expression of ER (compared to a value of 90), then the tumor is more likely to respond to the therapy (Herceptin) than a tumor that violates any of these conditions. It is important to keep in mind, that this algorithm was defined to predict response to trastuzumab itself, not to chemotherapy (see below). Here, a response to the therapy was defined as a post-treatment tumor size of T0 or T1 whereas no response was a tumor size of T2 or above. By using these response criteria 56% of the patients revealed a clinical response (“Complete Response” or “Partial Response”) to neoadjuvant treatment of trastuzumab combined with chemotherapy.

The original data yielded the following result

| | In | Out |
|---|---|---|
| Response | 7 | 22 |
| No Response | 1 | 23 |
| T0 | 4 | 5 |
| T1 | 3 | 17 |
| T2 | 1 | 15 |
| T3 | 0 | 4 |
| T4 | 0 | 4 |
| ypCR | 50% | 11% |
| ypN0 | 100% | 42% |

“In” here denotes samples fulfilling all of the conditions of the hypothesis, “Out” those violating any of them. A randomization test was run 10000 times. In each step, the response information was destroyed by randomly shuffling it while the gene expression data were left untouched. For each randomization, the hypothesis was evaluated and an odds ratio computed.

Out of 10000 odds ratios, 140 yielded an odds ratio of at least that of the original data (7.4). This relates to a significance of p=0.014 which means that the hypothesis is significant. The Positive Predictive Value is 50% for predicting TO status and 88% for predicting CR/PR and 100% for predicting N0 status. The Negative Predictive Values is 89% for predicting TO status. This algorithm has a specificity of 91% and a sensitivity of 44% for prediction of TO status.

However, one has to take into account, that the benefit of the combined chemo- and antibody therapy (Epirubicine Cyclophosphamide-Paclitaxel Herceptin, “EC-TH”) is only in part due to trastuzumab treatment. Indeed also in three major adjuvant trials (SABP B31, NCCTG N9831, HERA) the addition of trastuzumab to a very similar chemotherapy regimen (Doxorubicine Cyclophosphamide-Paclitaxel Herceptin, “AC-TH”) resulted in 50% less events with regard to Disease-free survival. Therefore, a predictor of solely Herceptin response should have a sensitivity of 50% at best, which is in concordance with the above described performance of the Herceptin response predictor.

For illustration of the results we have done 2D hierarchical clustering based on candidate genes of the 17q12, 8q24 and 11q12 ARCHEONs. As an example the clustering by using TRIB1 (8q24), c-Myc (8q24), MGC9753 (17q12) and ER we could discriminate four different groups of tumors, with the responding tumors being present in only two subgroups.

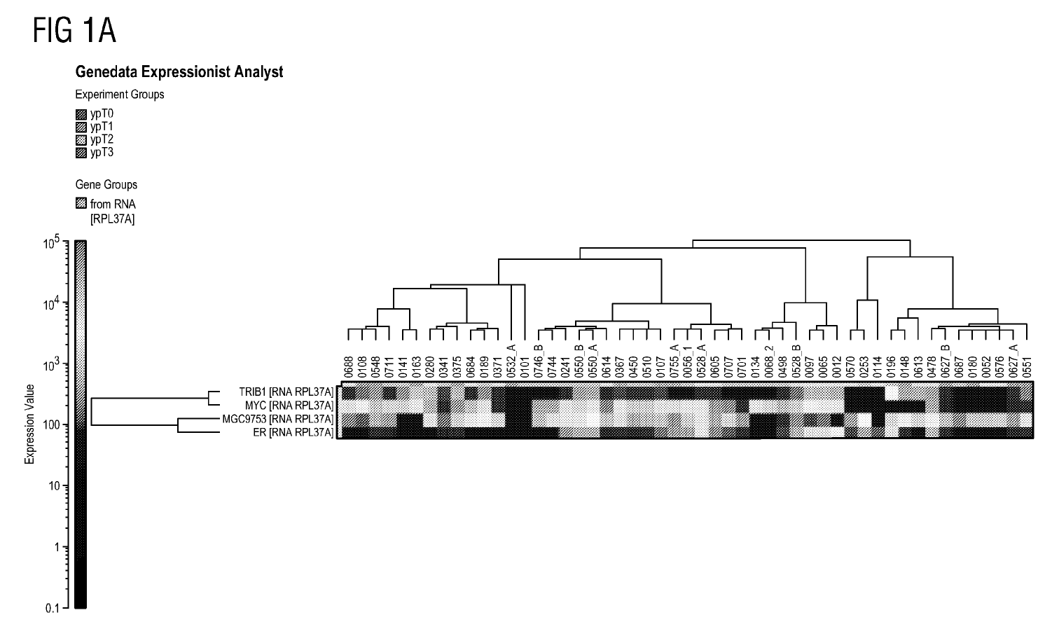

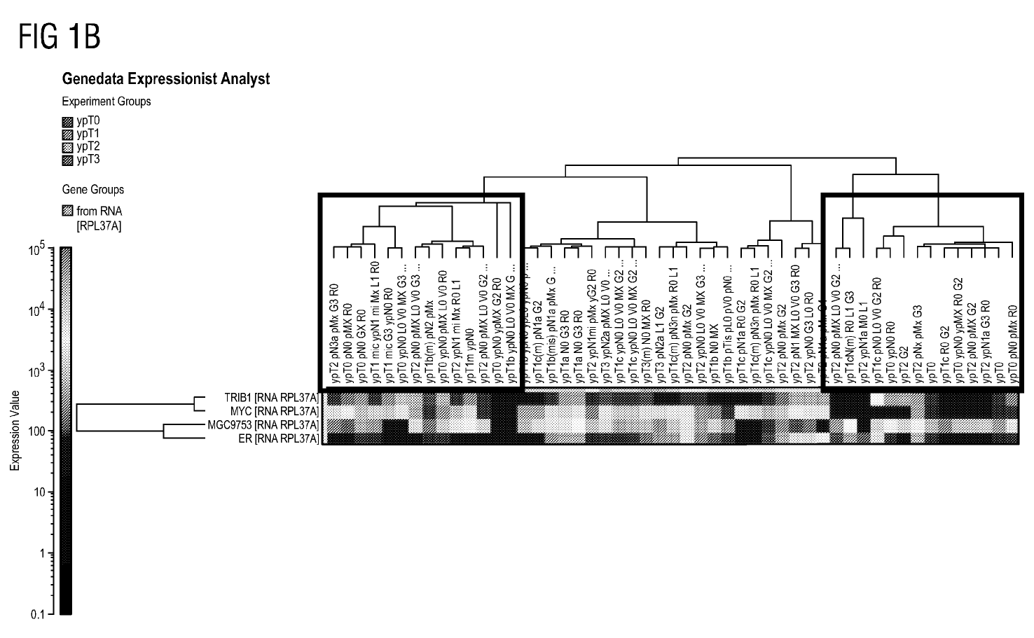

### FIG. 1a

### 2D Hierarchical Clustering Based on 3 ARCHEON Genes and ER

FIG. 1a: Analysis of candidate genes by 2D Hierarchical clustering based on relative expression of candidate genes as determined by RT-qPCR of formalin fixed paraffin embedded tissues from pretreatment core needle biopsies of primary tumors. Absolute expression levels are normalized by scaling of each sample to identical expression levels of the housekeeping gene RPL37A. Patients are depicted in rows and designated by the internal tumor sample number. Gene expression is shown in lines with the gene names and normalization mode depicted on the left of each line. The expression value is colour coded according to the scale depicted on the left with black for no expression, blue for low expression, green for medium expression and yellow/orange for high expression. (Tumors 0532A and 0101 were analyzed at lower detection limit; yet were not excluded for this analysis).

### FIG. 1b

### 2D Hierarchical Clustering Based on 3 ARCHEON Genes and ER

FIG. 1b: Analysis of candidate genes by 2D Hierarchical clustering based on relative expression of candidate genes as determined by RT-qPCR of formalin fixed paraffin embedded tissues from pretreatment core needle biopsies of primary tumors. Colour code is depicted on the upper left side to visualize clinical tumor response: Response of primary tumors after neoadjuvant treatment (=“y”) as assessed by pathohistological examination (=“p”) of tumor resectates is depicted (on top left and above columns) as “ypT0” in dark green (“TO”=no tumor cells detected; pCR), “ypT1” in light green (“T1”=tumor diameter of 1 cm; pPR), “ypT2” in orange (“T2”=tumor diameter of 2 cm; pSD) and “ypT3” in orange (“T1”=tumor diameter of 3 cm; pSD). Absolute expression levels are normalized by scaling of each sample to identical expression levels of the housekeeping gene RPL37A. Patients are depicted in rows and designated by the pathohistological data available at timepoint of analysis. Gene expression is shown in lines with the gene names and normalization mode depicted on the left of each line. The expression value is colour coded according to the scale depicted on the left with black for no expression, blue for low expression, green for medium expression and yellow/orange for high expression. Subgroups as defined by 2D hierarchical clustering with TRIB1, c-Myc, MGC9753 and ER, that did contain pCR (“ypT0” without in situ components) are marked by green boxes.

As can be seen in FIGS. 1a and 1b, most if not all tumors exhibit expression of MGC9753 (3^{rd} line), which we could show is only expressed in Her-2/neu (17q12 ARCHEON) positive tumors exhibiting a chromosomal alteration at 17q12. This is in line with the stratification criteria of this patient cohort in this trial: All patients have been centrally tested by IHC for Her-2/neu overexpression (DAKO HercepTest 3+ score). In case the IHC test detected a moderate overexpression (DAKO HercepTest 3+ score) the tumors had to be positively retested to be Her-2/neu positive by FISH analysis in order to include the patient into the neoadjuvant trial (EC-TH) as described above. In addition, approximately 60% of the tumors exhibit overexpression of c-Myc (2^{nd} line; 8q24 ARCHEON) as detected by RNA Analysis in FFPE core needle biopsies. However, higher expression of c-Myc (8q24 ARCHEON) did not correlate with good response to tratuzumab containing neoadjuvant treatment as suggested by researchers from the NSABP Operations and Biostatistical Center (see introduction). As multiple of the c-Myc positive tumors did not express TRIB1, which is also present on the 8q24 ARCHEON and cooverexpressed upon genomic alteration of this region, we conclude that c-Myc is expressed in a substantial number of Her-2neu positive breast tumors independently of genomica alteration. Therefore, the conclusion, that the pro-apoptotic function of dysregulated cMYC needs to be counterbalanced by an anti-apoptotic signal provided by Her-2/neu in order for such cells to develop into cancer and/or to circumvent therapeutically induced cell death seems not to be true. Instead, tumors exhibiting high c-Myc and TRIB1 expression (8q24 ARCHEON positive tumors) and lacking a prominent MGC9753 expression, which should give rise to a prominent tumor response, were resistant to the neoaduvant EC-TH regimen (see patient patients 0528B, 0097, 0066 and 0012). Interestingly, these tumors do express ER and therefore do have an independent mechanism for cell survival and proliferation based on hormonal activity. It is one assumption of these findings, that there is a need to address the ER/PR positive, Her-2/neu positive tumors also with anti-hormonal strategies (e.g. treatment with Tamoxifen, Raloxifen Faslodex or aromatase inhibitors such as exemestane, anastrozole, letrozole). It would in particular be interestingly to combine such chemo/antibody therapies with subsequent anti-hormonal treatment (most preferably exemestane) in the neoadjuvant setting to increase the tumor response. However, we have found that the expression of TRIB1, a gene being present on the 8q24 ARCHEON, has a pivotal role for sensitivity of tumors against EC-TH treatment. TRIB1 is a phosphoprotein being regulated by MAPK pathway downstream of the receptor tyrosine kinases.

As we have found, the ER negativity is a critical feature response to chemotherapy in conjunction with trastuzumab. Moreover we have found that the expression of Mikrotubule associated and regulating genes (TUBB, TUBB4, MAPT, STMN1, MAP4) is also important within the therapeutic setting of the TECHNO trial (“EC-TH”), which included a taxane.

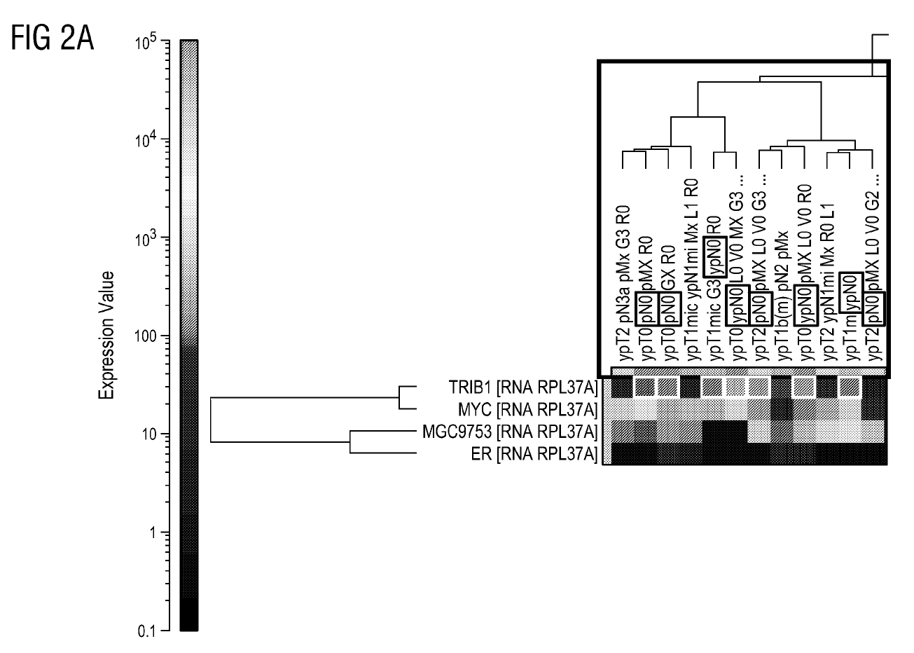

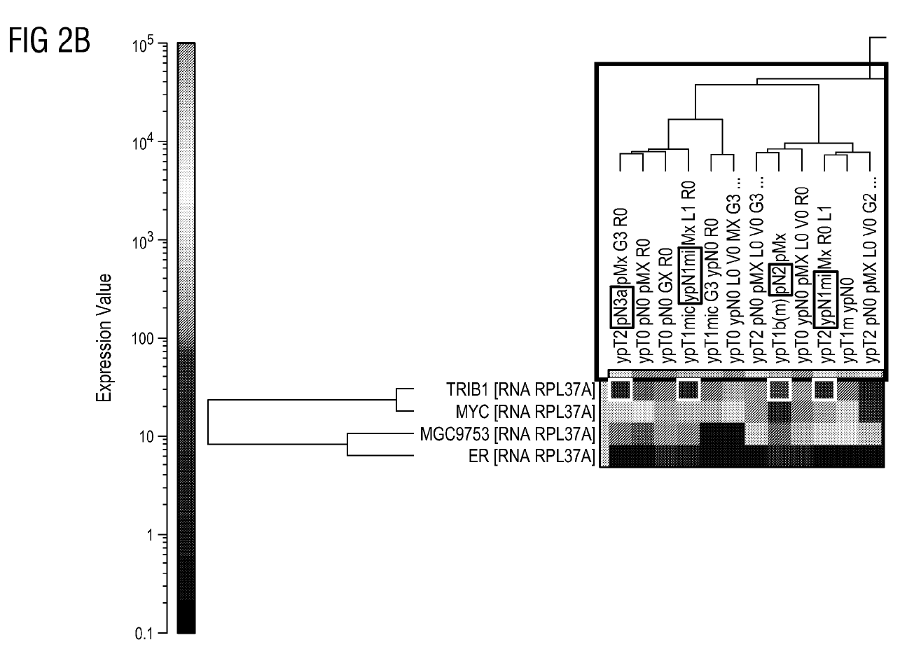

FIG. 2a

### Magnification of 2D Hierarchical Clustering Based on 3 ARCHEON Genes and ER

FIG. 2a: Magnification of analysis of candidate genes by 2D Hierarchical clustering based on relative expression of candidate genes as determined by RT-qPCR of formalin fixed paraffin embedded tissues from pretreatment core needle biopsies of primary tumors. Colour code is depicted on the upper left side to visualize clinical tumor response: Response of primary tumors after neoadjuvant treatment (=“y”) as assessed by pathohistological examination (=“p”) of tumor resectates is depicted (on top left and above columns) as “ypT0” in dark green (“TO”=no tumor cells detected; pCR), “ypT1” in light green (“T1”=tumor diameter of 1 cm; pPR), “ypT2” in orange (“T2”=tumor diameter of 2 cm; pSD) and “ypT3” in orange (“T1”=tumor diameter of 3 cm; pSD). Absolute expression levels are normalized by scaling of each sample to identical expression levels of the housekeeping gene RPL37A. Patients are depicted in rows and designated by the pathohistological data available at timepoint of analysis. Gene expression is shown in lines with the gene names and normalization mode depicted on the left of each line. The expression value is colour coded according to the scale depicted on the left with black for no expression, blue for low expression, green for medium expression and yellow/orange for high expression. Node negative Tumore are marked by green boxes. As can be seen there is a trend in the Trastuzumab Responder group with respect to the ARCHEON genes. In particular the responding tumors are: ER negative, 17q12 positive, myc positive, TRIB positive.

FIG. 2b: Magnification of analysis of candidate genes by 2D Hierarchical clustering based on relative expression of candidate genes as determined by RT-qPCR of formalin fixed paraffin embedded tissues from pretreatment core needle biopsies of primary tumors. Colour code is depicted on the upper left side to visualize clinical tumor response: Response of primary tumors after neoadjuvant treatment (=“y”) as assessed by pathohistological examination (=“p”) of tumor resectates is depicted (on top left and above columns) as “ypT0” in dark green (“TO”=no tumor cells detected; pCR), “ypT1” in light green (“T1”=tumor diameter of 1 cm; pPR), “ypT2” in orange (“T2”=tumor diameter of 2 cm; pSD) and “ypT3” in orange (“T1”=tumor diameter of 3 cm; pSD). Absolute expression levels are normalized by scaling of each sample to identical expression levels of the housekeeping gene RPL37A. Patients are depicted in rows and designated by the pathohistological data available at timepoint of analysis. Gene expression is shown in lines with the gene names and normalization mode depicted on the left of each line. The expression value is colour coded according to the scale depicted on the left with black for no expression, blue for low expression, green for medium expression and yellow/orange for high expression. Node positive Tumore are marked by red boxes. As can be seen there is a trend in the Trastuzumab Non-Responder tumors with respect to the ARCHEON genes. In particular the responding tumors are: ER negative, 17q12 positive, myc positive, TRIB negative.

This is in sharp contrast to the suggestions of the NSABP suggested that the pro-apoptotic function of dysregulated cMYC needs to be counterbalanced by an anti-apoptotic signal by another activated oncogene in order for such cells to develop into cancer. They claimed, that amplified HER-2/NEU may provide such anti-apoptotic signaling that is reduced by treatment with trastuzumab, resulting in triggering of apoptosis. All this analysis was done by detecting DNA amplifications of cMYC and HER-2/NEU by FISH technologies. In contrast, we have done RNA measurements of cMYC and HER-2/NEU. We have found, that overexpression of cMYC itself does not explain the good response to trastuzumab containing regimen, but is also apparent in non-responding tumors. However, we have found that low RNA expression of HER-2/NEU and its neighbouring genes (e.g. PPARBP and MGC9753) is to some extent informative for good response to treatment even though all tumors of the study were characterized to be IHC 3+ and or FISH positive. Therefore our technique provides additional information in a “homogenous” HER-2/NEU positive patient cohort. Still additional markers have to be evaluated for response prediction.

We have found high expression of genes neighbouring c-Myc in combination with genes located on 17q12, ER and Microtubule function associated genes are informative for the prediction of trastuzumab containing therapy regimens.

### REFERENCES

### Patents Cited

U.S. Pat. No. 6,379,895
WO 97/27317

### Publications Cited

(1) Gusterson et al., Journal of Clinical Oncology 10, 1049-1056, 1992
(2) Achuthan et al., Cancer Genet Cytogenet. 130:166-72, 2001

1. A method for the prediction, diagnosis or prognosis of malignant neoplasia by the detection of at least 2 markers characterized in that the markers are genes and fragments thereof or genomic nucleic acid sequences that are located on one chromosomal region which is altered in malignant neoplasia and the genes are selected from the group MLVI2 (5p14), NRASL3 (6p12), EGFR (7p12), c-myc (8q23), Cyclin D1 (11q13), IGF1R (15q25), HER-2/NEU (17q12), PCNA (20q12).

2. The method of claim 1 wherein the malignant neoplasia is breast cancer, ovarian cancer, gastric cancer, colon cancer, esophageal cancer, mesenchymal cancer, bladder cancer or non-small cell lung cancer.

3. A method for prediction diagnosis or prognosis of malignant neoplasia by the detection of at least 2 markers characterized in that the markers are selected from the group autoantibody against TG, autoantibody against TPO, serum HER-2/NEU, CRP, TG, T3, T4.

4. The method of claim 3 wherein the malignant neoplasia is breast cancer, ovarian cancer, gastric cancer, colon cancer, esophageal cancer, mesenchymal cancer, bladder cancer or non-small cell lung cancer.

5. The method of claim 3 wherein the malignant neoplasia is breast cancer treated with targeted therapies against the EGFR family and the VEGF/VEGFR system

6. The method of claim 5 wherein the targeted therapies are Trastuzumab (“Herceptin”), Lapatinib, Gefitinib (“Iressa”), cetuximab (“Erbitux”), Tarceva (“Erlotinib”), Vatalanib, EMD7200, Avastin, Nexavar (“Sorafenib”) and Sunitinib (“Sutent”).
